(19) United States
(12) Patent Application Publication
(10) Pub. No.: US 2005/0042643 A1
(43) Pub. Date: Feb. 24, 2005

(54) ELAVL-1

(75) Inventors: Tom Cotter, Cork (IE); Ian Hayes, Cork (IE); Liam Seery, Cork (IE); Finnbar Murphy, Cork (IE)

Correspondence Address: PALMER & DODGE, LLP;KATHLEEN M. WILLIAMS, 111 HUNTINGTON AVENUE, BOSTON, MA, 02199, US

(73) Assignee: EiRx Therapeutics, Limited

(21) Appl. No.: 10/856,647

(22) Filed: May 28, 2004

Related U.S. Application Data
(63) Continuation-in-part of application No. PCT/GB02/05393, filed on Nov. 29, 2002.

(30) Foreign Application Priority Data
Nov. 30, 2001 (GB) 0128754.9

(51) Int. Cl. C12Q 1/68; G01N 33/574; A61K 48/00

(52) U.S. Cl. 435/6; 435/7.23; 514/12; 514/44

(57) ABSTRACT

The invention provides a method for detecting apoptosis in a cell comprising detecting a decrease in any one of: i) an ELAVL-1 (embryonic lethal, abnormal vision, Drosophila-like 1) polypeptide having an amino acid sequences as set out in SEQ ID NO: 1; ii) a polypeptide having at least 80% homology with i); iii) a nucleic acid encoding a polypeptide having the sequence set out in i) or ii); iv) a nucleic acid which hybridises under stringent conditions to the sequence set out in iii); or v) the complement of iii) or iv). The invention accordingly provides a method of modulating apoptosis by modulating ELAVL-1 gene expression and a method for identifying genes associated with ELAVL-1 gene expression and thus identifying other genes associated with apoptosis. The invention also provides a novel nucleic acid sequence encoding the promoter region for ELAVL-1.

### FIELD OF THE INVENTION

The present invention relates to the use of the gene, ELAVL-1 (embryonic lethal, abnormal vision, Drosophila-like 1) in the detection and modulation of apoptosis in cells. In particular, it relates to a method for identifying genes associated with ELAVL-1 gene expression and thus their association with apoptosis. The present invention also relates to a novel nucleic acid sequence encoding the promoter region for ELAVL-1.

### BACKGROUND TO THE INVENTION

Programmed cell death or apoptosis is a genetically programmed process by which cells die under both physiological and a variety of pathological conditions (Kerr et al, Br. J. Cancer, 26, 239-257, 1972). It serves as the counter-balancing force to mitosis during adult life and is a major contributor to the sculpting of physiological structures during the many processes of development (Wyllie et al, Int Rev. Cytol, 68, 251-305, 1980). It is characterised by a number of well-defined biochemical hallmarks. These include DNA fragmentation, caused by the activation of an endogenous endonuclease enzyme (Wyllie, Nature, 284, 555-556,1980; Enari et al., Nature, 391, 43-50, 1998). The result is a DNA ladder pattern that can be readily visualised in agarose cells. Coupled with DNA fragmentation is cell shrinkage (Wesselbory et al., Cell Immunol. 148, 234-41, 1993) where water is actively extruded from the cell. The apoptotic cell then undergoes fragmentation into apoptotic bodies that are engulfed by neighbouring cells or cells of the reticuloendothelial system.

A second well-defined characteristic is the exposure of the phospholipid phosphatidylserine to the outside surface of the plasma membrane of the cell as it undergoes apoptosis (Fadok et al., J Immunol. 148, 2207-16, 1992). Normally this lipid is located on the inner side of the membrane lipid bilayer. The underlying mechanism responsible for this lipid flipping is poorly understood at present. Its expression serves as a signal for the recognition and phagocytosis of the apoptotic cell (Fadok et al., J Immunol. 148, 2207-16, 1992)

Under normal physiological conditions apoptosis is tightly regulated. However, there are a number of diseases where the process becomes deregulated, leading to a particular pathology. Examples of where apoptosis is retarded or inhibited include some types of tumour development, a number of inflammatory conditions such as acute respiratory distress syndrome (ARDS) and other related conditions (Matute-Bello et al, Am J Respir Crit Care Med. 56, 1969-77, 1997). Inappropriate or excessive apoptosis occurs under conditions of ischaemia (stroke, myocardial infarction, etc) Linnik et al., Blood. 80, 1750-7, 1992, Gorman et al., J Neurol Sci. 139, 45-52, 1996) a series of neurodegenerative conditions, myelosuppression (Mori et al, Blood. 92, 101-7, 1998) following chemotherapy or irradiation (Lotem et al., Blood. 80, 1750-7, 1992) and a significant number of other diseases where cell death is a key feature of the pathology.

A key event in the initiation and propagation of apoptosis is the generation of reactive oxygen species (ROS) (McGowan et al., Exp Cell Res, 238, 248-56, 1998). These species include hydrogen peroxide, superoxide and the hydroxyl radicals. They can be generated either at the level of the mitochondrion, where any disruption of the respiratory chain can lead to their production or via a number of enzyme reactions such as NADPH oxidase (Nauseef et al., Proc Assoc Am Physicians, 111, 373-82, 1999). This enzyme is particularly active in the neutrophil. Such molecules cause oxidative damage not only to cellular structures, but may also act to initiate the expression of apoptosis regulating genes. In mammalian cells the physiological role for such ROS molecules is far less well characterised than that of other related molecules such as nitric oxide (NO). In relation to the involvement of NO in apoptosis the published literature is unclear, with examples of NO both driving and inhibiting apoptosis (Brune et al., Cell Death Differ. 1999 10,969-975, 1999). There is an increasing volume of evidence for a ROS role in driving apoptosis, but, until recently, mechanisms of this has not been understood (Hildeman et al., Immunity. 10,735-44, 1999,Gorman et al., J Neurol Sci. 139, 45-52, 1996).

Studies over the past 5 years have demonstrated that a variety of cytokines, growth factors and agents that induce apoptosis can lead to the generation of ROS. These studies have suggested that ROS may act as second messengers in signal transduction pathways in the context of cytokine/growth factor stimulation of cells. Other more recent studies have indicated that they may also activate unique pathways. The specific targets of ROS generated intracellularly are largely unknown at present, but it is known that the addition of hydrogen peroxide or other ROS generators to cells in culture leads to the activation of the transcription factor Nf/kB (Schreck et al., EMBO J 10, 2247-2258, 1991). This in turn controls the expression of a series of genes involved in a variety of cellular functions. Other targets of ROS include the activation of the mitogen activated protein kinase (MAPK) which is known to be involved in the regulation of cell proliferation (Kamata et al., J. Biol. Chem, 271, 33018-33025).

A cell has the ability to produce ROS at a number of different sites. In relation to signal transduction events it is still unclear where the source of ROS is within the cell. There are a number of potential enzyme systems capable of ROS generation. Perhaps the best documented one, particularly in neutrophils and other phagocytic cells, is NADPH oxidase. Studies using inhibitors of this enzyme such as DPI suggest that this enzyme is also involved in the generation of ROS in non phagocytic cells (Griendling et al., Circulation, 74, 1141-1148, 1994). Mitochondria play a key role in apoptosis and are also a major site of ROS generation. The loss of mitochondrial membrane potential is coupled to the release of cytochrome C and this in turn has two effects. The first is the generation of ROS, since the respiratory chain is disrupted by the removal of cytochrome C. The second is the cleavage of cellular DNA through a series of cytochrome C mediated caspase activation steps, which is an end point of the apoptosis process.

It has been suggested that ROS are involved in p53 mediated apoptosis (Johnson et al., Proc. Natl. Acad. Sci. USA, 93, 11848-11852, 1997). Cells generated to over-express p53 undergo apoptosis, accompanied by ROS production and this can be blocked by anti-oxidants (Polyak et al., Nature, 389, 300-305, 1997). There are a number of other examples where ROS production is closely associated with the initiation and propagation of apoptosis. However, the mechanism of ROS activity in apoptosis has until recently been unclear.

A series of enzymes involved in maintaining the redox balance within a cell contribute to the ability of that cell to survive in the presence of elevated ROS levels. Such enzymes include catalase and superoxide dismutase which work to reduce the oxidative stress in cells. In addition to redox modulating enzymes several other proteins most notably Bcl-2 are thought to mediate their anti-apoptotic effects via an anti-oxidant process (Hockenberry et al., Cell. 1993 75 :241-51). The precise mechanism by which Bcl-2 mediates its effects are still not quite defined. Other proteins such as members of the heat-shock family have also been demonstrated to protect cells from undergoing apoptosis in a pro-oxidant environment (Creagh et al., Leukemia. 2000 (7):1161-73. The redox sensitive transcription factor NF-kB is also known to induce the expression of a series of genes (some known and others yet to be discovered) which modulate the cells ability to undergo apoptosis.

We have previously shown that production of intracellular ROS is causally or consequentially connected with the modulation of early transcription and/or translation, and/or post-translational modification in cells of genes which control the progression of the cell towards apoptosis (WO 01/46469). Unlike caspases and other genes known to be involved in apoptosis, which generally act at the execution stage of apoptosis and are only activated once the cell is committed to the apoptotic fate, the genes whose expression is modulated during or after ROS exposure are required for induction of apoptosis, before the cell has made a commitment to die. Accordingly, regulation of the expression of ROS-associated genes provides a mechanism by which the entry of the cell into the apoptotic process may be induced or prevented.

The number of factors which are know to induce survival in particular cell types is ever increasing (e.g. IL2, IL3, IL4, IL5, IL8, GM-CSF, insulin like growth factor 1, NGF, VEGF, PDGF, SCF, LIF, EGF etc.). Many of these survival factors appear to share a commonality in the survival pathway (Datta S R et al. Genes and Development 13: 2905-2927, 1999). For an extracellular stimuli, to confer survival on a cell, it must inhibit the endogenous apoptotic machinery. The model predicts that there is a series of temporal events that occur upon survival factor/receptor interaction. The first of these is tyrosine phosphorylation at the plasma membrane due either to intrinsic receptor tyrosine kinase activity (e.g. the insulin growth factor 1 receptor), or indirectly coupled to tyrosine kinases or alternatively directly coupled to several transmembrane G protein-coupled receptors.

Blood neutrophils have relatively short lives with greater than 80% of them apoptosing within the first 24 hours. Apoptotic neutrophils are phagocytosed by macrophages via thrombospondin and macrophage CD36/ vitronectin receptor (Savil 1992, Clinical Science 83, 649-55, Savil et al. 1993, Immunology Today 14,131-136) and thus prevent release of potentially lethal cocktail of enzymes in the host, should the neutrophil undergo necrosis. However, certain inflammatory environments favour the survival of neutrophils. In vitro, several cytokines including GM-CSF, IL-1, IL-2, IL-8 and IFNγ can delay neutrophil apoptosis (Brach et al, 1992, Blood 80, 2920-2924; Calotta et al 1992, Blood 80, 2012-2020, Lee et al 1993, J Leuk Biol 54, 283-388, Pericle et al 1994, Eur. J. Immunol24, 440-444, Get ref for IL8). Furthermore, inflammatory proteins (e.g. C5A) and bacterial products (e.g. LPS) have also been shown to inhibit apoptosis. These findings together with other results demonstrating that the presence of either actinomycin D or cycloheximide can promote apoptosis in PMN (Whyte et al, 1991, Clin Sci. 80:5p) suggests a role for active gene expression and translation in control of PMN apoptosis. Moreover, other investigators have shown that NFκB regulated genes seem to play a critical role in preventing apoptosis induced by TNFα, since inhibition of this transcription factor using the fungal metabolite Gliotoxin, induces rapid apoptosis (Ward et al. 1999, J. Biol. Chem. 274. 4309-4318). The same investigators also demonstrated that blocking NFκB with Gliotoxin removes the anti-apoptotic effect of LPS. Yoshida et al have identified an alternative mode of action of gliotoxin. These investigators demonstrated that gliotoxin inhibited NADPH oxidase and consequently prevented the onset of superoxide generation by human neutrophils in response to phorbol myristate. (Yoshida et al Biochem Biophys Res Commun 2000, 268(3) 716-23).

Granulocyte macrophage colony-stimulating factor (GM-CSF) is known to inhibit PMN apoptosis both in vitro and in vivo (Cox et al. 1992, Am. J. Respiratory Cell Mol Biol. 7, 507; Chintinis et al 1996, J. Leuk Biol 59:835). One consequence of GM-CSF treatment of PMN is a time and dose dependent tyrosine phosphorylation event within the cell (McCall et al., 1991, Blood 78(7) 1842-52). That tyrosine phosphorylation is implicated in the regulation of apoptosis has been demonstrated (Simon et al 1995, Int. Arch Allergy Immunol. 107, 338-339). These workers demonstrated that the effect of GM-CSF on granulocyte cell death could be attenuated by the tyrosine kinase inhibitor genestein, suggesting that increases in tyrosine phosphorylation are essential to inhibit cell death. To further analyse a role for tyrosine phosphorylation, the authors increased levels of tyrosine phosphorylation using the protein-phosphatase inhibitor phenylansine oxide (PAO). Similar to GM-CSF, treatment of the cells with PAO is followed by a large increase in tyrosine phosphorylation and matched inhibition of apoptosis. Inhibitors of tyrosine phosphorylation (Genestein and Herbimycin A) reversed the effects of PAO on tyrosine phosphorylation and neutrophil apoptosis.

Furthermore, Wei et al (J. Immunology 1996,157, 5155-5162) suggested specificity in the anti-apoptotic signalling pathway by showing that GM-CSF inhibition of programmed cell death did not appear to be related to known proteins associated with cell survival i.e. p53, cdc2, Rb, and Bcl-2. However GM-CSF did induce a rapid activation of Lyn, a src family tyrosine kinase, and Lyn antisense treatment of neutrophils reversed the survival promoting effect of GM-CSF. Other investigators have demonstrated that GM-CSF selectively induced tyrosine phosphorylation of Extracellular Signal-Related kinase (ERK), a member of microtubule associated protein kinase (MAPK) family (Yuo et al. 1997, BBRC 235, 42-46). Al-Shami et al. (Blood 1997, 89(3) 1035-1044) has shown that GM-CSF induces both a time and concentration- dependent increase in the level of tyrosine phosphorylation of the PI-3-kinase regulatory subunit p85, possibly via lyn kinase. In corroboration of these results, Klein et al. (J. Immunol. 2000, 164, 42864291), using pharmacological inhibitors of signal transduction, further demonstrated a role for PI 3-kinase and ERK. These investigators showed that GM-CSF caused a rapid phosphorylation of the protein Akt, a substrate for PI 3-kinase. Akt phosphorylation is in turn associated with phosphorylation of BAD, a pro-apoptotic member of the Bcl-2 family. The authors hypothesised that this phosphorylation resulted in disengagement of Bad with anti-apoptotic family members of Bcl-2 family, allowing them to prevent neutrophil apoptosis.

The link between GM-CSF and tyrosine phosphorylation and inhibition of programmed cell death has until recently been unknown. Previously, prolonged survival of PMN caused by inhibition of apoptosis is observed in bcl-2 transgenic mice (Lagasse and Weissman, 1994, J. Exp. Med. 179 1047). This result is surprising since normal peripheral blood neutrophils are negative for bcl-2 (Wei et al. J. Immunology 1996,157, 5155-5162), however it does show that targets for the Bcl-2 family of apoptosis associated proteins can control PMN apoptosis. Weinnman et al. (1999, Blood, 93, 3106-3115) investigated the role of other members of the Bcl-2 family in regulating PMN apoptosis. The authors cultured PMN for 0, 2,6 or 22 h in the presence of TNFα (pro-apoptotic) or GM-CSF or are left untreated. Fresh, unstimulated PMN showed a high level of expression of Bcl-XL that gradually decreased as the culture proceeded, suggesting that loss of this protective protein may play a role in spontaneous apoptosis. The reduction of Bcl-XL in the presence of TNFα is much stronger when compared to control cells. GM-CSF did not alter the effect of Bcl-XL. Next the investigators examined expression of Bax-α, a proapoptotic member of the Bcl-2 family. Results showed that GM-CSF induced a down regulation of Bax-α when compared to control cells, suggesting that the down-regulation of this death promoting is involved in PMN survival mediated by GM-CSF. The authors concluded that GM-CSF seems to promote survival by modulating the Bax-α/Bcl-XL ratio via down regulation of Bax-α. Furthermore, the authors suggested that inhibition of apoptosis by GM-CSF might be due to a caspase 3 regulation since no further reduction of apoptosis is observed, above that already seen, when PMN are stimulated GM-CSF after inhibition of caspase-3 with its inhibitor Z-DEVD-FMK.

Other members of the Bcl-2 family have also been implicated in neutrophil apoptosis. Expression of myeloid cell leukaemia 1 (MCL1), another viability-promoting family member, has been shown to decrease during neutrophil apoptosis but increases in response to GM-CSF and LPS, suggesting a link with PMN survival, (Moulding D A, Quayle J A, Hart C A, Edwards S W, Blood 1998; 92(7): 2495-502). Neutrophils also express mRNA for A1, another Bcl-2 homologue with anti-apoptotic properties (Chuang P I, Yee E, Karsan A, Winn R K, Harlan J M, Biochem Biophys Res Commun 1998; 249(2): 361-5). The authors demonstrated that agonists that promote cell survival (e.g. LPS and G-CSF) up-regulated the message for this protein. Moreover, neutrophil apoptosis is enhanced in mice that lack A1-a, a subtype of the A1 gene, and LPS-induced inhibition of apoptosis is abolished. However in these mice TNFα induced apoptosis is unchanged, which suggest that Al is involved in regulating some but not all neutrophil apoptotic pathways (Hamasaki A, Sendo F, Nakayama K, Ishida N, Negishi I, Nakayama Ki, Hatakeyama S, J Exp Med 1998; 188(11):1985-92).

In our copending international patent application, WO 02/04657, we have shown that GM-CSF inhibits death through apoptosis by the regulation of ‘effector genes’ that control the process of apoptosis. A signal acts through a signal transduction cascade and is associated with significant changes, or patterns of changes, in gene expression in the cell. To date, however, the identities of such ‘effector genes’ and their role in the signalling pathways that lead to the biochemical events of cell death have been incompletely determined.

Our previous work, as described in WO 01/46469 and WO 02/04657, therefore establishes two assays for the identification of genes involved in the regulation of apoptosis, by screening for genes whose expression is modulated by changes in intracellular ROS concentrations and/or the action of GM-CSF. Using these assays, we have verified changes in expression in a number of known genes whose role in apotosis has been previously established—such as various Bcl-2 related proteins and caspases. The assays therefore provide a method for validating the involvement of candidate genes in apoptosis.

The control of apoptosis represents a significant therapeutic target, since many diseases are due to defects in this process. Many physiological factors induce and prevent cell apoptosis. For example, cytokines or growth factors such as GM-CSF inhibit death through apoptosis. There is an acute need to identify the genes that regulate this process. In other words, if one identifies a gene that prevents apoptosis, then this gene/gene product or its function can be blocked by a drug and apoptosis allowed to occur. To-date many of the genes found have certain fundamental flaws e.g. they act late in the process, after the cell has committed to a death programme, or they are ubiquitous, that is they are not restricted to a particular cell type. The ideal target to control apoptosis act early in the process and are restricted to a particular cell type.

### SUMMARY OF THE INVENTION

The present invention identifies that the expression of the gene ELAVL-1 (embryonic lethal, abnormal vision, Drosophila-like 1) is correlated with an early stage in apoptosis. In particular, ELAVL-1 gene expression is decreased in neutrophil apoptosis and increased in neutrophil survival when apoptosis is inhibited by the presence of GM-CSF. Furthermore, when GM-CSF inhibition of apoptosis is blocked by gliotoxin, ELAVL-1 expression is down regulated. In addition, expression of recombinant ELAVL-1 in HeLa cells resulted in significant inhibition of proliferation/viability thus identifying ELAVL-1 as a modulator of cell growth/survival.

ELAVL-1, (embryonic lethal, abnormal vision, Drosophila-like 1; ELAV-like 1 or ELAVL1) also known as Hu-antigen R, HuR, Hua, HUR, MelG is identified in SwissProt, Accession number: Q15717; gi:13124204 and GenBank, Accession number: xm_{—}008947; gi1475941.The amino acid sequence is set out in SEQ ID NO: 1.

SEQ ID NO:1

| | | | | | | | |
|---|---|---|---|---|---|---|---|
| | 1 | 11 | 21 | 31 | 41 | 51 | |
| 1 | MSNGYEDHMA | EDCRGDIGRT | NLIVNYLPQN | MTQDELRSLF | SSIGEVESAK | LIRDKVAGHS | 60 |
| 61 | LGYGFVNYVT | AKDAERAINT | LNGLRLQSKT | IKVSYARPSS | EVIKDANLYI | SGLPRTMTQK | 120 |
| 121 | DVEDMFSRFG | RIINSRVLVD | QTTGLSRGVA | FIRFDKRSEA | EEAITSFNGH | KPPGSSEPIA | 180 |
| 181 | VKFAANPNQN | KNVALLSQLY | HSPARRFGGP | VHHQAQRFRF | SPMGVDHMSG | LSGVNVPGNA | 240 |
| 241 | SSGWCIFIYN | LGQDADEGIL | WQMFGPFGAV | TNVKVIRDFN | TNKCKGFGFV | TMTNYEEAAM | 300 |
| 301 | AIASLNGYRL | GDKILQVSFK | TNKSHK | | | | |

It has a ubiquitous tissue distribution and maps to chromosomal position 19p13.2. A mouse homologue has been identified which maps to chromosome 8, the mRNA sequence has been deposited in GenBank under accession number U65735 and nm_{—}010485.

The identification and role of this gene in apoptosis has been validated using model assays described in our copending applications WO 01/46469 and WO 02/04657as described herein.

These model discovery assays are configured to target the ‘early’ regulatory events occurring in apoptosis induced by ROS and, in particular, in the inhibition of apoptosis by GM-CSF. When apoptosis by GM-CSF is itself inhibited by a drug, such as gliotoxin, then changes, or patterns of changes can be targeted by clustering those changes that are common and both increase and/or decrease depending on the treatment. For example, a change that is a ‘decrease’ following induction of apoptosis is a candidate target gene, however, a change that is additionally an ‘increase’ following inhibition of apoptosis by GM-CSF has a higher probability of being a target gene because its regulation shows increased correlation with the process. Likewise, a change that is further a ‘decrease’ following inhibition of GM-CSF inhibitory effect has a yet higher probability of being a target gene because its regulation shows increased correlation with the process.

Genes regulated in these models following modulation of apoptosis include genes that 1) are ‘effector’ genes involved in the cells defence mechanisms aimed at preventing apoptosis (anti-apoptotic genes) and thus represent therapeutic targets, 2) make up aspects of the apoptosis and/or GM-CSF signal cascade and thus represent therapeutic targets, 3) initiate the process of apoptosis (pro-apoptotic genes) and thus represent therapeutic targets, and 4) are associated with the processes of apoptosis and defence that will aid in the understanding of key pathways, processes and mechanisms that may subsequently lead to the identification of therapeutic targets.

We have previously demonstrated that these cell-based apoptosis models, which are combined with a genomics approach, identify genes known to be involved in apoptosis and defence. In these models, ELAVL-1 expression correlates with that of known apoptosis genes, redox modulation and survival genes thus confirming its role in apoptosis or in modulating apoptosis. This role has not previously been identified. We therefore provide a method for detecting apoptosis in a cell comprising detecting a decrease in ELAVL-1 gene expression.

Accordingly in a first aspect of the invention, there is provided a method for detecting apoptosis in a cell comprising detecting a decrease in any one of:
i) an ELAVL-1 polypeptide having an amino acid sequence as set out in SEQ ID NO: 1;
ii) a polypeptide having at least 80% homology with i);
iii) a nucleic acid encoding a polypeptide having the sequence set out in i) or ii);
iv) a nucleic acid which hybridises under stringent conditions to the sequence set out in iii); or
v) the complement of iv).

Levels of gene expression may be determined in any appropriate manner. Detecting a decrease in gene expression may be achieved by measuring ELAVL-1 gene expression in treated versus non-treated cells. Preferably, gene expression may be measured by detecting nucleic acid encoding an ELAVL-1 polypeptide such as ELAVL-1 mRNA transcripts, or a fragment thereof. In one embodiment, the method of measuring mRNA transcripts may use an amplification technique as described herein. In another embodiment, ELAVL-1 expression may be measured by detecting the ELAVL-1 polypeptide gene product, or fragment thereof, using, for example, agents that bind ELAVL-1. Suitable agents include anti-ELAVL-1 antibodies.

In another aspect, there is provided a method of detecting GM-CSF-induced cell survival by detecting an increase in ELAVL-1 gene expression.

In another aspect, there is provided a method of modulating apoptosis in a cell comprising the step of increasing, decreasing or otherwise altering the functional activity of ELAVL-1 or the nucleic acid encoding it. In one embodiment, said modulation of apoptosis is inhibition. In another embodiment, said modulation of apoptosis confers survival in a cell.

In another aspect there is provided a method of modulating cell growth in a cell comprising the step of increasing, decreasing or otherwise altering the functional activity of ELAVL-1 or the nucleic acid encoding it.

In one embodiment, the modulation of cell growth is the inhibition of proliferation.

Suitably the cell may be a therapeutic target for the treatment of disease. For example, such a cell may be a cancer cell, a cell involved in an inflammatory disorder, a cell involved in an autoimmune disorder or in a neurodegenerative disorder.

In the context of the present invention the term ‘altered functional activity of ELAVL-1 or the nucleic acid encoding it’ includes within its scope increased, decreased or an otherwise altered activity of ELAVL-1 as compared with the native protein functioning in its normal environment, that is within a single cell under native conditions. In addition, it also includes within its scope an increased or decreased level of expression and/or altered intracellular distribution of the nucleic acid encoding ELAVL-1, and/or an altered the intracellular distribution of ELAVL-1 itself.

In one embodiment, the method of modulating apoptosis or cell growth involves decreasing ELAVL-1 gene expression. In a preferred aspect, the expression of ELAVL-1 is reduced by greater than 50%, 60%, 70%, 80%, 90% or more of its normal level in untreated cells.

Preferably, a decrease in ELAVL-1 gene expression may be effected by antisense expression. Other means of decreasing ELAVL-1 gene expression will be recognised by those skilled in the art and include introducing dominant negatives, peptides or small molecules including RNA molecules such as siRNA molecules which cause a decrease in gene expression through RNA interference. Suitable siRNA molecules are described in the Examples section herein.

In another embodiment, said method involves increasing ELAVL-1 gene expression and therefore increasing cell survival. In a preferred aspect, the expression of ELAVL-1 is increased by greater than 50%, 60%, 70%, 80%, 90%, 100%, 200%, 500% or more of its normal level in untreated cells.

Preferably, said method comprises providing an expression vector comprising a nucleic acid sequence encoding an ELAVL-1 polypeptide; introducing the expression vector into the cell and maintaining the cell under conditions permitting expression of the encoded polypeptide in the cell. As defined herein, a nucleic acid encoding ELAVL-1 or an EIAVL-1 polypeptide encompasses fragments thereof.

In a futher aspect, there is provided the use of ELAVL-1, or an agent that alters ELAVL-1 expression in a cell, in the modulation of apoptosis.

Suitably, apoptosis is assessed by expression of ELAVL-1 in a test system and measuring the impact on cell growth and viability.

In a further aspect, there is provided a method for identifying a molecule which interacts with ELAVL-1.

In the context of the present invention, molecules which ‘interact’ with ELAVL-1 include molecules which bind to ELAVL-1 either directly or indirectly. Methods for detecting those molecules include physical methods and molecular biology techniques as herein described. Suitable standard laboratory techniques will be familiar to those skilled in the art and include immunoprecipitation, immunoblotting and fluorescence techniques. One skilled in the art, will appreciate that this list is not intended to be exhaustive. Suitable molecular biology techniques include phage display and the yeast two-hybrid system described herein.

ELAVL-1 may itself be used to identify other candidate genes or proteins which are involved in apoptosis.

Accordingly, in another aspect, there is provided a method for identifying a gene product whose expression is modulated by the expression of ELAVL-1 comprising the steps of:
providing a vector encoding ELAVL-1
introducing said vector in a cell under conditions to promote expression of ELAVL-1
measuring global gene expression associated with ELAVL-1 expression

In one embodiment, said method further comprises the step of exposing said cell to conditions which promote apoptosis or cell survival prior to measuring global gene expression.

Advantageously, expression levels are assessed by measuring gene transcription. This is preferably carried out by measuring the rate and/or amount of specific mRNA production in the cell. A preferred embodiment of this aspect of the invention involves the use of arrayed oligonucleotide probes capable of hybridising to mRNA populations. Differences in hybridisation patterns of different mRNA populations may be used to identify genes which are differentially expressed in the two populations. Differential expression may include different expression patterns observed across a time course. The arrayed oligonucleotide probes are advantageously derived from cDNA or EST libraries, and represent genes which are expressed by the cells under investigation.

Levels of gene expression may be determined in any appropriate manner. Preferably, levels of gene expression may be determined by the measurement of protein production by mRNA translation to detect increases or decreases in the rate or amount of mRNA translation.

Preferably, global gene expression is measured by assaying gene transcription using a microarray. In another embodiment, global gene expression is measured by protein array.

An increasing number of reports suggest that translational regulation of gene expression is a feature of apoptosis. RNA stability is one of a number of mechanisms by which translational regulation can be achieved. As discussed above, members of the ELAVL family, including ELAVL-1 contain binding domains which bind to and stabilise mRNAs. Moreover, ELAVL-1 expression during neutrophil apoptosis correlates with a number of proteins known to effect RNA stability.

Accordingly, in accordance with another aspect of the invention, there is provided a method for detecting mRNAs whose stability or translational status is modified by ELAVL-1.

Suitably, said method comprises measuring and comparing the amount of polysomes in the presence of different amounts of ELAVL-1.

Another aspect of the invention is directed to the identification of agents capable of modulating ELAVL-1 gene expression or protein function In this regard, the invention provides assays for determining compounds that modulate the function and/or expression of ELAVL-1.

In one embodiment, the effect of such modulating agents on ELAVL-1 protein function may be detected by measuring RNA stability in the absence or presence of such agents.

Alternatively, the identification of agents capable of modulating ELAVL1 protein function can be detected by measuring the expression of a gene whose expression is regulated by ELAVL1.

Accordingly, in another embodiment, there is provided a method for identifying a compound that modulates ELAVL1 protein function comprising:
taking a cell expressing ELAVL1;
introducing a test compound; and
measuring global gene expression compared to expression in the absence of the test compound as an indication of modulation of ELAVL1 protein function.

In another aspect, there is provided a use of ELAVL-1 in an assay for identifying an agent which modulates apoptosis.

Accordingly, in one aspect there is provided a method for identifying a compound that modulates ELAVL1 protein function comprising:
taking a cell expressing ELAVL1;
transfecting said cell with a nucleic acid construct encoding an ELAVL1-regulated gene;
introducing a test compound; and
detecting expression of the ELAVL1-regulated gene compared to expression in the absence of the test compound as an indication of modulation of ELAVL1 protein function.

Suitably, a ‘cell expressing ELAVL1’ is a cell which has been transfected with a nucleic acid construct encoding ELAVL1 preferably by providing a vector encoding ELAVL1 and introducing said vector into a cell under conditions to promote expression of ELAVL1, as described above. Transfection may result in transient, stable or inducible expression of ELAVL1 using methods familiar to those skilled in the art or as described herein.

Suitably, the ELAVL1-regulated gene is selected from the group of genes identified in Example 14.

In yet another aspect, there is provided a system for screening for compounds that modulate ELAVL1 protein function, said system comprising a cell expressing ELAVL1 which is co-transfected with a nucleic acid construct encoding an ELAVL1-regulated gene or the promoter sequence of such a gene operably linked to a reporter gene.

Suitably said method can be used to identify compounds that enhance cell survival.

Cells useful in the methods of the invention may be from any source, for example from primary cultures, from established cell lines, in organ culture or in vivo. Cell lines useful in the invention include cells and cell lines of haematopoietic origin. Suitable cells include HeLa, U937 (monocyte), TF-1, HEK293 (T), primary cultures of neutrophils or cells having neutrophil characteristics, for example HL60 cells, murine FDCP-1, FDCPmix, 3T3, primary or human stem cells.

Where methods are therapeutic, cells may be disease-associated cells such as cancer, inflammatory, autoimmune or neurodegeration-associated cells.

The modulation of apoptosis can be for therapeutic purposes. Accordingly in another aspect of the invention there is provided a method of treatment of disease comprising administering a modulator of ELAVL1 gene expression or functional activity to an individual.

In another aspect, there is provided the use of a modulator of ELAVL1 expression or activity in the manufacture of a medicament for use in the treatment of disease.

Suitably, said modulator is an antisense molecule or an RNA molecule which mediates RNA interference and thus causes a decrease in ELAVL1 expression.

Suitable diseases include cancer, inflammation, autoimmune disease and neurodegenerative disorders.

A number of inflammatory diseases such as asthma, chronic obstructive pulmonary disease (COPD), Cystic Fibrosis (CF), Rheumatoid Arthritis (RA) and Inflammatory bowel disease (IBD) are characterised by a) elevated levels and expression of cytokines and growth factors that act predominantly on myeloid cells, b) prolonged survival of myeloid cells, and c) prolonged activation of myeloid cells.

Thus, increased numbers of activated myeloid cells such as neutrophils are associated with, and strongly implicated in, the pathology of a number of these chronic and acute inflammatory diseases (Williams T J and Jose P J: Novartis Found Symp 2001;234:136-41; discussion 141-8; Barnes P J: Chest 2000 February ;117(2 Suppl):10S-4S; Nadel J A: Chest 2000 February ;117(2 Suppl):10S-4S; Ward I et al: Trends Pharmacol Sci 1999 December ;20(12):503-9; Bradbury J and Lakatos L: Drug Discov Today 2001 May 1, ;6(9):441-442).

Accordingly, in one embodiment, there is provided the use of ELAVL1, or an agent that alters ELAVL1 expression in a cell in the treatment of inflammatory diseases through the modulation of myeloid cell apoptosis.

In the context of the present invention, the term “myeloid cell” refers to terminally differentiated, non-dividing cells of the myeloid lineage. These cells include neutrophils, eosinophils and monocytes/macrophages. In one embodiment of any aspect of the present invention, the myeloid cell is a neutrophil, eosinophil or monocyte/macrophage.

Inflammatory diseases include, but are not limited to, diseases such as sepsis, Acute Respiratory Distress Syndrome, Pre enclampsia, Myocardial ischemia, reperfusion injury, Psoriasis, Asthma, COPD, bronchiolitis, Cystic Fibrosis, Rheumatoid Arthritis, Inflammatory Bowel Disease, Crohns Disease and Ulcerative colitis.

In another aspect of the invention, there is provided an isolated nucleic acid molecule comprising a promoter, said nucleic acid sequence being selected from the group consisting of:
i) a nucleic acid molecule having the sequence set out in SEQ ID NO:2;
ii) a nucleic acid molecule having at least 60% homology with i);
iii) a nucleic acid molecule hybridising under stringent conditions to i) or ii); and
iv) the complement of the sequences set out in i) to iii).

The ELAVL-1 promoter region has been identified to comprise a number of sites which bind specific transcription factors or enhancers. Accordingly, in one embodiment there is provided a nucleic acid sequence as set out in i), ii) or iii) above which comprises one or more of the enhancer or transcription factor binding elements selected from the group consisting of MEF 2, AP2, GATA 1, Rad, TTS, BGRE, C/EBP, AP1 and Sp1 and including others known to the art but not specified herein. In another embodiment, said nucleic acid sequence comprises all of these enhancer or transcription factor binding elements.

Suitably activation of transcription from said nucleic acid sequence is regulated by GM-CSF.

In a particularly preferred embodiment, the promoter sequence comprises the sequence set out SEQ ID NO:2. In another embodiment, the promoter sequence comprises the sequence in the deposit made to ECACC, Accession No. 01112818 (Submitted 28 Nov. 2001).

Suitably the promoter sequence comprises the region of the genomic sequence from −1986 to +10 of the transcription start site for ELAVL-1.

In another aspect of the invention, there is provided a vector comprising a nucleic acid as defined above.

Preferably, said vector comprises a nucleic acid in accordance with the invention operably linked to a reporter gene.

In another embodiment the vector may further comprise other sequences such as sequences encoding selectable markers.

In a further aspect of the invention, there is provided a method of identifying a compound that modulates expression from the ELAVL-1 promoter comprising
transfecting a cell with a nucleic acid in accordance with the invention operably linked to a reporter gene;
introducing a compound of interest;
detecting ELAVL-1 gene expression by detecting the reporter gene product; and
comparing with ELAVL-1 gene expression in the absence of the compound of interest.

Suitably modulation of expression may be an increase (activation) or a decrease (inhibition) of expression from the ELAVL-1 promoter.

In another aspect of the invention, there is provided an isolated nucleic acid molecule selected from the group consisting of:
i) a nucleic acid molecule encoding an ELAVL-1 polypeptide having the sequence as set out in SEQ ID NO: 1 but having a single nucleotide polymorphism mapping to amino acid 268;
ii) a nucleic acid molecule having at least 80% homology with i);
iii) a nucleic acid molecule hybridising under stringent conditions to i) or ii); and
iv) the complement of the sequences set out in i) to iii).

### BRIEF DESCRIPTION OF THE DRAWINGS

FIG. 1 shows the dose responsiveness of the anti-apoptotic effect of GM-CSF. Optical densities are read at 570 nm using a plate reader. The results indicate a direct correlation between survival and concentrations of GM-CSF added to the culture medium.

FIG. 2 shows that the fungal metabolite gliotoxin blocks the GM-CSF in the inhibition of neutrophil apoptosis. The method is as described in Example 1. Optical densities are read at 570 nm using a plate reader. Gliotoxin effectively blocks the GM-CSF mediated inhibition of neutrophil apoptosis. The blocking effect is not seen when the inactive analogue of gliotoxin, methylgliotoxin is added with GM-CSF. No increased neutrophil apoptosis is seen with the addition of gliotoxin alone to isolated neutrophils, demonstrating that the effect is specific to, and limited to, a reversal of the protective effects of GM-CSF.

FIG. 3 shows a phosphoimage scan of a microarray.

FIG. 4 shows the analysis of a captured image film by Array Vision™ software.

FIG. 5 shows the results of combined code cluster analysis.

FIG. 6 shows cluster analysis of LifeGrid filters. Human purified peripheral blood neutrophils are either allowed to undergo spontaneous apoptosis (Apop), or else are treated with 5U/ml GM-CSF to inhibit apoptosis (GM-CSF). Samples are isolated for RNA extraction and microarray gene analysis, 2 h (Apop2 and GMCSF2), 3 h (Apop3), 4 h (Apop4 and GMCSF4), 5 h (Apop5) and 6 h (Apop6 and GMCSF6) post-isolation. In some experiments Gliotoxin (0.1 μg/ml; Glio) or its inactive analogue Methyl Gliotoxin (0.1 μg/ml; Methyl) are added in the presence of GM-CSF. Average fold change values (from two spots on the filters) for selected candidate apoptosis/survival-associated genes are compared to time zero controls (except GM4 which compares fold change of 4 h treatment of GM-CSF plus Gliotoxin with 4 h treatment of GM-CSF with Methyl Gliotoxin control), are analysed by GeneMaths using a Pearson correlation and Ward cluster algorithms. Increased expression (light) and decreased expression (dark) are represented and referenced by a color scale bar. ELAVL-1 gene is highlighted in bold.

FIG. 7 shows ELAV-like 1 mRNA is increased in GM-CSF-induced neutrophil survival, and this increased expression is blocked by Gliotoxin. Human purified peripheral blood neutrophils are treated as described in FIG. 6. The relative amounts of ELAVL-1 transcripts are shown.

FIG. 8 shows a dendrogram representation of cluster analysis for FIG. 6. Marker genes, with known function in apoptosis and survival are indicated. ELAVL-1 gene is highlighted in bold.

FIG. 9 shows cluster analysis of ELAVL-1 expression compared to two other known regulators of RNA stability. Human purified peripheral blood neutrophils are treated as described for FIG. 6. Average normalised sDens (from two spots on the filters) for all (>8,000) genes on the Incyte LifeGrid filters are analysed by GeneMaths using a Pearson correlation and Ward cluster algorithms. Expression levels are represented and referenced by a color scale bar. ELAVL-1 gene is indicated.

FIG. 10 shows ELAVL-1 expression correlates with Bcl-2 expression. Human purified peripheral blood neutrophils are treated as described for FIG. 6. Average fold change (from two spots on the filters) for all (>8,000) genes on the Incyte LifeGrid filters are compared to time zero controls (except GM4 which compares fold change of 4 h treatment of GM-CSF plus Gliotoxin with 4 h treatment of GM-CSF with Methyl Gliotoxin control), are analysed by GeneMaths using a Pearson correlation and Ward cluster algorithms. Increased expression (light) and decreased expression (dark) are represented and referenced by a color scale bar. ELAVL-1 gene is indicated.

FIG. 11 shows a time course of neutrophil differentiation, measured by NBT reduction assay. HL-60 cells are seeded at 5×10⁵/ml and incubated with 10 μM Retinoic acid for 24, 48 and 72 hours, or untreated (HL60). Cells are harvested and stimulated with PMA and incubated for 15 minutes in the presence of NBT. Approximately 5×10⁴ cells are transferred by cytospin onto slides and counter stained with Eosin. Slides are analysed blind and only whole cells containing formazan deposits were considered positive. Graph demonstrates differentiation towards the neutrophil lineage, as measured by percentage NBT positive cells.

FIG. 12 shows a time course of neutrophil apoptosis, following differentiation of HL60 cells. HL60 cells are seeded at 5×10⁶cells in T25 flasks and incubated with 10 μM Retinoic acid for 24, 48, 72, 96, 120 and 144 hrs. Mock treated HL-60 cells were used as a control. Cellular DNA was analysed for fragmentation into oligonucleosomal-size fragments and their multiples by agarose gel electrophoresis. Lanes 1,3,5,7,9,11 contain DNA from HL-60 cells that were differentiated with 10 μM Retinoic Acid over the indicated period whereas samples in lanes 2,4,6,8,10,12,14 are from control HL-60 cells treated for the same time period. Lane 15 is a 1 Kb Plus Ladder Molecular Marker.

FIG. 13 shows ELAVL-1 is differentially regulated during neutrophil diferentiation. Human purified peripheral blood neutrophils are treated as described in FIG. 11. cDNA is hybridised on Incte LifeGrid filters. Average fold change (from two spots on the filters) for ELAVL-1 gene on the Incyte LifeGrid filters are compared to time zero controls.

FIG. 14 shows ELAVL-1 gene expression is decreased by cisplatin-induced apoptosis in HeLa cells. HeLa cells are plated into 75 cm² flasks (6×10⁶ cells/flask) and allowed to adhere for four hours. After this period, cells are treated with Cisplatin (1 ug/ml) and incubated at 37° C. RNA samples are isolated and analysed by microarray, using Incyte LifeGrid filters at 0 h, 2 h and 4 h following the addition of cisplatin. Average fold change of the ELAVL-1 gene at 2 h and 4 h, compared with 0 h is indicated.

FIG. 15 shows the nucleotide sequence for the ELAVL-1 promoter. The underlined sequence represents the beginning of the mRNA transcript

FIG. 16 shows enhancer and transcription factor binding elements in the ELAVL-1 promoter.

FIG. 17 shows a graphical representation of the effect of known survival and pro-apoptotic genes on the proliferation/viability of HeLa cells, as determined by a plaque assay. HeLa cells are either untransfected (Mock), or transfected with control plasmids (Null=empty pcDNA vector, EGFP, the tumor suppressors p53 and p7³, and the survival genes SOD and glutathione peroxidase). Cells were quantified by crystal violet staining and measurement of Abs 570 nm are the mean±standard error of three wells.

FIG. 18 shows a graphical representation of the effect of ELAVL-1 on the proliferation/viability of HeLa cells, as determined by a plaque assay. HeLa cells are either untransfected (Mock), or transfected with control plasmids (Null=empty pcDNA vector, EGFP, and the tumor suppressor p53), or ELAV like 1 (ELAV). Cells were quantified by crystal violet staining and measurement of Abs 570 nm are the mean±standard error of three wells.

FIG. 19 shows expression of ELAVL-1 in HeLa cells confers resistance to cisplatin-induced cell death. Cell clonagenicity is measured in HeLa cells stably expressing ELAVL-1 full-length cDNA (A) or mitochondrial superoxide dismutase (SOD; B) compared to control cells transfected with vector containing EGFP, following treatment with a range of cisplatin concentrations (0.0 to 10 μg/ml). Cells are plated at the indicated cell concentration and after adhering 24 h are treated with various doses of cisplatin for a further 24 h. Cisplatin is then removed and cells cultured for a further 96 h when cell survival is determined by MTT. Results are mean of three separate wells.

FIG. 20 shows a visual representation of a cluster analysis of comparative transient transfection of HeLa cells with full-length cDNAs for ELAVL-1, p53 and Bcl-2, HB24, TTF and SOD. HeLa cells are transiently transfected with pcDNA3.1 containing full-length cDNA for ELAVL-1, or Bcl-2, HB24, TTF, SOD and p53 using the CalPhos Mammalian Transfection Kit (Clontech) according to the manufacturers instructions. RNA is isolated for microarray profiling ˜42 h following the addition of the transfection reagent Each data set represents the expression of >8,000 individual genes from Incyte LifeGrid array filters. For each experiment average fold change values are calculated relative to transient transfected ‘empty’ control expression vector. Average fold change values (from two spots on the array filters) are clustered with GeneMaths, using a Pearson correlation and Ward clustering algorithm. Cluster analysis is both between individual genes on the array and also between arrays. Therefore, similar overall expression patters across the entire array can be associated. The Fold Change colour scale is shown.

FIG. 21 shows a subset of the visual representation of a cluster analysis of comparative transient transfection of HeLa cells with full-length cDNAs for ELAVL-1, p53 and Bcl-2, HB24, TTF and SOD, represented in FIG. 20 and demonstrates that transient expression of recombinant ELAVL-1 is associated with significant changes in gene expression.

FIG. 22 shows expression of ELAVL-1 in HeLa cells confers resistance to hydrogen-peroxide induced cell death. Cell Clonagenicity is measured in HeLa cells stably expressing ELAVL-1 full length cDNA(A) or mitochondrial superoxide dismutase. (SOD; B) compared to control cells transfected with vector containing EGFP, following treatment with a range of cisplatin concentrations (0-1320 μM). Cells are plated at the indicated cell concentration and after adhering 24 h are treated with the various doses of hydrogen peroxide for a further 24 h. Cisplatin is then removed and cells cultured for a further 96 h when cell survival is determined by MTT.

FIG. 23 shows ELAVL1 siRNA reduced the expression of ELAVL1 mRNA in HeLa cells. A. Relative amounts of ELAVL1 in transfected cells. B. Normalised ELAVL1 levels in ELAVL1 siRNA transfected HeLa cells. C. Relative amount of ELAVL1 mRNA expressed as a percentage of ELAVL1 levels in Lamin A/C siRNA transfected HeLa cells.

FIG. 24 shows TF1 cells transfected with ELAVL1 siRNA. A. Relative amounts of ELAVL1 in transfected cells. B. Normalised ELAVL1 levels in ELAVL1 siRNA transfected Hela cells. C. Relative amount of ELAVL1 mRNA expressed as a percentage of ELAVL1 levels in Lamin A/C siRNA transfected Hela cells.

FIG. 25 shows ELAVL1 siRNA reduced the viability of TF1 cells as measured by A. cell counts, B. FSC/SSC profile and C. Percentage Sub-G1.

FIG. 26 shows ELAVL1 siRNA reduced the viability of HeLa cells as measured by A MTT results, B Forward/Side Scatter results, and C Sub G1 analysis.

### DETAILED DESCRIPTION OF THE INVENTION

Unless defined otherwise, all technical and scientific terms used herein have the same meaning as commonly understood by one of ordinary skill in the art (e.g., in cell culture, molecular genetics, nucleic acid chemistry, hybridisation techniques and biochemistry). Standard techniques are used for molecular, genetic and biochemical methods. See, generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4^{th} Ed, John Wiley & Sons, Inc.; as well as Guthrie et al., Guide to Yeast Genetics and Molecular Biology, Methods in Enzymology, Vol. 194, Academic Press, Inc., (1991), PCR Protocols: A Guide to Methods and Applications (Inns, et al. 1990. Academic Press, San Diego, Calif.), McPherson et al., PCR Volume 1, Oxford University Press, (1991), Culture of Animal Cells: A Manual of Basic Technique, 2nd Ed. (R. I. Freshney. 1987. Liss, Inc. New York, N.Y.), and Gene Transfer and Expression Protocols, pp. 109-128, ed. E. J. Murray, The Humana Press Inc., Clifton, N.J.). These documents are incorporated herein by reference.

Definitions

“Apoptosis” or programmed cell death is a controlled intracellular process characterised by the condensation and subsequent fragmentation of the cell nucleus during which the plasma membrane remains intact. It is an active, highly regulated process distinguished by cell shrinkage and packaging of the cell contents into apoptotic bodies that are subsequently engulfed by macrophages, thus avoiding activation of the inflammatory response (for review see Wyllie, Br. Med. Bull. 53:451-465, 1997). Apoptotic death is distinct from other cell processes including necrotic cell death and replicative senescence. Replicative senescence, in contrast, is the state cells enter, prior to cell death, following a finite number of mitotic cycles (Hayflick and Moorhead, Exp.Cell Res. 25: 585-621, 1961). This phenomenon has been observed in almost all division-competent cells susceptible to cell culture (Pereira-Smith and Bertram, Generations 24:43, 2000). There are several extant theories to account for the mechanism of replicative senescence, although recent data favours a chromosomal telomerase shortening model (Campisi, In Vivo, 14:183-188, 2000; Wright and Shay, Curr Opin Genet Dev, 11:98-103, 2000). Replicative senescence is not cell death (Bree et al., BioGerontology 3: 195-206, 2002). Indeed, senescent cells are resistant to apoptosis and may remain metabolically active and live for up to three years in vitro (Pereira-Smith and Bertram, 2000, supra.).

By “modulating apoptosis” is meant that for a given cell, under certain environmental conditions, its normal tendency to undergo apoptosis is changed compared to an untreated cell. For example, blood neutrophils have a defined apoptotic tendency—within a population of cells, greater than 80% will apoptose within the first 24 hours. Modulating the apoptosis of blood neutrophils means changing this normal apoptotic tendency such that apoptosis is increased or decreased relative to the normal rate. Similarly, blood neutrophils in the presence of GM-CSF have a decreased tendency to apoptose. Thus, modulating apoptosis of blood neutrophils in the presence of GM-CSF means increasing or decreasing apoptosis relative to their normal decreased tendency under these conditions. A decreased tendency to apoptose may also be a measurable increase in cell survival and may be the result of an inhibition of apoptosis by inhibiting one or more components of the apoptotic pathway.

The term “expression” refers to the transcription of a genes DNA template to produce the corresponding mRNA and translation of this mRNA to produce the corresponding gene product (i.e., a peptide, polypeptide, or protein). The term “activates gene expression” refers to inducing or increasing the transcription of a gene in response to a treatment where such induction or increase is compared to the amount of gene expression in the absence of said treatment. Similarly, the terms “decreases gene expression” or “down-regulates gene expression” refers to inhibiting or blocking the transcription of a gene in response to a treatment and where such decrease or down-regulation is compared to the amount of gene expression in the absence of said treatment.

“Antibodies” can be whole antibodies, or antigen-binding fragments thereof. For example, the invention includes fragments such as Fv and Fab, as well as Fab′ and F(ab′)₂, and antibody variants such as scFv, single domain antibodies, Dab antibodies and other antigen-binding antibody-based molecules.

The “functional activity” of a protein in the context of the present invention describes the function the protein performs in its native environment. Altering the functional activity of a protein includes within its scope increasing, decreasing or otherwise altering the native activity of the protein itself. In addition, it also includes within its scope increasing or decreasing the level of expression and/or altering the intracellular distribution of the nucleic acid encoding the protein, and/or altering the intracellular distribution of the protein itself.

The terms “variant” or “derivative” in relation to ELAVL-1 polypeptide includes any substitution of, variation of, modification of, replacement of, deletion of or addition of one (or more) amino acids from or to the polypeptide sequence of ELAVL-1. Preferably, nucleic acids encoding ELAVL-1 are understood to comprise variants or derivatives thereof.

The term “nucleic acid”, as used herein, refers to single stranded or double stranded DNA and RNA molecules including natural nucleic acids found in nature and/or modified, artificial nucleic acids having modified backbones or bases, as are known in the art.

An “isolated” nucleic acid, as referred to herein, refers to material removed from its original environment (for example, the natural environment in which it occurs in nature), and thus is altered by the hand of man from its natural state. For example, an isolated polynucleotide could be part of a vector or a composition of matter, or could be contained within a cell, and still be “isolated” because that vector, composition of matter, or particular cell is not the original environment of the polynucleotide. Preferably, the term “isolated” does not refer to genomic or cDNA libraries, whole cell total or mRNA preparations, genomic DNA preparations (including those separated by electrophoresis and transferred onto blots), sheared whole cell genomic DNA preparations or other compositions where the art demonstrates no distinguishing features of the nucleic acids of the present invention.

“Vector” refers to any agent such as a plasmid, cosmid, virus, autonomously replicating sequence, phage, or linear single-stranded, circular single-stranded, linear double-stranded, or circular double-stranded DNA or RNA nucleotide sequence that carries exogenous DNA into a host cell or organism. The recombinant vector may be derived from any source and is capable of genomic integration or autonomous replication.

The term “promoter” or “promoter region” refers to a nucleic acid sequence, usually found upstream (5′) to a coding sequence, that is capable of directing transcription of a nucleic acid sequence into mRNA. The promoter or promoter region typically provide a recognition site for RNA polymerase and the other factors necessary for proper initiation of transcription. As contemplated herein, a promoter or promoter region includes variations of promoters derived by inserting or deleting regulatory regions, subjecting the promoter to random or site-directed mutagenesis, etc. The activity or strength of a promoter may be measured in terms of the amounts of RNA it produces, or the amount of protein accumulation in a cell or tissue, relative to a promoter whose transcriptional activity has been previously assessed.

A “nucleic acid encoding the promoter sequence of ELAVL-1” means a nucleic acid which is capable of directing transcription of ELAVL-1 gene expression. The term moreover includes those polynucleotides capable of hybridising, under stringent hybridisation conditions, to the naturally occurring nucleic acids identified above, or the complement thereof.

“Stringent hybridisation conditions” refers to an overnight incubation at 42° C. in a solution comprising 50% formamide, 5×SSC (750 mM NaCl, 75 mM trisodium citrate), 50 mM sodium phosphate (pH 7.6), 5× Denhardt's solution, 10% dextran sulphate, and 20 pg/ml denatured, sheared salmon sperm DNA, followed by washing the filters in 0.1×SSC at about 65° C.

The phrase “operably linked” refers to the functional spatial arrangement of two or more nucleic acid regions or nucleic acid sequences. For example, a promoter region may be positioned relative to a nucleic acid sequence such that transcription of a nucleic acid sequence is directed by the promoter region. Thus, a promoter region is “operably linked” to the nucleic acid sequence.

“Transcription” refers to the process of producing an RNA copy from a DNA template. As discussed above, a promoter or promoter region for a gene typically provides a recognition site for RNA polymerase and for the other factors, such as transcription factors or enhancers, which are necessary for proper initiation of transcription. The ELAVL-1 promoter region has been identified to comprise a number of sites which bind specific transcription factors or enhancers.

A “reporter gene” is a gene which is incorporated into an expression vector and placed under the same controls as a gene of interest to express an easily measurable phenotype.

The term “myeloid cell” encompasses terminally differentiated, non-dividing (i.e. non-proliferative) cells derived from the myeloid cell lineage and includes neutrophils or polymorphonuclear neutrophils (PMNs), eosinophils and mononuclear phagocytes. The latter cells are known as monocytes when in the blood and macrophages when they have migrated into the tissues. Terminal differentiation is the normal endpoint in cellular differentiation and is usually not reversible.

“Inflammatory disorders” or “inflammatory diseases” are disorders characterised by chronic or acute inflammation. This, in turn, is characterised by elevated levels of cytokines and/or survival factors for myeloid cells. These disorders are characterised by the prolongued survival of myeloid cells including neutrophils, eosinophils and monocytes/macrophages which can be present as a mixture of one or more of these cell types. Accordingly, reference to treatment of inflammatory disorders or diseases includes treatment of the individual cell types or treatment of a mixture of different cell types. The resultant increased numbers of these inflammatory cells is associated with the disease pathology. In chronic inflammation a persistent inflammatory response causes damaging effects such as tissue damage. Chronic Inflammatory Diseases include cystic fibrosis, acute respiratory distress syndrome, chronic obstructive pulmonary disease, inflammatory bowel disease and rheumatoid arthritis. Other inflammatory diseases are known to those skilled in the art and include sepsis, Pre enclampsia, Myocardial ischemia, reperfusion injury, Psoriasis, Asthma, bronchiolitis, Crohns Disease and Ulcerative colitis.

ELAVL-1 (Embryonic Lethal, Abnormal Vision Drosophila-Like 1)

Members of the ELAVL protein family contain 3 RNA-binding domains and bind to Cis-acting AU-rich elements (AREs). AREs destabilize mRNAs and play an important role in the control of gene expression. By RT-PCR of HeLa cell mRNA using primers based on a conserved region of HuD (ELAVL4), Ma et al. (J. Biol. Chem. 271: 8144-8151, 1996) isolated a partial cDNA encoding a novel protein that they designated HuR. They recovered additional cDNAs corresponding to the entire coding region and determined that the predicted HuR protein contains 326 amino acids. Like other ELAVL proteins, HuR has a short N-terminal region followed by 2 RNA-binding motifs, a basic linker domain, and a third RNA-binding motif Recombinant HuR protein bound to the ARE in both cytokine and oncogene mRNAs with high specificity and affinity. RT-PCR analysis indicated that HuR is expressed ubiquitously, in contrast to the neural-specific expression of ELAVL2, ELAVL3, and ELAVL4.

Four mammalian proteins have been identified that bind regions of HuR known to be essential for its ability to shuttle between the nucleus and the cytoplasm and to stabilize mRNA: SETalpha, SETbeta, pp32, and acidic protein rich in leucine (APRIL) (Brennan C M et al. J Cell Biol. 2000 Oct. 2;151(1):1-14)

Cytokine stimulation of human DLD-1 cells resulted in a marked expression of nitric-oxide synthase (NOS) II mRNA and protein accompanied by only a moderate increase in transcriptional activity. The embryonic lethal abnormal vision (ELAV)-like protein HuR was found to bind with high affinity to the adenylate/uridylate-rich elements of the NOS II 3′-UTR. Inhibition of HuR with antisense constructs reduced the cytokine-induced NOS II mRNA, whereas overexpression of HuR potentiated the cytokine-induced NOS II expression. (Rodriguez-Pascual F et al. J Biol Chem. 2000 Aug. 25; 275(34):26040-9).

Neuronal specific ELAV-like proteins have been implicated in regulating neuronal differentiation. Chicken HuD is expressed in maturing neurons and misexpression in cultured neural crest cells results in a dramatic increase in the proportion of cells exhibiting neuronal morphology, molecular markers for neurons, and neurotrophin dependence. (Wakamatsu Y and Weston J A. Development. 1997 September ;124(17):3449-60).

In Xenopus, three nervous system-specific elav/Hu related genes, elrB, elrC and elrD, have been identified. The temporally regulated expression patterns of elrB, elrC and elrD suggest their involvement at different steps of neural differentiation. Misexpression of elrB induces severe defects in neural tube development (Perron M et al. Int J Dev Biol. 1999 July ;43(4):295-303).

ELAVL-1 binds to the Early Response Gene class of mRNA, shuttling these to the cytoplasm in response to regulatory signals, where they become stabilized, translated or rapidly degraded. ELAV1 particularly binds avidly to the AU-rich element in c-fos and interleukin-3 mRNAs. In the case of the c-fos AU-rich element, ELAVL-1 binds to a core element of 27 nucleotides that contain AUUUA, AUUUUA, and AUUUUUA motifs.

ELAVL-1 contains three RNA recognition domains: RNA-BINDING (RRM) 1: Amino acids 20-98; RNA-BINDING (RRM) 2: Amino acids 106-186 and RNA-BINDING (RRM) 3: Amino acids 244-322. Residues 147-154 and 285-292 are the RNA binding regions. HUDSXLRNA (Paraneoplastic encephalomyelitis antigen family signature) is a 6-element fingerprint that provides a signature for the HuD/Elav/Sxl family of RNA-binding proteins.

Variants and Fragments of ELAVL-1

In the context of the present invention the term ELAVL-1 also includes within its scope, variants, derivitives and fragments thereof, in as far as they possess the requisite ability to modulate apoptosis.

Natural variants of ELAVL-1 are likely to comprise conservative amino acid substitutions. Conservative substitutions may be defined, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Suitable fragments of ELAVL-1 will be at least about 5, e.g. 10, 12, 15 or 20 amino acids in length. They may also be less than 100, 75 or 50 amino acids in length. They may contain one or more (e.g. 5, 10, 15, or 20) substitutions, deletions or insertions, including conserved substitutions. A fragment of ELAVL-1 used in the methods of the present invention must possess the requisite activity of being capable of modulating apoptosis.

Measuring Gene Expression

Levels of gene expression may be determined using a number of different techniques.

a) at the RNA Level

Gene expression can be detected at the RNA level. RNA may be extracted from cells using RNA extraction techniques including, for example, using acid phenol/guanidine isothiocyanate extraction (RNAzol B; Biogenesis), or RNeasy RNA preparation kits (Qiagen).Typical assay formats utilising ribonucleic acid hybridisation include nuclear run-on assays, RT-PCR and RNase protection assays (Melton et al., Nuc. Acids Res. 12:7035. Methods for detection which can be employed include radioactive labels, enzyme labels, chemiluminescent labels, fluorescent labels and other suitable labels.

Typically, RT-PCR is used to amplify RNA targets. In this process, the reverse transcriptase enzyme is used to convert RNA to complementary DNA (cDNA) which can then be amplified to facilitate detection.

Many DNA amplification methods are known, most of which rely on an enzymatic chain reaction (such as a polymerase chain reaction, a ligase chain reaction, or a self-sustained sequence replication) or from the replication of all or part of the vector into which it has been cloned.

Many target and signal amplification methods have been described in the literature, for example, general reviews of these methods in Landegren, U., et al., Science 242:229-237 (1988) and Lewis, R., Genetic Engineering News 10: 1, 54-55 (1990).

PCR is a nucleic acid amplification method described inter alia in U.S. Pat. Nos. 4,683,195 and 4,683,202. PCR can be used to amplify any known nucleic acid in a diagnostic context (Mok et al., (1994), Gynaecologic Oncology, 52: 247-252). Self-sustained sequence replication (3SR) is a variation of TAS, which involves the isothermal amplification of a nucleic acid template via sequential rounds of reverse transcriptase (RT), polymerase and nuclease activities that are mediated by an enzyme cocktail and appropriate oligonucleotide primers (Guatelli et al. (1990) Proc. Natl. Acad. Sci. USA 87:1874). Ligation amplification reaction or ligation amplification system uses DNA ligase and four oligonucleotides, two per target strand. This technique is described by Wu, D. Y. and Wallace, R. B. (1989) Genomics 4:560. In the Qβ Replicase technique, RNA replicase for the bacteriophage Qβ, which replicates single-stranded RNA, is used to amplify the target DNA, as described by Lizardi et al. (1988) Bio/Technology 6:1197.

Alternative amplification technology can be exploited in the present invention. For example, rolling circle amplification (Lizardi et al., (1998) Nat Genet 19:225) is an amplification technology available commercially (RCAT™) which is driven by DNA polymerase and can replicate circular oligonucleotide probes with either linear or geometric kinetics under isothermal conditions. A further technique, strand displacement amplification (SDA; Walker et al., (1992) PNAS (USA) 80:392) begins with a specifically defined sequence unique to a specific target.

Primers suitable for use in various amplification techniques can be prepared according to methods known in the art.

Once the nucleic acid has been amplified, a number of techniques are available for the quantification of DNA and thus quantification of the RNA transcripts present. Methods for detection which can be employed include radioactive labels, enzyme labels, chemiluminescent labels, fluorescent labels and other suitable labels.

The detection of nucleic acids encoding ELAVL-1 can be used, in the context of the present invention, to identify early stage apoptosis in cells—a decrease in ELAVL-1 transcripts is associated with the onset of apoptosis. An increase is associated with cell survival and, in particular, is an early response in GM-CSF-mediated inhibition of apoptosis in neutrophils.

b) at the Polypeptide Level

Gene expression may also be detected by measuring the ELAVL-1 polypeptide. This may be achieved by using molecules which bind to the ELAVL-1 polypeptide. Suitable molecules/agents which bind either directly or indirectly to ELAVL-1 in order to detect the presence of the protein include naturally occurring molecules such as peptides and proteins, for example antibodies, or they may be synthetic molecules.

Standard laboratory techniques such as immunoblotting can be used to detect altered levels of ELAVL-1, as compared with untreated cells in the same cell population. An example of a suitable protocol is detailed below:

Aliquots of total protein extracts (40 μg), are run on SDS-PAGE and electroblotted overnight at 4° C. onto nitrocellulose membrane. Immunodetection involved antibodies specific for ELAVL-1, appropriate secondary antibodies (goat, anti-rabbit or goat-anti-mouse: Bio-Rad, Calif., USA) conjugated to horseradish peroxidase, and the enhanced ECL chemiluminescence detection system (Amersham, UK).

Gene expression may also be determined by detecting changes in post-translational processing of polypeptides or post-transcriptional modification of nucleic acids. For example, differential phosphorylation of polypeptides, the cleavage of polypeptides or alternative splicing of RNA, and the like may be measured. Levels of expression of gene products such as polypeptides, as well as their post-translational modification, may be detected using proprietary protein assays or techniques such as 2D polyacrylamide gel electrophoresis.

Monitoring the Onset of Apoptosis

A number of methods are known in the art for monitoring the onset of apoptosis. These include morphological analysis, DNA ladder formation, cell cycle analysis, externalisation of membrane phospholipid phosphatidyl serine and caspase activation analysis. Cell survival may be monitored by a number of techniques including cell cycle analysis and measuring cell viability. Measurements of cell proliferation may be made using a number of techniques including a plaque assay in which adherent cells are plated out in tissue culture plates and left to grow prior to fixing and staining. The number of colonies formed reflects the amount of cell proliferation.

Modifying the Functional Activity of ELAVL-1

The functional activity of ELAVL-1 may be modified by suitable molecules/agents which bind either directly or indirectly to ELAVL-1, or to the nucleic acid encoding it Agents may be naturally occurring molecules such as peptides and proteins, for example antibodies, or they may be synthetic molecules. Methods of modulating the level of expression of ELAVL-1 include, for example, using antisense techniques.

Antisense constructs, i.e. nucleic acid, preferably RNA, constructs complementary to the sense nucleic acid or mRNA, are described in detail in U.S. Pat. No. 6,100,090 (Monia et al), and Neckers et al., 1992, Crit Rev Oncog 3(1-2):175-231, the teachings of which document are specifically incorporated by reference. Suitable antisense molecules may be variants, based on these molecules, which have been chemically modified. For example, the antisense nucleic acids can usefully include altered, often nuclease-resistant, internucleoside bonds. See Hartmann et al. (eds.), Manual of Antisense Methodology (Perspectives in Antisense Science), Kluwer Law International (1999) (ISBN:079238539X); Stein et al. (eds.), Applied Antisense Oligonucleotide Technology, Wiley-Liss (cover (1998) (ISBN: 0471172790); Chadwick et al. (eds.), Oligonucleotides as Therapeutic Agents—Symposium No. 209, John Wiley & Son Ltd (1997) (ISBN: 0471972797).

Other modified oligonucleotide backbones are, for example, phosphorothioates, chiral phosphorothioates, phosphonates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′.

Other modified oligonucleotide backbones for antisense use that do not include a phosphorus atom have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Other methods of modulating gene expression are known to those skilled in the art and include dominant negative approaches as well as introducing peptides or small molecules which inhibit gene expression or functional activity.

RNA-mediated interference (RNAi) is another method for modulating gene expression based on a biological response to double-stranded RNA (dsRNA) resulting in the degradation of homologous mRNA (Dernberg and Karpen, Cell 111:159-162, 2002). Since its description in 1998 (Fire and Mello, Nature 391:806-811, 1998) RNAi has rapidly become a standard experimental tool for targeted destruction of mRNAs in worms, flies, plants and mammals (for review see McManus and Sharp, Nature Genetics 3:737-747, 2002). At present, RNAi is believed to function primarily as a cellular defence mechanism against viruses and transposable elements (Ketting et al, Cell 99:133-141, 1999; Tabara et al, Cell 99:132-132, 1999; see also Elbashir et al, Genes Dev. 15:188-200, 2001). However, RNAi-like processes also appear to be involved in the post-transcriptional regulation of a variety of metazoan developmental processes (reviewed in Ruvkun, Science 294:797-799, 2001).

The introduction of dsRNA to cells triggers the specific degradation of homologous RNAs only within the region of identity with the dsRNA (Zamore et al, 2000). Significantly, RNA interference is initiated when the dsRNA is processed to short 21-23 nt fragments (Zamore et al, Cell 101:25-33, 2000; Bernstein et al, Nature 409:363-366, 2001). In vitro synthesised 21-23 nucleotide dsRNA molecules, called small interfering RNAs (siRNAs), also induce the RNA interference effect (Elbashir et al, Genes Dev. 15:188-200, 2001). These siRNAs are now used routinely in mammalian cells to study the functional consequences of reducing the expression of specific genes (McManus and Sharp, Nature Genetics 3:737-747, 2002). Methods for designing effective siRNAs are described, for example, in http://www.ambion.com/hottopics/rnai.

In addition, changes in events immediately down-stream of ELAVL-1, such as RNA stability, can be used as an indication that a molecule in question affects the functional activity of ELAVL-1.

Modulator Screening Assays

Compounds having inhibitory, activating, or modulating activity can be identified using in vitro and in vivo assays for ELAVL-1 activity and/or expression, e.g., ligands, agonists, antagonists, and their homologs and mimetics.

Modulator screening may be performed by adding a putative modulator test compound to a tissue or cell sample, and monitoring the effect of the test compound on the function and/or expression of ELAVL-1. A parallel sample which does not receive the test compound is also monitored as a control. The treated and untreated cells are then compared by any suitable phenotypic criteria, including but not limited to microscopic analysis, viability testing, ability to replicate, histological examination, the level of a particular RNA or polypeptide associated with the cells, the level of enzymatic activity expressed by the cells or cell lysates, and the ability of the cells to interact with other cells or compounds.

Methods for inducing apoptosis are well known in the art and include, without limitation, exposure to chemotherapy or radiotherapy agents and withdrawal of obligate survival factors (e.g. GM-CSF, NGF) if applicable. Differences between treated and untreated cells indicates effects attributable to the test compound

Myeloid cells die spontaneously in culture although with differing time courses depending on the cell type. Neutrophils in culture apoptose within 24 hours although this can be delayed to over 48 hours in the presence of survival factors. Eosinophil apoptosis is observed over 48 hours with a delay to several days in the presence of survival factors. Macrophages are generally much longer lived. Thus, the ability of a compound to modulate myeloid cell apoptosis can be assessed by monitoring the rate of apoptosis in the presence or absence of the test compound and after the withdrawal of obligate survival factors (e.g. GM-CSF, IL-8, IL-5, G-CSF or BAL) if applicable. Differences between treated and untreated cells indicates effects attributable to the test compound.

Expressing ELAVL-1 in Cells

ELAVL-1 may be expressed in cells by introducing vectors encoding the ELAVL-1 polypeptide.

Particularly useful in the present invention are those vectors that will drive expression of polypeptides from the inserted heterologous nucleic acid (“expression vectors”). These will often include a variety of other genetic elements operatively linked to the protein-encoding heterologous nucleic acid insert, typically genetic elements that drive transcription, such as promoters and enhancer elements, those that facilitate RNA processing, such as transcription termination and/or polyadenylation signals, and those that facilitate translation, such as ribosomal consensus sequences. As will be recognised by those skilled in the art, conditions that permit expression of the polypeptide from these vectors will depend on the type of vector and cell expression system chosen.

Vectors for expressing proteins are known for expression in prokaryotic cells, in yeast cells, typically S. cerevisiae and in mammalian cells and each include the specifc genetic elements for expression in the particular cell type.

Vector-drive protein expression can be constitutive or inducible. Inducible vectors include either naturally inducible promoters, such as the trc promoter, which is regulated by the lac operon, and the pL promoter, which is regulated by tryptophan, the MMTV-LTR promoter, which is inducible by dexamethasone, or can contain synthetic promoters and/or additional elements that confer inducible control on adjacent promoters.

Methods for introducing the vectors and nucleic acids into host cells are well known in the art; the choice of technique will depend primarily upon the specific vector to be introduced and the host cell chosen. Plasmid vectors will typically be introduced into chemically competent or electrocompetent bacterial cells. Vectors can be introduced into yeast cells by spheroplasting, treatment with lithium salts, electroporation, or protoplast fusion. Mammalian and insect cells can be directly infected by packaged viral vectors, or transfected by lipid, chemical or electrical means.

Expression vectors can be designed to fuse the expressed polypeptide to small protein tags that facilitate purification and/or visualization.

For example, proteins can be expressed with a tag that facilitates purification of the fusion protein. Suitable tags and their purification means are known and include poly-his/immobilized metal affinity chromatography, glutathione-S-transferase/glutathione affinity resins, Xpress epitope/detectable by anti-Xpress antibody (Invitrogen, Carlsbad, Calif., USA), myc tag/anti-myc tag antibody, V5 epitope/anti-V5 antibody (Invitrogen, Carlsbad, Calif., USA) and FLAG® epitope/anti-FLAG® antibody (Stratagene, La Jolla, Calif., USA).

For secretion of expressed proteins, vectors can include appropriate sequences that encode secretion signals, such as leader peptides. Expression vectors can also be designed to fuse proteins encoded by the heterologous nucleic acid insert to polypeptides larger than purification and/or identification tags. Useful protein fusions include those that permit display of the encoded protein on the surface of a phage or cell, fusions to intrinsically fluorescent proteins, such as those that have a green fluorescent protein (GFP)-like chromophore, fusions to the IgG Fc region, and fusions for use in two hybrid systems.

ELAVL-1 protein can be expressed and purified from systems such as these for use in methods for detecting molecules which interact with ELAVL-1.

Detecting Molecules which Interact with ELAVL-1

Techniques such as analytical centrifugation, affinity binding studies involving chromatography or electrophoresis can be used to detect molecules which interact directly with ELAVL-1. Those skilled in the art will appreciate that this list is by no means exhaustive. More specifically, it is possible to use ELAVL-1 as an affinity ligand to identify agents which bind to it; labeling ELAVL-1 with a detectable label and using it as a probe to detect apoptotic products in electrophoresis gels; labeling the ELAVL-1 target and using it to probe libraries of genes and/or cDNAs; labeling the ELAVL-1 target and using it to probe cDNA expression libraries to find clones synthesizing proteins which can bind to the target; performing UV-crosslinking studies to identify agents which can bind to the target; using the ELAVL-1 in gel retardation assays which would detect its ability to bind to nucleic acid encoding identified agents; performing footprinting analyses to identify the regions within a nucleic acid to which the target binds. Those skilled in the art will be aware of other suitable techniques and will appreciate that this list is not intended to be exhaustive.

Another technique that allows the identification of protein-protein interactions is immunoprecipitation. An example of a protocol for immunoprecipitation is detailed below:

For immunoprecipitation, lysates from sonicated, Triton X-100-solublized cells (60 μg protein in 100 μl PBS with protease inhibitors) are incubated for 90 min at 37° C. with 500 ng affinity-purified rabbit polyclonal antibodies specific for ELAVL-1, followed by an addition of 10 μl packed protein A/G-agarose beads (30 min, 37° C.: Santa Cruz Biotechnology), vigorous washing of the pellet (10 min at 10000 g, 3×) in PBS, 5% SDS PAGE, and immunodetection with an ELAVL-1-specific mAb.

Another useful technique for identifying interacting protein is the yeast-two hybrid system described, for example in Bartel et al. (eds.), The Yeast Two-Hybrid System, Oxford University Press (1997) (ISBN: 0195109384) the disclosure of which is incorporated herein by reference.

Protein interactions can also be analysed using protein arrays. These may be generated by a range of different techniques which allow proteins to be deposited on a flat surface at different densities. High density protein arrays can be generated using automated approaches similar to those described for DNA arrays (see below). Proteins interacting with ELAVL-1 may be identified by, for example, using ELAVL-1 protein to probe an expression array. Positive interactions could then be detected by the presence of, for example, a labelled antibody or by placing a tag on ELAVL-1. The identity of the interacting protein can be determined by techniques such as mass spectrometry.

Cells

Cells useful in the method of the invention may be from any source, for example from primary cultures, from established cell lines, in organ culture or in vivo. Cell lines useful in the invention include cells and cell lines of haematopoietic origin. Suitable cells include HeLa, U937 (monocyte), TF-1, HEK293 (T), primary cultures of neutrophils or cells having neutrophil characteristics, for example HL60 cells, murine FDCP-1, FDCPmix, 3T3, primary or human stem cells.

Measuring Global Gene Expression

Expression of recombinant ELAVL-1 in cells can be induced using an expression system including any of those described herein.

Determination of expression levels of genes associated with ELAVL-1 will enable the identification of other known or novel genes that play a role in apoptosis.

Regulation of gene activity can be accomplished at a number of levels. Most commonly, regulation is at the transcriptional level—specific transcription factors modulate the expression of subsets of target genes. Post-transcriptional regulation (translational regulation), is determined by the rate and mechanism of RNA processing in the cell, i.e. accumulation, translation and degradation. Subsequent protein-level regulation of genetic activity is accomplished through post-translational modification

A number of individual gene product types whose expression or function is. associated with ELAVL-1 gene expression may be screened for in the present invention. These products include polypeptides and nucleic acids. The expression levels assessed may be absolute levels of production of a particular polypeptide or nucleic acid, or the levels of production of a derivative of any polypeptide or nucleic acid. For example, the invention may be configured to measure the level of expression of a particular mRNA splice variant, or the amount present of a phosphorylated derivative of a particular polypeptide.

Where it is desired to monitor the levels of expression of a known gene product, conventional assay techniques may be employed, including nucleic acid hybridisation studies and activity-based protein assays. Kits for the quantitation of nucleic acids and polypeptides are available commercially.

Where the gene product to be monitored is unknown, however, methods are employed which facilitate the identification of the gene product whose expression is to be measured. For example, where the gene product is a nucleic acid, arrays of oligonucleotide probes may be used as a basis for screening populations of mRNA derived from cells.

a) Arrays

Gene Arrays of oligonucleotides specific to gene sequences archived in public domain databases, such as GenBank, are available commercially from a number of suppliers (such as Incyte Genomics, USA). Examples of such commercial arrays are in the form of either nucleotides spotted onto a membrane filter (such as nitrocellulose), or a solid support (such as glass). Commercial Gene Arrays are used to profile the patterns of gene expression which are associated with the process of apoptosis in neutrophils, and other cell types.

Gene Arrays can additionally be constructed specifically, by spotting nucleotide sequences derived from cDNA clones generated from novel libraries or from cDNA clones purchased commercially. Such arrays allow the expression profiling of proprietary and/or novel nucleotide sequences.

Gene Arrays are additionally constructed by commercial sources (e.g. Genescreen), by spotting nucleotide sequences derived from cDNA clones generated from novel libraries or from cDNA clones purchased commercially. Such arrays allow the expression profiling of proprietary and/or novel nucleotide sequences.

Many of the cDNA sequences or EST (expressed sequence tag) sequences deposited in the public domain databases are derived from a restricted set of tissue types, such as liver, brain and foetal tissue. The cloning of in-house cDNA libraries which are focused to specific cellular events, such as ROS-mediated apoptosis offers the possibility to identify, clone and characterise novel genes which are associated with this process. Similarly, the cloning of in-house cDNA libraries which are focused to specific tissue types, such as the neutrophil, offers the possibility to identify, clone and characterise novel genes whose expression is restricted to this cell type. Libraries (cDNA) constructed using a physical subtraction, such as the ClonTech ‘Select’ SSH method (suppression hybridisation) and novel modifications of such, as described, allow the selective cloning of genes whose expression is differentially regulated in the process or cell type being studied. Gene Array technology is combined with SSH cDNA libraries to identify false-positives and further focus on truly differentially expressed genes. Clones from each SSH library constructed are picked, cultured and archived as glycerol stocks. The cDNA inserts contained within individual plasmid clone are PCR amplified and spotted onto in-house arrays. Differential expression is confirmed using hybridisation with a radiolabelled probe generated from the mRNA used for each reciprocal subtractions.

Arrays of nucleic acids may be prepared by direct chemical synthesis of nucleic acid molecules. Chemical synthesis involves the synthesis of arrays of nucleic acids on a surface in a manner that places each distinct nucleic acid (e.g., unique nucleic acid sequence) at a discrete, predefined location in the array. The identify of each nucleic acid is determined by its spatial location in the array. These methods may be adapted from those described in U.S. Pat. No. 5,143,854; WO90/15070 and WO92/10092; Fodor et al. (1991) Science, 251: 767; Dower and Fodor (1991) Ann. Rep. Med. Chem., 26: 271.

In a preferred aspect of the invention, arrays of nucleic acids may be prepared by gridding of nucleic acid molecules. Oligonucleotides may be advantageously arrayed by robotic picking, since robotic techniques allow the most precise and condensed gridding of nucleic acid molecules; however, any technique, including manual techniques, which is suitable for locating molecules at discrete locations on a support, may be used.

The gridding may be regular, such that each colony is at a given distance from the next, or random. If molecules are spaced randomly, their density can be adjusted to statistically reduce or eliminate the probability of overlapping on the chosen support.

Apparatus for producing nucleic acid microarrays is available commercially, for example from Genetix and Genetic Microsystems. Moreover, pre-prepared arrays of nucleic acid molecules are available commercially, for example from Incyte Genomics Inc. (Human LifeGrid™). Such arrays will comprise expressed sequence tags (ESTs) representative of most or all the genes expressed in a cell or organism, thus providing a platform for the screening of mRNA populations from multiple ROS-treated cells.

Samples for mRNA population analysis may be isolated and purified by any suitable mRNA production method; for example an RNA isolation kit is available from Stratagene.

In addition, where the gene product is a polypeptide, arrays of antibodies may be used as a basis for screening populations of polypeptides derived from cells. Examples of protein and antibody arrays are given in Proteomics: A Trends Guide, Elsevier Science Ltd., July 2000 which is incorporated by reference.

b) 2D PAGE

For the monitoring of unknown polypeptide gene products, separation techniques such as 2 dimensional gel electrophoresis are employed. 2D PAGE typically involves sample preparation, electrophoresis in a first dimension on an immobilised pH gradient, SDS-PAGE electrophoresis in a second dimension, and sample detection. Protocols for 2D PAGE are widely available in the art, for example at http://www.expasy.ch/ch2d/protocols/, the contents of which as of 30.11.2001 are incorporated herein by reference. Samples for 2D PAGE may be prepared by conventional techniques. In the case of the present invention, HeLa cells transfected with ELAVL-1 are grown in a suitable medium, such as RPMI 1640 containing 10% foetal calf serum (FCS). The suspension is transferred into a tube and the cells are centrifuged at 1000 g for 5 minutes. Supernatant is discarded and the cells are washed with RPMI 1640 without FCS. After centrifugation and removal of RPMI 1640, 0.8×10⁶ cells are mixed and solubilised with 60 μl of a solution containing urea (8 M), CHAPS (4% w/v), Tris (40 mM), DTE (65 mM) and a trace of bromophenol blue. The whole final diluted HeLa sample is loaded on the first dimensional separation.

The method of the present invention advantageously employs a step of establishing a reference expression level for the gene products being investigated. This can be carried out by using un-transfected HeLa cells to serve as a standard for one or more subsequent assays; or it may be an integral part of every assay. For example mRNA or polypeptide populations from HeLa transfected and untransfected cells may be assessed simultaneously on a nucleic acid array or by 2D PAGE, and changes in expression patterns identified by direct comparison.

Analysis of 2D PAGE results, using appropriate software where necessary, reveals polypeptides of interest which may be isolated, sequenced and used to identify genes encoding them.

Translational Regulation

Regulation of gene activity can be accomplished at a number of levels. Most commonly, regulation is at the transcriptional level—specific transcription factors modulate the expression of subsets of target genes. Post-transcriptional regulation, (translational regulation), is determined by the rate and mechanism of RNA processing in the cell, i.e. accumulation, translation and degradation. Subsequent protein-level regulation of genetic activity is accomplished through post-translational modification.

Translational regulation of gene activity is a feature of a variety of cellular processes, notably differentiation, proliferation, protection from external damage and regulation of metabolic pathways. Regulation at this level ensures the rapid and efficient production of critical gene products without the time-lag typically associated with RNA synthesis and processing. Specific control is achieved in the vast majority of known cases by changes in the translational initiation rate. Other mechanisms include alterations in translational elongation rate, or alteration of mRNA stability (through alterations of poly(A) tail length). The rate of initiation of translation can be altered by changes in the protein composition of the initiation complex, or by association of trans-acting factors (protein or RNA) with the target RNA.

An increasing number of reports suggest that translational regulation of gene expression is a feature of apoptosis, particularly following caspase activation (for review see Clemens et al. Cell Death Differ. 2000 7(7): 603-15). An early target of activated caspases is the eIF4G (elongation initiation) protein, which is required for cap-dependent protein synthesis. Significantly, the anti-apoptotic function of Ras has also been shown to be mediated, in part at least, by modulation of the cap-dependent translation initiation apparatus (Polunovsky et al., J Biol Chem. 2000 275(32): 24776-80.). Specifically, the mRNA-cap binding protein repressor, 4EBP1, has proapoptotic activity, which is strictly dependent on its ability to sequester the cap binding protein eIF4E, thus preventing assembly of the activation complex. Ras, through the Akt and FRAP/mTOR kinases, can activate the cap-dependent protein synthesis apparatus, thereby inhibiting apoptosis.

Methods for detecting mRNA Stability

a) One method which can be used to identify mRNAs whose stability and/or translational status is altered by ELAVL-1 expression comprises 3 steps as follows. This assay is based on the assumption that translationally-inactive mRNAs are present in the cytoplasm as mRNPs, whereas actively translated mRNAs are contained within polysomes (Keene J P, Proc. Natl. Acad. Sci. (USA) 98(13): 7018-7024 (2001)). To isolate and identify mRNAs selectively mobilized from RNPs to polysomes, cDNA probes are prepared from RNP and polysome fractions, and hybidised against cDNA microarrays. Altering the expression of ELAV alters the subset of mRNAs present within the cell as RNPs. By comparing cells subjected to different ELAV-modifying regimes, mRNAs whose stability and/or translational status altered by ELAV can be identified.

i) Cell Harvest and Extraction

Cells in suspension cultures are poured into 50 mL conical tubes containing one fifth of a volume of frozen, crushed PBS (: 0.14 M NaCl, 5 mM Na2HPO4, and 1.5 mM KH2PO4, pH 7.2) and a final concentration of 100 mg Cycloheximide per milliliter. The chilled cultures are centrifuged at 1000 g for 5 min at 4° C. Cell pellets are washed with 10 mL ice-cold PBS and centrifuged again at 1000 g for 5 min at 4° C. The cell pellet is resuspended in 750 uL LSB (200 mM Tris (pH 7.4-7.5), 100 mM NaCl, 30 mM MgC12) at 4° C. Cells are allowed to swell on ice for 3 min. Detergent buffer (250 ul), (1.2% (v/V) Triton N-101 in LSB) is added and the solution is immediately transferred to a 7 mL Dounce homogenizer on ice. The cells are lysed with eight strokes of the homogenizer. The solution is transferred to an ice-chilled 15 mL Falcon tube and centrifuged at 4° C., 10,000 g, for 1 min. The supernatant is then transferred to a 1.5 mL microfuge tube containing 100 uL if 10 mg heparin per milliliter and 1.5 M NaCl in LSB. Cell lysates are immediately layered onto sucrose gradients for centrifugation.

ii) Sucrose Gradient Centrifugation and Polysome Fractionation

Sucrose solution, 5.5 mL of 1.5 M, is added to the bottom of a Beckman 14×95-mm tube (Part No. 331374, Beckman Instruments, Fullerton, Calif.). A further 5.5 mL of 0.5 M sucrose solution is layered onto this, without disturbing the interface of the two gradients.

The top of the tube is sealed with parafilm, and the tube is stored at 4° C. for 6-24 hr. The cell extracts are carefully layered onto the top of the sucrose gradient without disturbing the interface, and then centrifuged in a Beckman SW 40 rotor at 4° C., at 36,000 rpm for 110 min.

Centrifuged gradients are fractionated into 12 1-mL fractions using an ISCO Density Gradient Fractionator at a flow rate of 3 mL/min. The polysome profile is monitored via UV absorbance at 254 nm, and the fractions are collected into 1.5 mL microfuge tubes containing 100 uL 10% SDS. Each sample is digested with 11 uL proteinase K solution at 37° C. for 30 min.

iii) Reverse Transcription and Array Hybridization

RNA is reverse transcribed as described and hybridized to Incyte Lifegrid filters. Array data is captured using a Cyclone phosphoimager (HP Biosciences). Differential composition of polysome/RNP fractions are determined by comparison of reference and treatment group preparations.

b) A second method relies on immunoprecipitation of Endogenous mRNP Complexes from Cell Lysates. Where expression of ELAVL-1 expression is altered, the subset of mRNAs present within the cell as RNPs whose translational status is impacted by ELAV maybe identified.

Polyclonal sera reactive with ELAV are obtained from Jack D. Keene (Department of Microbiology, Duke University Medical Center, Durham, N.C. 27710) or obtained commercially.

i) Cells are removed from tissue culture plates with a rubber scraper and washed with cold PBS. The cells are resuspended in approximately two pellet volumes of polysome lysis buffer containing 100 mM KCl, 5 mM MgCl2, 10 mM Hepes, pH 7.0, and 0.5% Nonidet P-40 with 1 mM DTT, 100 units/ml RNase OUT (GIBCO/BRL), 0.2% vanadyl ribonucleoside complex (GIBCO/BRL), 0.2 mM PMSF, 1 mg/ml pepstatin A, 5 mg/ml bestatin, and 20 mg/ml leupeptin added fresh at time of use. The lysed cells are then frozen and stored at −80° C. At the time of use, the cell lysate is thawed and centrifuged at 16,000×g in a tabletop microfuge for 10 min at 4° C. The mRNP cell lysate typically contains approximately 50 mg/ml total protein.

ii) For immunoprecipitations, Protein-A Sepharose beads (Sigma) are swollen 1:5 V/V in NT2 Buffer (50 mM Tris, pH 7.4/150 mM NaCl/l mM MgCl2/0.05% Nonidet P-40) supplemented with 5% BSA. A 300-μl aliquot of the 1:5 V/V preswollen protein-A bead slurry is used per immunoprecipitation reaction and incubated overnight at 4° C. with excess immunoprecipitating antibody (typically 5-20 μl depending on the reagent). The antibody-coated beads were washed with ice-cold NT2 buffer and resuspended in 900 μl of NT2 buffer supplemented with 100 units/ml RNase OUT/0.2% vanady/ribonucleoside complex/1 mM DTT/20 mM EDTA. The beads are briefly vortexed, and 100 μl of the mRNP cell lysate is added and immediately centrifuged and a 100-μl aliquot removed to represent total cellular RNA. The immunoprecipitation reactions are mixed at room temperature for 2 hours and then washed 4 times with ice-cold NT2 buffer followed by 2 washes with NT2 buffer supplemented with 1 M urea. Washed beads are resuspended in 100 μl NT2 buffer supplemented with 0.1% SDS and 30 μg proteinase K incubated for 30 min in a 55° C. water bath, phenol-chloroform-isoamylalcohol extracted and ethanol precipitated.

iii) Reverse Transcription and Array Hybridization

RNA is reverse transcribed as described and hybridized to Incyte Lifegrid filters. Array data is captured using a Cyclone phosphoimager (HP Biosciences). Differential composition of RNP fractions following altered expression of ELAV are determined by comparison of reference and treatment group preparations.

Transcription Factors

The function and context of several of the transcription factors and enhancers for which binding sites have been identified in the ELAVL-1 promoter are associated with growth factor signalling, PI3kinase activation, haematopoietic cell differentiation (especially granulocyte lineages), cell survival and also redox regulation, as described:

The MEF2 genes are members of the MADS gene family (named for the yeast mating type-specific transcription factor MCM1, the plant homeotic genes ‘agamous’ and ‘deficiens’) and the human serum response factor a family that also includes several homeotic genes and other transcription factors, all of which share a conserved DNA-binding domain. They have been associated with cellular differentiation (muscle; Yu et al. Genes Dev. 6: 1783-1798, 1992), growth factor-induction (Pollock and Treisman Genes Dev. 5: 2327-2341, 1991.). MEF2 are also induced via the p38 MAP kinase cascade, in neurons where they are critical for survival (Mao et al. Science 286: 785-790, 1999).

GATA-1 is a haematopoetic transcription factor. GATA1 and friend of GATA1 (FOG) are each essential for erythroid and megakaryocyte development. FOG, a zinc finger protein, interacts with the amino (N) finger of GATA1 and cooperates with GATA1 to promote differentiation.

C/EBP is a CCAAT/enhancer-binding protein in the hematopoietic system, CEBPA is exclusively expressed in myelomonocytic cells. It is specifically upregulated during granulocytic differentiation. No mature granulocytes are observed in Cebpa-mutant mice, whereas all the other blood cell types are present in normal proportions.

The commitment of multipotent cells to particular developmental pathways requires specific changes in their transcription factor complement to generate the patterns of gene expression characteristic of specialized cell types. C/EBPs and specific Ets family members, together with GATA-1, are important for eosinophil lineage determination (McNagny K M et al. EMBO J 1998 Jul. 1;17(13):3669-80). GATA-1 and C/EBPbeta synergistically transactivate the promoter of an eosinophil-specific granule protein gene and FOG may act as a negative cofactor for the eosinophil lineage (Yamaguchi Y et al. Blood 1999 Aug. 15;94(4):1429-39). FOG is a repressor of the eosinophil lineage, and C/EBP-mediated down-regulation of FOG is a critical step in eosinophil lineage commitment. Maintenance of a multipotent state in hematopoiesis is achieved through cooperation between FOG and GATA-1 (Querfurth E et al. Genes Dev 2000 Oct. 1;14(19):2515-25).

The transcription factors cFos and cJun (or AP-1) mediate their effects through binding as homo- or heterodimers to the AP-1 binding site in mammalian promoters. Fos and Jun family proteins regulate the expression of a myriad of genes in a variety of tissues and cell types (Chinenov Y and Kerppola T K Oncogene Arp. 30; 20(19):2438-52). Like NfkappaB, AP-1 is a redox-sensitive transcription factor. The development of an oxidant/antioxidant imbalance in lung inflammation may activate redox-sensitive transcription factors such as nuclear factor-kappa B (NF-kappa B) and activator protein-1 (AP-1), which regulate the genes for proinflammatory mediators and protective antioxidant genes. GSH, a ubiquitous tripeptide thiol, is a vital intra- and extracellular protective antioxidant against oxidative stress, which plays a key role in the control of proinflammatory processes. The promoter regions of the human gamma-GCS subunits contain AP-1, NF-kappa B, and antioxidant response elements and are regulated by oxidants, growth factors, inflammatory cytokine tumor necrosis factor-alpha (TNF-alpha), and anti-inflammatory agent (dexamethasone). Fine tuning between the redox GSH levels and the activation of transcription factors may determine the balance of transcription for proinflammatory and antioxidant gamma-GCS genes in inflammation (Rahman IBiochem Pharmacol 2000 Oct. 15;60(8):1041-9). AP-1 is down-stream of and in part mediates the effects of the growth-factor signal transduction molecule phosphatidylinositol-3 kinase (PI-3 K). TPA, EGF and insulin induce PI-3 K activity in JB6 cells. The induced PI-3 K activity was blocked by a dominant negative mutant of PI-3 K, and by wortmannin or LY294002. Blocking of PI-3 K activity by these inhibitors also blocked TPA- or EGF-induced AP-1 activity and cell transformation (Dong Z et al Anticancer Res 1999 September-October 1999;19(5A):3743-7).

Methods for Detecting Transcription from a Promoter Sequence

Transcription from the ELAVL-1 promoter sequence can be detected using a nucleic acid construct comprising the ELAVL-1 promoter sequence operably linked to a reporter gene. A “reporter gene” is a gene which is incorporated into an expression vector and placed under the same controls as a gene of interest to express an easily measurable phenotype. A number of suitable reporter genes are known whose expression may be detectable by histochemical staining, liquid scintillation, spectrophotometry or luminometry. Many reporters have been adapted for a broad range of assays, including calorimetric, fluorescent, bioluminescent, chemiluminescent, ELISA, and/or in situ staining. Suitable reporter systems are based on the expression of enzymes such as chloramphenicol acetyltransferase (CAT), b-galatosidase (b-gal), b-glucuronidase, alkaline phosphatase and luciferase. More recently, a number of reporter systems have been developed which are based on using Green fluorescent proteins (GFP) or various derivatives or mutant forms including EGFP. Reporter genes and detection systems are reviewed by Sussman in The Scientist 15[15]:25, Jul. 23, 2001 which is incorporated by reference.

One method for identifying a compound that modulates expression from the ELAVL-1 promoter is outlined below. In this method GM-CSF is used to activate transcription from the ELAVL-1 promoter but other known or test compounds could be used.

Primers to amplify the promoter region are selected, using the Primer Designer facility of the GeneTool Lite software (Biotools Inc), which have minimal internal stability and annealing temperatures of 60° C. A PCR reaction is carried out to amplify the promoter sequence from genomic DNA. Amplimers are gel purified using the Qiaquick gel isolation kit (Qiagen, cat. 28706) and ligated to pCDNA3.1 using the Topo-TA cloning kit (Invitrogen, cat. 45-0005) according to the manufacturers instructions. Ligated DNA is transformed to E.coli (Top10). Transformants are selected for plasmid DNA preparation and sequence analysis.

Plasmid DNA is prepared using either the Qiagen miniprep (cat. 27106) or midiprep (cat. 12643) kits as described by the manufacturer. Insert orientation is determined by PCR with ELAV-specific reverse primer and vector-specific forward primer (T7 primer). Plasmid miniprep DNA (100 ng to 5 μg) is sent to MWG Biotech or Lark Technologies for contract sequencing.

U937 cells are transfected with 10 μg of an EGFP reporter construct (pEGFP, Clontech), containing a genomic fragment driving the expression of the EGFP gene (Elav-EGFP). The fragment includes the putative ELAVL-1 promoter region. Cells are transfected by the calcium phosphate method. Transfection of the pEGFP vector without the ELAV genomic fragment is used as a negative control whereas a construct containing the CMV promoter serves as a positive control.

U937 cells containing either pEGFP or ELAV-EGFP are treated with GM-CSF (50 Units) either in the presence or absence of gliotoxin (0.1 μg/ml). At the indicated periods of time, cells are examined for EGFP expression by flow cytometric analysis using a FacsCalibre (Becton Dickinson). Cells are considered positive for EGFP expression when the FL1 signal is greater than the background signal generated by either pEGFP or untreated ELAV-EGFP. All values are corrected for transfection efficiency by standardization against β-gal activity, derived from the cotransfected plasmid pSV β-gal (Promega).

An increase in fluorescence is observed in U937 cells transfected with ELAV-EGFP when cultured in the presence of GM-CSF indicating that promoter activity is induced when cells are incubated in the presence of GM-CSF. Treating the cells with GM-CSF in the presence of gliotoxin attenuates this induction. Similarly, there is no increase in fluorescence when U937 cells transfected with pEGFP are treated with GM-CSF.

Single Nucleotide Polymorphisms (SNPs)

Single nucleotide polymorphisms (SNPs) are single base pair positions in genomic DNA at which different sequence alternatives (alleles) exist in normal individuals in a population. SNPs may responsible for variations between individuals, including variations which predispose an individual to a disease or cause it. Approximately half of all coding sequence SNPs result in synonymous (i.e. silent) codon changes. Even those these SNPs may have no effect on protein function, they are potentially useful for tracking other variations nearby as adjacent stretches of DNA tend to be inherited together (‘linkage disequilibrium’). Informative SNPs accelerate the identification of disease genes by allowing researchers to look for associations between a disease and specific SNPs in a population. SNP distributions are informative for drug response and allow stratification of populations for particular therapy or drug treatment regimens.

The invention is further described, for the purposes of illustration only, in the following examples.

### EXAMPLES

Bioinformatic Sequence Analysis Tools

DoubleTwist (www.doubletwist.com) tools were used to analyse the target sequences retrieved from Genbank. The DoubleTwist suite incorporates a number of research agents to generate computational analysis outputs using algorithms that search multiple gene, protein, and patent databases for information about query sequences. These tools access the DoubleTwist annotated databases and all published information about the query sequences. For the purpose of this study the following agents were used: Perform Comprehensive Sequence Analysis; Retrieve Assembled ESTs; Retrieve and Analyse Human Genome.

The Comprehensive Sequence Analysis agent uses the BLAST2N, BLAST2X, TBLAST2N, and BLAST2P algorithms to search the following databases: SwissProt; NR-Nuc; NR-Pro; dbEST; PDB; PAT; PATaa; HTG; Genbank's cumulative nightly nucleotide and protein database updates; and Myriad Genetics ProNet database. Additionally the Blimps and Blkprob algorithms are used to search the Blocks+database. This agent provides information about functional protein identities and similarities, DNA identities and similarities, patented sequences, protein domains, structural identities and similarities, and genomic DNA identities and similarities.

The Assembled ESTs agent (Human) identifies matching EST clusters derived from the DoubleTwist Gene Indices. The Gene Indices are collections of assembled EST and mRNA sequences derived by, screening out non-informative sequences (such as vector and ribosomal sequences), clustering the remaining sequences, first by matching pairs for overlapping bases, then by sub-dividing into gene variants (subclusters); aligning the sequences in each cluster, and deriving a consensus sequence for each cluster and subcluster. The sequence collection is therefore checked and statistically corrected for many sequencing and cloning errors such as orientation, chimerism, and contamination. DoubleTwist's interactive data-mining tool Cluster Viewer was used to visualise the alignments.

The “Analyse Human Genome” agent also uses a proprietary DoubleTwist genome database derived from public data. Genomic sequences that are at least 15 kiilobases in length are obtained from Genbank's Genomic Sequences Primate (GB PRI) division. Unfinished human genomic sequences are obtained from Genbank's High Throughput Genomic (HTG) Sequences division. The data is annotated by splitting the HTG sequences phase 0, 1, and 2 into component fragments while maintaining the GB PRI sequences intact. Sequence contamination, from vector, bacterial, yeast or mitochrondrial sequences are masked and the Repeat Masker program (http://repeatmasker.genome.washington.edu/cgi-bin/R2_req.pl) is used to mask repetitive elements and regions of low complexity. The GrailEXP, FGENESH and Genscan algorithms are then employed to predict coding regions, introns and exons. The Halfwise algorithm is used to match predicted coding regions with models from the Pfam database. The Unigene database and the DoubleTwist Human Gene Index are further searched for DNA similarities using the BLASTN algorithm and the NR Pro database is searched, using BLASIX, for similar proteins.

The Double Twist Genomic Viewer, an interactive data mining and visualization tool was used to examine the output from the Genome Analysis agent.

The GeneTool suite from BTI (BioTools Inc) was used for sequence analysis, ClustalW (http://www.ebi.ac.uk/clustalw/) was used for creating multiple alignments. “Translate Tool” at Expasy (htto://www.expasy.ch/tools/dna.ht was used to translate nucleotide sequences to protein sequences. ORF finder at the NCBI (http://www.ncbi.nlm.nih.gov/gorf/gorf.html) was used to find all open reading frames of a selectable minimum size.

### EXAMPLE 1

### Gene Expression and Cluster Analysis of Neutrophil Apoptosis and Survival; ELAV-Like 1 is Identified by Association, as a Modulator of Apoptosis and Cell Survival

A model system for the identification of early-regulated genes in apoptosis of human primary neutrophils is described in our co-pending applications WO 01/46469 and WO 02/04657.

Isolation and Culture of Primary Human Neutrophils

Whole blood (2-50 ml) is taken from normal healthy volunteers by venepuncture. Coagulation is prevented by the use of sodium citrate. A 6% dextran (mol wt 509,000; Sigma) saline solution is added in 1:4 ratio to whole blood and the erythrocytes allowed to sediment for 45 minutes at 22° C. The buffy coat is then under-layered with 5 ml Ficoll-Paque (Pharmacia LKB Biotechnology) and centrifuged (300 g, 30 min) to pellet granulocytes and erythrocytes (Boyum, 1968). The pellet is resuspended in 1 ml cell culture tested water (Sigma) for 40 sec., followed by the addition of 14 ml Hanks buffer (Sigma) and centrifuged (300 g, 10 min.). This lysis step is repeated to ensure removal of all erythrocytes. The remaining pellet is resuspended in RPMI 1640 supplemented with 10% foetal calf serum (Sigma), L-glutamine (2 mM), penicillin (100 U/ml; Sigma), streptomycin (100 μg/ml; Sigma) and amphotericin B (2.5 μg/ml; Sigma). Cell number and viability is checked using trypan blue exclusion (Boyum, (1968) Scand J Clin Lab Invest Suppl; 97:77-89).

Isolated neutrophils are maintained at a density of 2×10⁶/ml in RPMI 1640 supplemented with 10% foetal calf serum (Sigma). Further additions to the medium included L-glutamine (2 mM), penicillin (100 U/ml), streptomycin (100 μg/ml) and amphotericin B (2.5 μg/ml) (Sigma). Cells are incubated at 37° C. in a humidified CO₂ (5%) incubator. As described in by Haslett (Clinical Science 83, pp 639-648, 1992), WO 01/46469 and WO 02/04657, upon culture in a serum-containing cell culture medium these neutrophils undergo spontaneous apoptosis.

Dose Responsiveness of the Anti-Apoptotic Effect of GM-CSF.

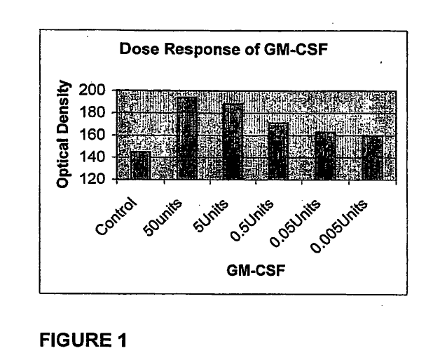

Primary human neutrophils are isolated and purified from peripheral blood of normal healthy individuals. Neutrophils are resuspended in serum containing culture medium together with various amounts of GM-CSF at a density of 2×10⁶/ml, with 100 μl plated into a 96 well plate and cultured for 18 h at 37° C. After this time 10 μl of MTT (5 mg/ml) is added to the cultures and incubated for a further 4 h at 37° C. before solubilisation of the purple coloured formazan with acidic isopropanol. Optical densities are read at 570 nm using a plate reader. FIG. 1 shows there is a direct correlation between survival and concentrations of GM-CSF added to the culture medium.

For subsequent experiments neutrophils are resuspended in serum containing culture medium containing 5 U/ml of GM-CSF.

Fungal Metabolite Gliotoxin Blocks GM-CSF Inhibition of Neutrophil Apoptosis.

This describes the identification of an inhibitor for the GM-CSF mediated inhibition of neutrophil apoptosis. The use of this inhibitor allows us to focus in on the specific biochemical events mediating the GM-CSF survival events. In turn one is able to remove some of the noise associated GM-CSF treatment.

Primary human neutrophils are isolated and purified from peripheral blood of normal healthy individuals. Neutrophils are resuspended in serum containing culture medium containing 5 U/ml of GM-CSF at a concentration of 2×10⁶/ml. Also added to the culture mix is either 0.1 μg/ml of the fungal metabolite Gliotoxin or its inactive analogue bis-Dethio-bis (Methylthio) Methyl Gliotoxin, with 100 μl/well plated into a 96 well plate and culture at 37° C. commenced. After the indicated time, 10 μl of MTT (5 mg/ml) are added to the cultures and incubated for a further 4 h at 37° C. before solubilisation of the purple coloured formazan with acidic isopropanol. Optical densities are read at 570 nm using a plate reader.

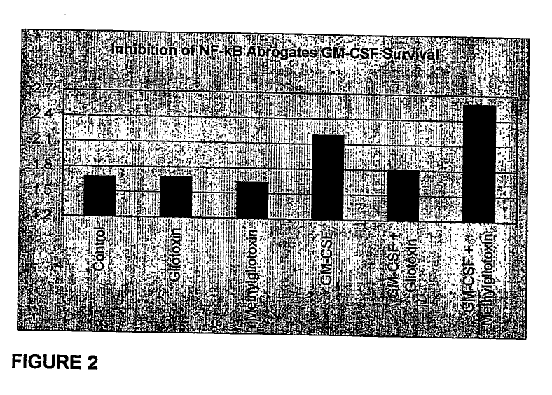

FIG. 2 demonstrates that gliotoxin effectively blocks the GM-CSF inhibition of neutrophil apoptosis. This blocking effect is not seen when the inactive analogue of gliotoxin, methylgliotoxin is added with GM-CSF. No increased neutrophil apoptosis is seen with the addition of gliotoxin alone to isolated neutrophils demonstrating that the effect is specific to and limited to a reversal of the protective effects of GM-CSF.

Commercial microarrays are used to measure global gene expression associated with neutrophil apoptosis, GM-CSF inhibition of neutrophil apoptosis, and the inhibition of this effect using the fungal metabolite Gliotoxin. In control experiments, an inactive analogue of Gliotoxin, Methyl Gliotoxin is used. Analysis of such microarray results identifies genes whose expression pattern changes (either upregulation or down-regulation) in an association with a measurable apoptotic phenotype.

Total RNA Isolation

Primary human neutrophils are isolated and purified from peripheral blood of normal healthy individuals using standard techniques. Neutrophils are resuspended in serum containing culture medium together with GM-CSF (50 U) at a concentration of 2×10⁶/ml, and cultured for 0 h (control), 2 h, 4 h and 6 h at 37° C. Total RNA is then prepared from both groups using acid phenol/guanidine isothiocyanate extraction (RNAzol B; Biogenesis), or RNeasy RNA preparation kits (Qiagen). Any contaminating genomic DNA is removed by DNase treatment (DNase I, Gibco-BRL). RNA is also prepared from neutrophil cells following treatment (for the time indicated in hours) with GM-CSF (50 units/ml), Gliotoxin (10 μM) or MethylGliotoxin (10 μM). RNA is also prepared from neutrophils that have not been exposed to drug (i.e. as an untreated control). RNA is prepared from these cells using two sequential extractions with RNAzol B.

Measurement of Global Gene Expression by ‘Microarray’

The process of microarraying can be used to profile gene expression of thousands of genes simultaneously. The microarray process is described both for the use of Human LifeGrid™ microarray filters and can be separated into three parts: the filter, the hybridisation of radiolabelled cDNA probe, and the detection and quantitation of the microarray results.

The Microarray Filter

This example describes the use of the Human LifeGrid™ microarray filters obtained from Incyte Genomics (USA). These filters contain cDNA probes representing approximately 8,400 human mRNAs.

Hybridisation of Radiolabelled cDNA Probes.

This example describes the synthesis of a radiolabelled cDNA from total cellular mRNA. The labeled cDNA is used to ‘probe’ DNA fragments, which have been immobilised on to a filter membrane, by complementary hybridisation.

Methodology is as described by manufacturer, for Human LifeGrid™ arrays. Essentially, total cellular RNA (1 μg to 20 μg) or polyA+mRNA (100 ng to 5 μg) is incubated with an oligo (dT) primer. Primed RNA is reverse transcribed to first strand cDNA in a reaction containing M-MLV reverse transcriptase (RT; alternatively Superscript II is used (Life Sciences)), RT buffer, dNTPs and [α-³³P] dCTP (2000-4000 Ci/mmol) at 42° C. for 1 to 5 hours. Unincorporated nucleotides are removed using spin-columns and the labeled probe stored at −80° C. until required.

Labeled probes may also be generated from cDNA, genomic DNA or PCR products. In each case a random primed labeling procedure can be used, for example the Ready-Prime Labeling kit (APBiotech), applied as per manufacturers instructions.

Radiolabelled cDNA probe is hybridised to DNA fragments immobilised onto a membrane (typically a nylon or nitrocellulase filter).

Methodology is as described by manufacturer, for Human LifeGrid™ arrays. Essentially, membrane filters are pre-hybridised in hybridisation buffer (5 to 20 ml) at 42° C. for 2 to 16 h using a hybridisation oven (Hybaid). Following pre-hybridisation, the labeled cDNA probe is added to fresh hybridisation buffer (5 to 20 ml) and hybridised at 42° C. for 14 to 16 h. Following hybridisation, the hybridisation mix is removed and the filters washed with 2×SSC buffer at RT for S min., twice with 2×SSC, 1% SDS buffer at 68° C. for 30 min. and twice with 0.6×SSC, 1% SDS buffer at 68° C. for 30 min.

Detection and Quantitation of the Microarray Results.

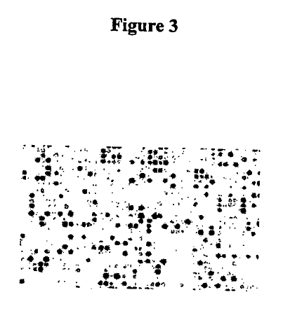

This example describes the use of a STORM Phosphoimager to quantitatively image positive signals across the filter arrays. Hybridised filters are wrapped in plastic wrap (Saran) and exposed to a Low-Energy Phosphoimaging screen (Molecular Dynamics). The screen is then placed on the phosphoimager and the gel image captured by scanning at a resolution of 50 microns (See FIG. 3).

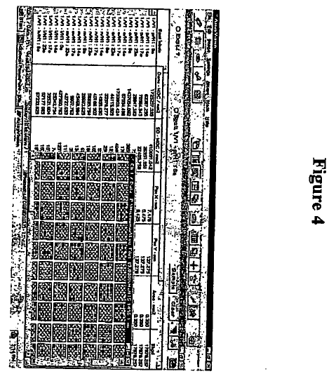

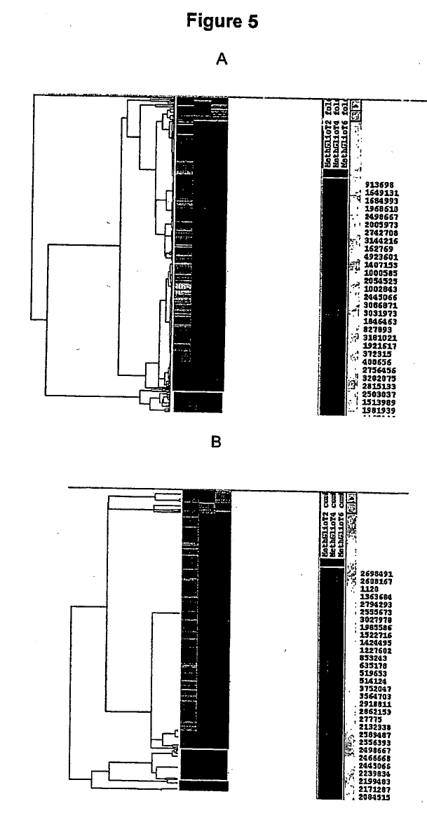

The captured image file is then analysed using software such as Array Vision (Imaging Research Inc.; See FIG. 4). In this example we implement analysis with ArrayVision v5.1. This program contains facilities for spot detection and quantification, and background detection and quantification. This data is then exported to a text file for further analysis. A variety of data fields are exported from the ArrayVision analysis, including; Spot Label, Position, Density, Background, and particularly, Background subtracted density (sDens) and signal/noise ratio (S/N). In this example, the exported text file is up-loaded to an SQL-7.0 database, to populate a table containing array data from all experiments. As the data is imported to the database, a Normalisation factor is calculated and the sDENs values modified accordingly. This Normalised data is stored in a newly created column within the table. The Normalisation factor facilitates accurate comparison between datasets. A number of different calculations may be used. A normalization factor may be derived from Linear Regression calculated by reference to housekeeping genes. Alternatively, the Global Mean is calculated as the average of the sDens values across all of the arrays to be compared and a normalisation factor is then derived by division of the overall spot density with the Global Mean value. Spot density values (individual sDens) are then corrected by multiplying across all values with the normalisation factor. In a similar approach a Global Geometric Mean normalization factor may be calculated and used to adjust the dataset. The data from multiple hybridisation experiments can then be stored in a suitable format, for example in an Access or SQL 7.0 database.

Comparison between arrays generates an output file containing the gene identifier and the fold-change in expression relative to the reference dataset. Fold change, (Tx vs Ty), is calculated by dividing the normalised spot density values of Tx with Ty. In this example, multiple time-course experiments are prepared and fold-change values calculated with reference to the T0 time point.

The fold change data derived from comparison of multiple hybridisation experiments can be analysed using a variety of approaches, including hierarchical clustering, (supervised or unsupervised), k-means clustering or self-organising maps. Software enabling these analyses includes the Cluster and Treeview software (M. Eisen, Stanford Uni, USA), J-Express European Bioinformatics Institute), GeneMaths (Applied Maths, Belgium) or GeneSpring (Silicon Genetics, USA). In this example hierarchical clustering is implemented using the GeneMaths software. Trees are generated using the WARD algorithm with distance calculated using the Pearson similarity metric. Attentively Euclidean distance metrics are used.

Simplification of Fold-Change Data

Following cluster analysis, fold-change data can be difficult to interpret owing to either a very large dataset and/or a wide range in fold change values. The visualization and interpretation of these datasets may be simplified using codes or combined codes. In this example, each unique gene is represented by at least two identical cDNAs on the array. The fold change value is calculated as described, then for each spot, a value above 5-fold change is accorded a code of 2, a fold-change value of less then 5 but greater then 2 is accorded a code of 1 and a fold-change value of less then 2 is accorded a code value of 0. A combined code is then derived by adding the code values for each identical cDNA on the array. The use of combined codes can greatly simplify the Cluster analysis and the subsequent visualisation (See FIG. 5).

Comparison of coordinate patterns of gene expression, by bioinformatic data analysis, using this model system, allows the identification of cell pathways and processes associated with apoptosis and survival.

In any given experiment or time course, ‘differentially regulated’ genes (combined code greater than or equal to 2) are identified and clustered by either normalised sDens (level of expression) or by fold chance values. Candidate genes, associated with apoptosis and survival, are those that are reproducibly differentially regulated in multiple experiments or time courses and are additionally ‘reciprocally regulated’ in conditions that permit apoptosis versus survival, respectively.

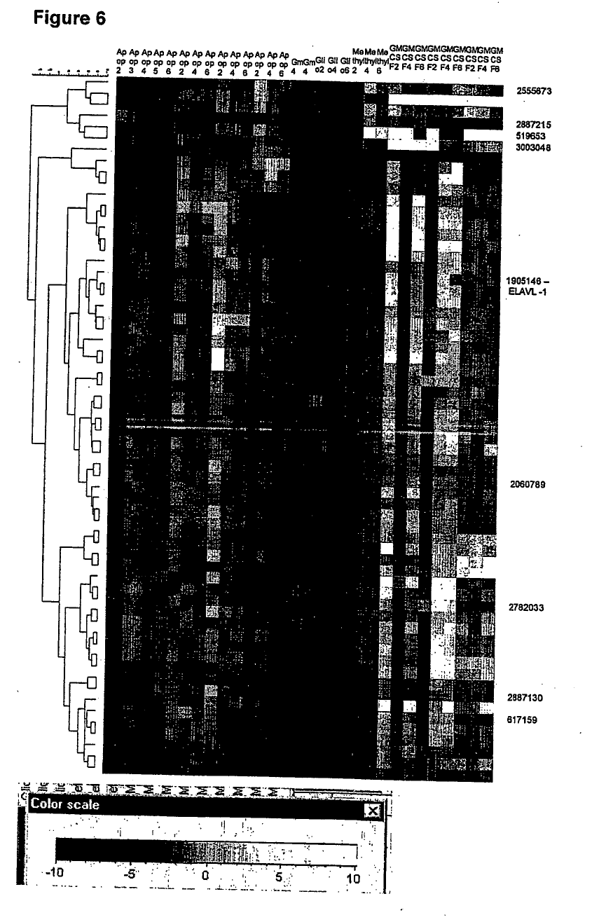

FIG. 6 shows the visual representation of a clustered selection of candidate neutrophil apoptosis/survival-associated genes identified of LifeGrid filters. Each row represents the differential regulation of an individual gene. The Fold Change colour scale is shown. Experiments measuring neutrophil apoptosis, GM-C SF inhibition of apoptosis and Gliotoxin blockage of GM-CSF inhibition of apoptosis were as follows:

Neutrophil Apoptosis

Four representative neutrophil apoptosis time course experiments are represented (Apop), with RNA samples isolated at 2 h (Apop2), 3 h (Apop3), 4 h (Apop4), 5 h (Apop5) and 6 h (Apop6) post-isolation of neutrophils. Fold change values are expressed relative to zero hour control samples.

Inhibition of Neutrophil Apoptosis by Treatment with GM-CSF

Three representative GM-CSF time course experiments are represented (GM-CSF), with RNA samples isolated at 2 h (GMCSF2), 4 h (GMCSF4) and 6 h (GMCSF6) post-treatment with GM-CSF. Fold change values are expressed relative to zero hour control samples.

Blockage of GM-CSF-Mediated Inhibition of Neutrophil Apoptosis by Treatment with Gliotoxin

Three representative Gliotoxin time course/experiments are represented. In one, GM-CSF is added in the presence of Gliotoxin (Glio) or an inactive analogue Methyl Gliotoxin (Methyl), with RNA samples isolated at 2 h (Glio2 and Methyl2), 4 h (Glio4 and Methyl4) and 6 h (Glio6 and Methyl6) post-treatment with GM-CSF. Fold change values are expressed relative to zero hour control samples. In the remaining two experiments (GM 4) RNA samples are isolated at 4 h post-treatment with GM-CSF, and fold change values are expressed relative to Methyl Gliotoxin control samples.

Each experimental RNA sample, profiled by microarray, represents the pool of multiple experiments carried out on neutrophils isolated from individual human donors. The number of donor samples used for each experiment/time course is summarised in Table 1.

| Experiment | Donors |
|---|---|
| Apop 2, 3, 4, 5, 6 | n = 7 |
| Apop 2, 4, 6 | n = 17 |
| Apop 2, 4, 6 | N = 8 |
| Apop 2, 4, 6 | n = 8 |
| GM4 | n = 7 |
| GM4 | n = 7 |
| Glio 2, 4, 6 | n = 3 |
| Methyl 2, 4, 6 | n = 3 |
| GMCSF 2, 4, 6 | n = 9 |
| GMCSF 2, 4, 6 | n = 12 |
| GMCSF 2, 4, 6 | n = 11 |

Average fold change values (from two spots on the array filters) are clustered with GeneMaths, using a Pearson correlation and Ward clustering algorithm.

Candidate genes represented in this selection share similar overall expression characteristics, that of an ‘apoptosis/survival cluster’. Candidate genes tend to be down-regulated (dark) in multiple experiments and time courses for apoptosis (Apop, GM and Glio; see legend) and up-regulated (light) in experiments and time courses for survival (Methyl and GMCSF; see legend).

One of the differentially expressed genes associated with apoptosis and survival is identified as ELAVL-1.

### EXAMPLE 2

### ELAV-Like 1 mRNA is Increased in GM-CSF-Induced Neutrophil Survival, and this Increased Expression is Blocked by Gliotoxin

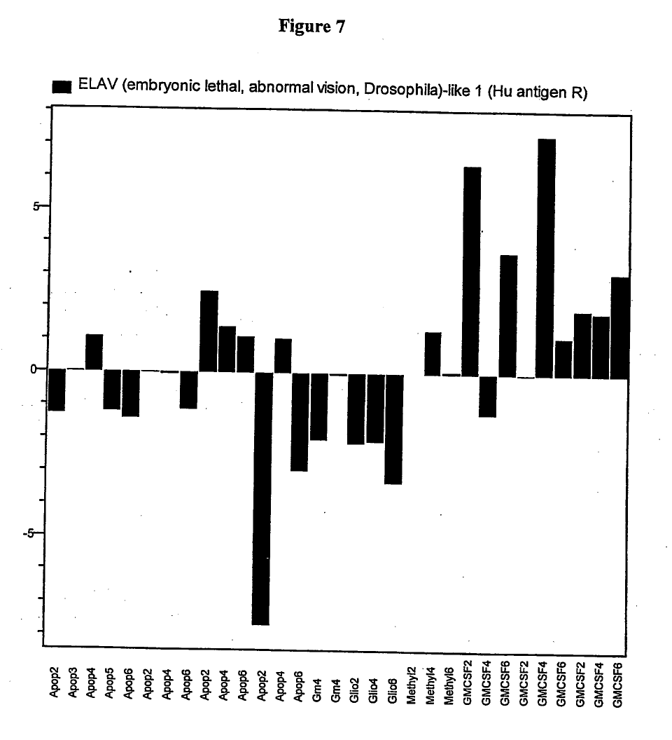

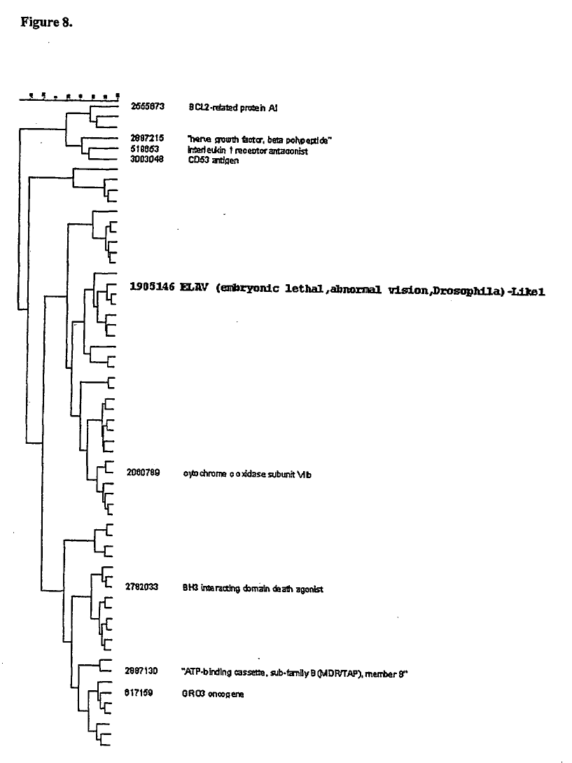

FIG. 7 shows the relative amounts of ELAVL-1 transcripts isolated from neutrophils treated according to Example 1. Experimental conditions and cluster analysis of average fold change comparisons are as described in Example 1.

Expression of ELAVL-1 is up-regulated in multiple experiments between 2 and 6 h following addition of GM-CSF. Up-regulated genes may represent potential survival factor genes, which block or delay the apoptosis in neutrophils. Increase expression of ELAVL-1, following GM-CSF treatment, is blocked by the fungal inhibitor gliotoxin (Glio and GM; see legend).

### EXAMPLE 3

### Cluster Analysis and Gene Function

In our co-pending applications WO 01/46469 and WO 02/04657, we have established that gene function can be predicted by correlation to known genes that have a similar pattern of gene expression across multiple experiments. The use of bioinformatics cluster analysis to identify novel pathways and gene function is also described, for example, by Zhao et al. PNAS 98(10): 5631-5636, (2001); Heyer L J et al. Genome Res. 9(11):1106-15, (1999); Iyer V R et al. Science 283(5398):83-7, (1999); and in Gene Expr 7(4-6):387400 (1999).

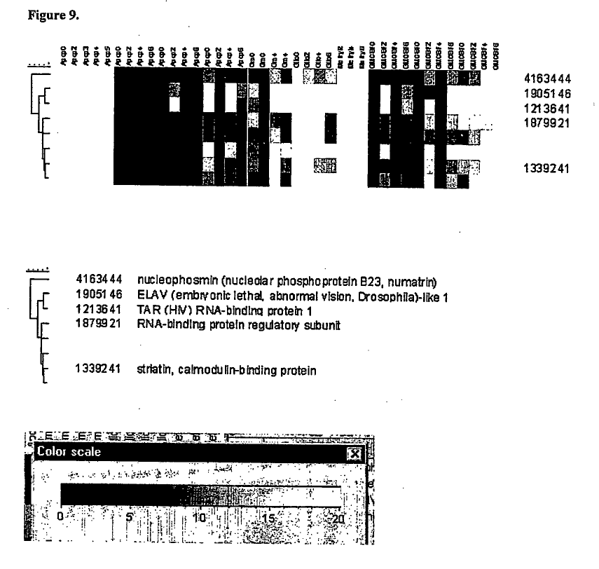

FIG. 8 shows a dendrogram representation of the association of candidate genes from the cluster analysis illustrated in FIG. 1 (performed using the method detailed in Example 1) of ELAVL-1 expression compared to other known genes that have a similar pattern of gene expression across multiple experiments. Amongst these are cytochrome c oxidase subunit VIIb (2060789), BH3 interacting domain death agonist (2782033), BCL2-related protein A1 (2555673), CD53 antigen (3003048), interleukin 1 receptor antagonist (519653), ATP-binding cassette, sub-family B (MDR/TAP), member (2887130), GRO3 oncogene (617159), stratifin (2028680) and nerve growth factor, beta polypeptide (2887215). All of these genes are known to be involved in apoptosis and survival. Several, including cytochrome c oxidase, CD53 and interleukin 1 receptor antagonist are also associated with Redox regulation.

Cytochrome c oxidase (COX), the terminal component of the respiratory chain complex of most aerobic organisms, is composed of 13 subunits in mammals. Mitochondrial release of cytochrome c is one of the principle stepps initiating the execution of apoptosis. Mitochondrial antisense RNA for cytochrome C oxidase can induce morphologic changes and cell death in human hematopoietic cell lines (Blood 1997 Dec. 1;90(11):4567-77). Apoptosis and ROS detoxification enzymes correlate with cytochrome c oxidase deficiency in mitochondrial encephalomyopathies (Mol Cell Neurosci 2000 Apr. ; 17(4):696-705).

BH3 interacting domain death agonist, otherwise known as-BID, is activated by the pro-apoptotic cascade. This causes BID to oligomerize BAK or BAX into pores that result in the release of cytochrome c. (for review see Cell Death Differ 2000 December ;7(12):1166-73).

BCL2-related protein Al, otherwise known as Bfl-1 was first isolated by Lin et al. (1993) as a novel mouse cDNA sequence, designated BCL2-related protein Al (Bcl2al) and was identified as a member of the Bcl-2 family of apoptosis regulators by the predicted protein sequence. An anti-apoptotic role of Bfl-1 is described in staurosporine-treated B-lymphoblastic cells (Int J Hematol 2000 December ;72(4):484-90).

CD53 is an N-glycosylated pan-leukocyte antigen of 35,000 to 42,000 MW. Increased expression of CD53 has been described on apoptotic human neutrophils (J Leukoc Biol 2000 Mar. ;67(3):369-73). Voehringer D W et al, described CD53 associated with resistance to ionising radiation, using microarray experiments. Expression of CD53 can lead to the increase of total cellular glutathione, which is the principle intracellular antioxidant and has been shown to inhibit many forms of apoptosis (Proc Natl Acad Sci USA 2000 Mar. 14;97(6):2680-5).

The Inter Leukin 1 receptor antagonist (IL1RN) is a protein that binds to IL1 receptors and inhibits the binding of IL1-alpha and IL1-beta. Overexpression of interleukin-1 receptor antagonist provides cardioprotection against ischemia-reperfusion injury associated with reduction in apoptosis (Circulation 2001 Sep. 18;104(12 Suppl 1):I308-I3). Hypoxia induces the expression and release of interleukin 1 receptor antagonist in mitogen-activated mononuclear cells (Cytokine 2001 Mar. 21;13(6):334-41). Overexpression of IL-1ra gene up-regulates interleukin-1beta converting enzyme (ICE) gene expression: possible mechanism underlying IL-1beta-resistance of cancer cells (Br J Cancer 1999 September ;81(2):277-86).

ATP-binding cassette, sub-family B (MDR/TAP) is homologous to MDR1 (multiple drug resistance). Increased expression and amplification of MDR1 sequences were also found in multidrug-resistant sublines of human leukemia and ovarian carcinoma cells. Overexpression of MDR1 appears to be a consistent feature of mammalian cells displaying resistance to multiple anticancer drugs and has been postulated to mediate resistance.

GRO3 oncogene: The GRO gene, a CXC chemokine otherwise known as macrophage inflammatory protein 1 beta (MIP-1B), was initially identified by Anisowicz et al. (1987) by its constitutive overexpression in spontaneously transformed Chinese hamster fibroblasts. Neutrophils have been shown regulate their own apoptosis via preservation of CXC receptors. Gro-alpha and IL-8 (CXC chemoldnes) suppress neutrophil apoptosis (Neu J Surg Res 2000 May 1;90(1):32-8).

Stratifin is one of the 14-3-3 family of proteins which mediate signal transduction by binding to phosphoserine-containing proteins. The 14-3-3 dimer binds tightly to single molecules containing tandem repeats of phosphoserine motifs, implicating bidentate association as a signaling mechanism with molecules such as Raf, Cbl and the pro-apoptotic molecule BAD (Cell 91: 961-971, 1997). Stratifin, is strongly induced by gamma irradiation and other DNA-damaging agents. The induction of 14-3-3-sigma is mediated by a p53-responsive element located 1.8 kb upstream of its transcription start site. Exogenous introduction of 14-3-3-sigma into cycling cells results in a G2 arrest (Molec. Cell 1: 3-11, 1997).

Nerve growth factor, beta polypeptide: Nerve growth factor is a well-characterised cytokine survival factor. NGF withdrawal induces apoptosis in a range of cells in-vitro and in-vivo. Nerve growth factor suppresses apoptosis of murine neutrophils (Biochem Biophys Res Commun 1992 Jul. 31;186(2):1050-6).

The close association of ELAVL-1 gene expression, across multiple reciprocal experiments, with a significant number of know apoptosis and survival genes identifies a function for ELAVL-1 in neutrophil and cellular apoptosis and survival.

### EXAMPLE 4

### ELAVL-1 is Adjacently Correlated with Two Other Known Regulators of RNA Stability

Translational regulation of gene expression is becoming an established feature of apoptosis, particularly following caspase activation (for review see Clemens et al; Cell Death Differ. 2000 7(7): 603-15).

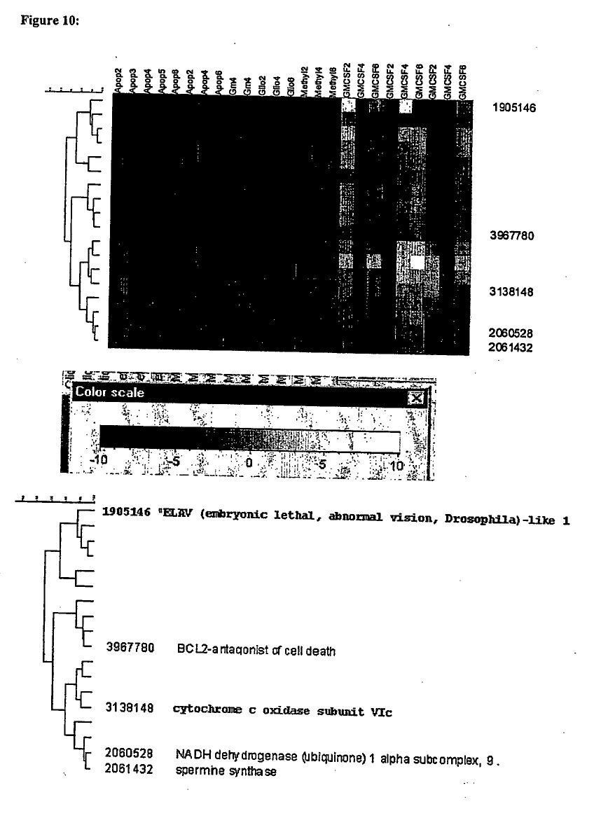

FIG. 9 shows a small sub-cluster (graphical representation and dendrogram) of eight genes with very closely related expression patterns across a range of reciprocal apoptosis and survival experiments. This cluster analysis was performed, for average normalised sDens (expression level) measurements, across >8,000 genes present on the Incyte LifeGrid filter, using Pearson correlation and Ward cluster algorithms. All experimental details are as for Example 1.

ELAVL-1 is adjacently correlated with four nuclear proteins, two of which are associated with regulation of RNA stability. One of these is associated with inhibiting differentiation and/or apoptosis in HL60 (a precursor to neutrophils), and another is associated with sensing oxidative stress.

Nucleophosmin: A nucleolar phosphoprotein that is more abundant in tumor cells than in normal resting cells. Stimulation of the growth of normal cells, e.g., mitogen activation of B lymphocytes, is accompanied by an increase in nucleophosmin protein level. Over-expression of nucleophosmin/23 decreases the susceptibility of human leukemia HL-60 cells to retinoic acid-induced differentiation and apoptosis, and also to sodium butyrate-induced apoptosis and inhibition of telomerase activity (Int J Cancer 2000 Nov. 1;88(3):392-400, Int J Cancer 1999 Dec. 10;83(6):765-71).

TAR (HIV) RNA-binding protein 1: HIV-1, the causative agent of acquired immunodeficiency syndrome (AIDS), contains an RNA genome that produces a chromosomally integrated DNA during the replicative cycle. The HIV Tat protein, a transcription-activating protein that binds to the bulge region of a stable stem-bulge-loop structure, TAR RNA, activates the HIV-1 long terminal repeat (LTR). TRBP1 activates the basal and the Tat-activated level of the HIV-1 LTR in human and murine cells (J Biomed Sci 2000 November-December ;7(6):494-506).

RNA-binding protein regulatory subunit, otherwise known as DJ-1. DJ-1 is suggested to be a novel mitogen-dependent oncogene product involved in a Ras-related signal transduction pathway. Recently, DJ-1 has been identified as a putative hydroperoxide-responsive proteins (HPRPs), and might be a sensitive indicator of oxidative stress status (Free Radic Res 2001 September ;35(3):301-10).

Striatin, otherwise known as SG2NA is a cell-cycle nuclear autoantigen containing WD40 motifs expressed mainly in S and G2 phase cells (Biochem Biophys Res Commun 1995 Feb. 27;207(3):1029-37). SG2NA has transcription activating property (Exp Cell Res 2001 Oct. 1;269(2):312-21) as well as an association with protein phosphatase 2A (J Biol Chem 2001 Jun. 29;276(26):24253-60).

Of the eight known marker genes that cluster among candidate genes (described in Example 1 and FIGS. 6 and 8) along with ELAVL-1, four have been previously associated as regulated, at least in part by translational regulation such as RNA stability. These include, GRO3 oncogene (Proc. Nat. Acad. Sci. 87: 7732-7736, 1990), interleukin-1 receptor antagonist (Cytokine 2001 Jun. 7;14(5):272-82), nerve growth factor (Brain Res Mol Brain Res 1998 Nov. 20;62(2):167-74) and cytochrome c oxidase (Biochem J 2000 Oct. 1;351(Pt 1):133-42). As well as interleukin-1 receptor antagonist, interleukin-1 itself and its receptor are co-ordinately regulated by RNA stability via the MAPK enzymes (J Biol Chem 2001 Feb. 2;276(5):3508-16). In addition, other genes that cluster among candidate genes (described in Example 1 and FIG. 6) along with ELAVL-1, such as Histimine releasing factor (otherwise known as Tumor protein, translationally controlled 1) are also known to be regulated by translational control (Biochem Int 1998 August ;19(2):277-86).

Taken together these results suggest that ELAVL-1 has a functional role in RNA stability and gene expression, associated with apoptosis and survival in human neutrophils.

### EXAMPLE 5

### ELAVL-1 Expression Correlates with Bcl-2 Expression

FIG. 10 shows a small sub-cluster (graphical representation and dendrogram) of eighteen genes with closely related expression patterns across a range of reciprocal apoptosis and survival experiments. This cluster analysis was performed, for average fold change measurements, across >8,000 genes present on the Incyte LifeGrid filter, using Pearson correlation and Ward cluster algorithms. All experimental details are as for Example 1.

ELAVL-1 expression compared to expression of a ‘known’ apoptosis and survival genes, including Bcl-2, cytochrome c oxidase subunit VIc, NADH dehydrogenase (ubiquinone) 1 alpha subcomplex, 9, and spermine synthase. These known genes have a similar pattern of gene expression across multiple experiments.

The multisubunit NADH:ubiquinone oxidoreductase (complex I) is the first enzyme complex in the electron transport chain of mitochondria. Antiapoptotic properties of Bcl-2 are related to the reduction of mitochondrial complex I activity (Biochem Biophys Res Commun 2001 Feb. 2;280(4):1021-7). The mitochondrial electron transport enzyme NADH:ubiquinone oxidoreductase (complex I), which is encoded by both mitochondrial DNA and nuclear DNA, is defective in multiple tissues in persons with Parkinson's disease (PD). Complex I may play an important role in the neurodegeneration of PD by fostering reactive oxygen species production and conferring increased neuronal susceptibility to mitochondrial toxins (Ann Neurol 1996 October ;40(4):663-71). NADH delydrogenase (ubiquinone) 1 alpha subcomplex 9 (NQO1) is one subunit in complex 1. Cells overexpressing NQO1 were resistant to dicoumarol, and this finding indicates the direct involvement of NQO1 in p53 stabilization. NQO1 inhibition induced p53 degradation and blocked wild-type p53-mediated apoptosis in gamma-irradiated normal thymocytes and in M1 myeloid leukemic cells that overexpress wild-type p53. Dicoumarol also reduced the level of p53 in its mutant form in M1 cells. The results indicate that NQO1 plays an important role in regulating p53 functions by inhibiting its degradation (Proc Natl Acad Sci USA 2001 Jan. 30; 98(3):1188-93).

Spermine synthase is 1 of 4 enzymes involved in the synthesis of polyamines from arginine and methionine. Some inducers of apoptosis, for example etoposide, absolutely require polyamines for caspase activation, yet the lack of polyamines, particularly spermine, strongly increases caspase activation when induced by UV irradiation (Biochem J 2001 Apr. 1;355(Pt 1):199-206). Spermine deficiency resulting from targeted disruption of the spermine synthase gene in embryonic stem cells leads to enhanced sensitivity to antiproliferative drugs (Mol Pharmacol 2001 February ;59(2):23 1-8).

Taken together these results suggest that ELAVL-1 has a functional role, associated with apoptosis and survival in human neutrophils.

### EXAMPLE 6

### ELAVL-1 Expression in Neutrophil Differentiation

ELAVL-1 is differentially regulated during neutrophil differentiation.

Nitroblue Tetrazolium (NBT) Reduction Test

HL60 cells are plated in 75 cm² flasks at a concentration of 0.5×10⁶/ml in RPMI+10% FCS (20 ml/flask) and incubated for the indicated period of time with 10 μM Retinoic acid after which time 1.5×10⁶ cells trypan blue negative cells are resuspended in 1 ml of RPMI medium and stimulated with 50 ng/ml Phorbal Myristate Acetate (PMA, Sigma) for 2 minutes. Nitroblue Tetrazolium salt is added to a final concentration of 50 μg/ml. Following incubation for 15 minutes at 37° C. the samples are placed on ice to terminate the reaction. Cells are then centrifuged at 300×g for 5 minutes and the supernatant removed. Cells are washed once in PBS and resuspended in 1 ml PBS. Cells are then cytocentrifiged onto glass slides using a cytospin (Shandon II). Slides are allowed to air dry and cells are then fixed in methanol (Rapi Diff Kit; Diachem Int, UK). A counter stain is applied by immersing the slides in an Eosin stain (Solution B; Rapi Diff Kit; Diachem Int, UK) for 10 minutes. Excess stain is removed by gentle washing with water. The slide is then air-dried and a cover slip applied Positive and negative cells are enumerated at 40× magnification Slides are assessed blind at 3 different fields and the mean calculated. NBT positive cells are determined as those that contained blue intracellular deposits. NBT negative cells are stained pink and are free of blue particles.

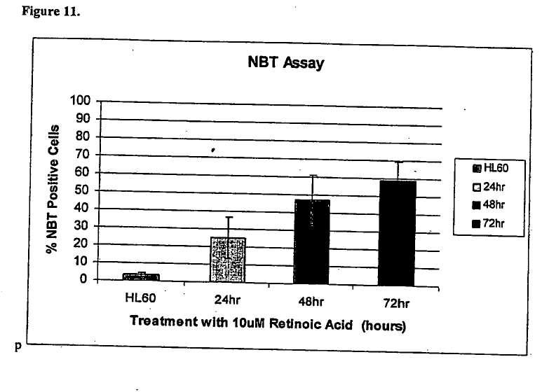

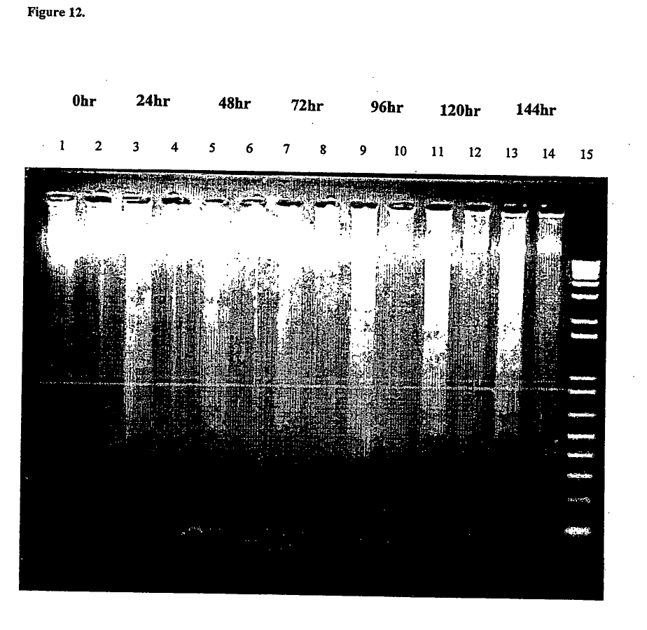

FIGS. 11 and 12 show time courses of neutrophil differentiation and apoptosis, respectively. Upon treatment of HL60 cells with retinoic acid, HL60 cells arrest their cell cycle and differentiate into neutrophils across a 5-day time course. Markers of differentiated neutrophils are increasingly detected at day 2 and day 3, as measured by reduction of NBT (see FIG. 11 and also Martin S J et al, Clin. Exp. Immunol. (1990)). Apoptosis begins to occur around day 4 (96 h) as shown in FIG. 12.

At the time points indicated RNA samples are isolated by lysing cells and adding RNAzol (5×10⁶ cells/ml RNAzol), purifying RNA as described previously and analysed by microarray using Incyte LifeGrid filters as described previously.

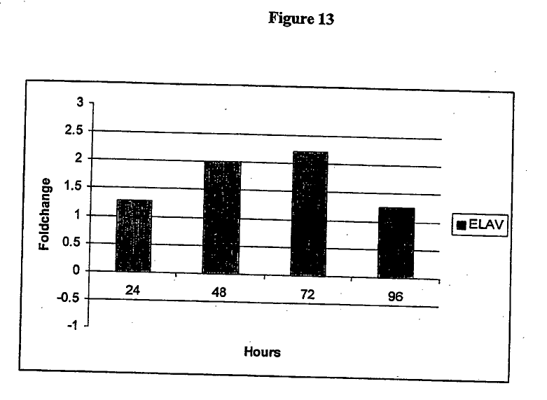

FIG. 13 shows ELAVL-1 gene expression fold change in retinoic acid treated HL60 cells.

Expression of ELAVL-1 increases throughout Day 1, 2 and 3 correlating with cell cycle arrest and neutrophil differentiation, and decreases on Day 4 correlating at the time when apoptosis is occurring.

These results demonstrate that ELAVL-1 expression is associated with the process of neutrophil differentiation and apoptosis following treatment with retinoic acid.

### EXAMPLE 7

### Effect of Cisplatin Treatment on ELAVL-1 Expression

HeLa cells are obtained from ATCC (Manassas, Va., USA), maintained in DMEM medium with 10% FCS at 37° C. in a 5% CO₂ atomosphere, and treated with cisplatin (1 μg/ml).

At the time points indicated RNA samples are isolated and analysed for gene expression changes by microarray using Incyte LifeGrid filters as described previously.

ELAVL-1 is decreased by cisplatin-induced apoptosis in HeLa cells.

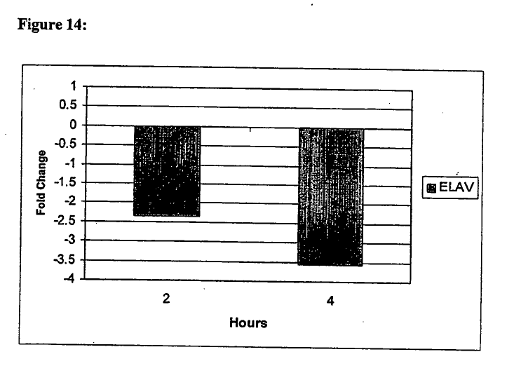

FIG. 14 shows ELAVL-1 gene expression fold change in Cisplatin treated HeLa cells.

### EXAMPLE 8

### Identification of a SNP

Single Nucleotide Polymorphisms, SNPs, are the most common genetic variations, occurring once every 100 to 300 bases. A key aspect of research in genetics is the association of sequence variation with heritable phenotypes. Informative SNPs accelerate the identification of disease genes by allowing researchers to look for associations between a disease and specific differences (SNPs) in a population. SNP distributions are also informative for drug response, and allow stratification of populations for particular therapy or drug treatment regimens.

The National Center for Bioinformatics (NCBI) SNP database (http://www.ncbi.nlm.nih.gov/SNP/index.html) was searched for informative SNPs using the gene name ELAVL1. A single SNP was identified from this database.

TABLE 2

| | | | | | | | | Amino |
|---|---|---|---|---|---|---|---|---|
| | Contig. | SNP rsft | Protein | | dbSNP | Protein | Codon | acid |
| | position | (cluster id) | accession | Function | allele | residue | position | position |
| NT 011145 | 397478 | rs14394 | XP 008947 | contig | t | Gly [G] | | |
| | | | | reference | | | | |
| | | | | Synonymous | c | Gly [G] | 3 | 268 |
| | | | | change | | | | |

Table 2 shows an informative SNP for the ELAV gene. A single SNP is identified mapping to amino acid 268. The SNP is a synonomous (silent) substitution, T-C transition, at the third position of the Gly-268 codon. The accession number of the chromosomal contig containing the ELAVL1 gene, and the position of the SNP within the contig are indicated. The unique SNP identification number is also included, as is the unique protein identifier

### EXAMPLE 9

### Identification and Analysis of ELAV-Like 1 Upstream Promoter Sequences

The genomic location of ELAV like 1 was achieved by alignment of known mRNA sequences onto assembled genomic DNA sequences using the DoubleTwist tools. An approximate 2 kb of DNA sequence upstream of the ELAV like 1 5′ UTR was identified, as described:

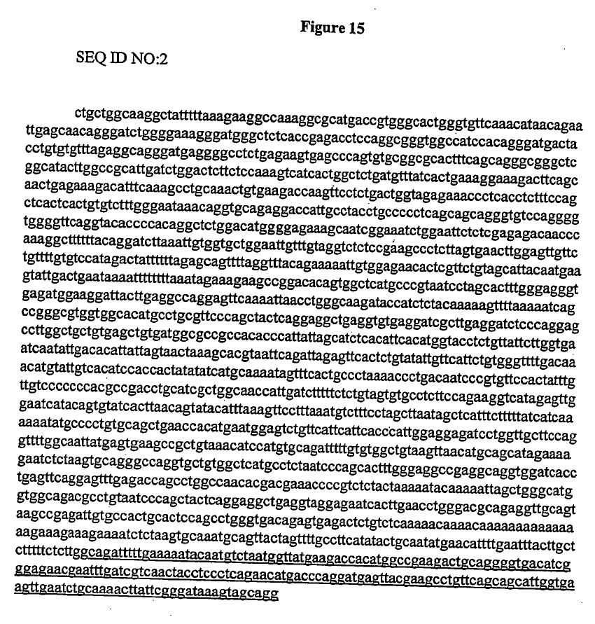

FIG. 15 (SEQ ID NO. 2) gives the sequence of the promoter region identified.

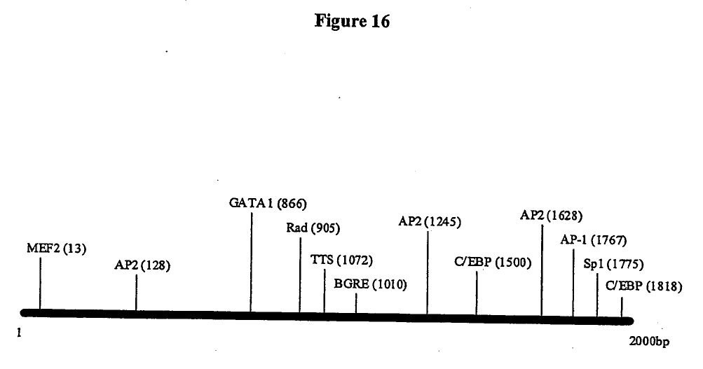

Analysis of this 2 kb upstream promoter sequence identified a number of characteristic promoter transcription factor and enhancer binding sites, as shown in FIG. 16. Numbers in brackets indicate positions derived from the sequence.

The following promoter sites are identified:

MEF2: Mammalian MEF-2 recognition site ([CT]TA[AT]AAATA[AG]) Faisst, S. & Meyer, S. Nucleic Acids Res 20: 3-26 (1992)

AP2: Mammalian AP-2 recognition site (G[CG][CG][AT]G[CG]CC) Mitchell, P. J. et al., Cell 50: 847-861 (1987)

GATA 1: Vertebrate GATA-1 recognition site ([AT]GATA[AC][CG][AGC]) Turpaev, K. T. & Vasetskii, E. S. Genetika 26: 804-816 (1990)

Rad: Yeast radiation damage control gene recognition site (CG[AT]GG[AT]*G[AC]A) Sebastian, J. et al., Mol Cell Biol 10: 4630-4637 (1990)

TTS: Possible eukaryotic transcription termination signal, ([Ac]GTGT[Ct][CA][AC]) McLauchlan, J. et al., Nucleic Acids Res. 13:1347-1368 (1985).

BGRE: Mammalian beta globin response element (CCACACCC) Myers, R. M. et al., Science 232: 613-618 (1986)

C/EBP: Mammalian C/EBP recognition site (T[GT]**G[CT]AA[GT]) Johnson, P. F. et al., Genes Dev 1: 133-146 (1987)

AP1: Mammalian AP-1 recognition site (TGA[CG]TAG) Turpaev, K. T. & Vasetskii, E. S. Genetika 26: 804-816 (1990) Sp1: Vertebrate Sp1 recognition site ([GT][AG]GGC[GT][AG][AG][GT]) Faisst, S. & Meyer, S. Nucleic Acids Res 20: 3-26 (1992)

### EXAMPLE 10

### Cloning and Functional Analysis of Upstream ELAV Like 1 Promoter Sequences

Primers are selected, using the Primer Designer facility of the GeneTool Lite software (Biotools Inc), which have minimal internal stability and annealing temperatures of 60° C.

Forward Primer:
5′ ttaaagaaggccaaaggcgcatga 3′
(position 19-42, Tm 60 degrees)
Reverse Primer:
5′ tcaaaaatctgccaagagaaaaagagcaag 3′
(position 1985-2014, Tm 60 degrees)

These primers amplify an amplimer size of 1996 bp.

PCR templates are prepared using genomic DNA purified from HL60 cells, isolated using the Qiagen ‘Blood and cell culture DNA mini Kit’, (Cat. 13323), as per manufacturers instructions. PCR amplifications are performed as described previously using 100 ng of genomic DNA as template. Amplimers are gel purified using the Qiaquick gel isolation kit (Qiagen, cat. 28706) and ligated to pCDNA3.1 using the Topo-TA cloning kit (Invitrogen, cat. 45-0005) according to the manufacturers instructions. Ligated DNA is transformed to E. coli (Top10). Transformants are selected for plasmid DNA preparation and sequence analysis.

Plasmid DNA is prepared using either the Qiagen miniprep (cat 27106) or midiprep (cat. 12643) kits as described by the manufacturer. Insert orientation is determined by PCR with ELAV-specific reverse primer and vector-specific forward primer (T7 primer). Plasmid miniprep DNA (100 ng to 5 μg) is sent to MWG Biotech or Lark Technologies for contract sequencing.

U937 cells are transfected with 10 μg of an EGFP reporter construct (pEGFP, Clontech), containing a 1996bp human genomic fragment driving the expression of the EGFP gene (Elav-EGFP). The fragment represents the region from −1986 to +10 of the transcription start site, including the putative ELAVL1 promoter region The 1996 bp fragment is cloned to the Topo-TA vector (described above) and subcloned to pEGFP using HindIII and XbaI restriction sites.

U937 macrophage cell line was cultured at 37° C. under 5% CO2 in RPMI supplemented with 10% foetal calf serum. Cells were transfected by the calcium phosphate method. Transfection of the pEGFP vector without the ELAV genomic fragment is used as a negative control whereas a construct containing the CMV promoter serves as a positive control.

U937 cells containing either pEGFP or ELAV-EGFP are treated with GM-CSF (50 Units) either in the presence or absence of gliotoxin (0.1 μg/ml). At the indicated periods of time, cells are examined for EGFP expression by flow cytometric analysis using a FacsCalibre (Becton Dickinson). Cells are considered positive for EGFP expression when the FL1 signal is greater than the background signal generated by either pEGFP or untreated ELAV-EGFP. All values are corrected for transfection efficiency by standardization against β-gal activity, derived from the cotransfected plasmid pSV β-gal (Promega).

An increase in fluorescence observed in U937 cells transfected with ELAV-EGFP when cultured in the presence of GM-CSF indicates that promoter activity is induced when cells are incubated in the presence of GM-CSF.

### EXAMPLE 11

### Full-Length Cloning of ELAV Like 1 coding mRNA Sequence

Primers are selected, using the Primer Designer facility of the DoubleTwist GeneTool Lite software (Biotools Inc), which have minimal internal stability and annealing temperatures of approximately 60° C. A HindIII restriction site (underlined) is incorporated to the 5′ end of the forward primer, to allow for orientation of the insert. Similarly a NotI restriction site (underlined) is incorporated to the 5′ end of the Reverse primer, to allow for orientation of the insert.

Forward Primer
5′-CCCAAGCTTACCCCCGCCCGCATCCAGATTTT-3′
Reverse Primer
5′AAAAGGAAAAGCGGCCGCTTCCGTACAAAAAAAAGCATGAGCG3′

PCR templates are prepared by reverse transcription of total RNA isolated from Hela or HL60 cell lines, or Brain and Kidney tissue samples (Stratagene). Briefly, total cellular RNA (1 μg to 20 μg) or polyA⁺ mRNA (100 ng to 5 mg) is incubated with an oligo (dT) primer. Primed RNA is reverse transcribed to first stand cDNA in a reaction containing M-MLV reverse transcriptase (RT; alternatively Superscript II is used (Life Sciences)), RT buffer, and dNTPs at 42° C. for 1 to 2 hours.

PCR reactions are prepared with primers (500 nMol), appropriate templates (1/100 dilution of the reverse transcription reaction), buffer and Taq polymerase (1 unit/reaction) (Qiagen) as directed by supplier. Reactions are subjected to 35 cycles of amplification with denaturation (94° C. 1 min), annealing (58° C. 1 min) and extension (72° C. 1 min). Products are analysed by gel electrophoresis.

Full-length amplimers are gel purified using the Qiaquick gel isolation kit (Qiagen, cat. 28706) and ligated to pCDNA3.1 using the Topo-TA cloning kit (Invitrogen, cat. 45-0005) according to the manufacturers instructions. Ligated DNA is transformed to E.coli (Top 10). Transformants are selected for plasmid DNA preparation and sequence analysis. Plasmid DNA is prepared using either the Qiagen miniprep (cat. 27106) or midiprep (cat. 12643) kits as described by the manufacturer. Insert orientation is determined by restriction digestion with HindIII.

Plasmid miniprep DNA (100 ng to 5 μg) is sent to MWG Biotech (UK) or Lark Technologies (UK) for contract sequencing. Sequencing reactions are primed using one of the following universal primer sequences:

M13 (-24) Reverse Primer:
5′ aac agc tat gac cat g 3′
M13 (-48) Reverse Primer:
5′ agc gga taa caa ttt cac aca gga 3′
M13 (-20) Forward Primer:
5′ gta aaa cga cgg cca gt 3′
M13 (-40) Forward Primer:
5′ gtt ttc cca gtc acg ac 3′
T3 Primer:
5′ aat taa ccc tca cta aag gg 3′
T7 Primer:
5′ gta ata cga ctc act ata ggg c 3′
BGH Primer:
5′tag aag gca cag tcg agg 3′

Identity of full-length clones is confirmed by Blast homology analysis.

### EXAMPLE 12

### Functional Analysis of ELAV Like 1 Expression in HeLa Proliferation ‘Plaque’ Assay

Typically, gene function associated with proliferation, survival and death (apoptosis) can be ascertained by the expression of the recombinant gene (mRNA) in a test (model) system by measurement of impact on cell growth and viability. We have established a model ‘plaque assay’ using HeLa cells to measure this effect (see also Cancer Research 2000 60(16) 4654-60). The ‘readout’ in this assay identifies a gene function as a ‘modulator of cell growth/survival’.

HeLa cells are plated into a 24 well tissue culture plate at a concentration of 1.5×10⁴/ml. Cells are left to adhere overnight, before transfecting the cells with a pcDNA3.1 plasmid containing the gene of interest (full-length coding mRNA sequence) using a Calcium Phosphate transfection kit (Clontech). Cells are left in the transfection medium for 24 h, prior to replacing it with fresh culture medium. Following 24 h, transfected cells are treated with G418 (an antibiotic to select for cells containing an integrated copy of the plasmid and gene of interest, by virtue of the plasmid containing and expressing a gene for neomycin resistance) at a concentration of 500 μg/ml and culture maintained for a further 7 days or until cells in test or control wells become confluent. Cells are then fixed and stained with Crystal Violet (1% in ethanol) for five minutes. To quantify, cells are solubilized by adding 33% Acetic Acid and the absorbance measured by reading the plate at 570 nm using a calorimetric plate reader.

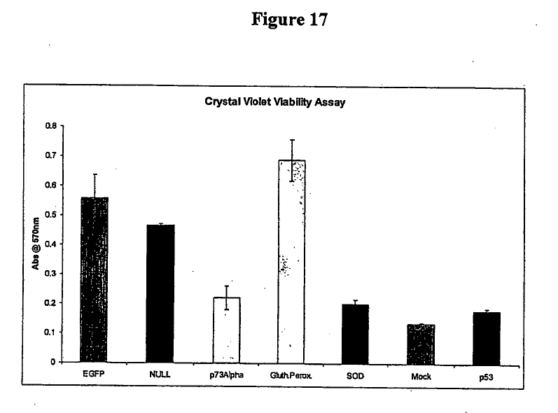

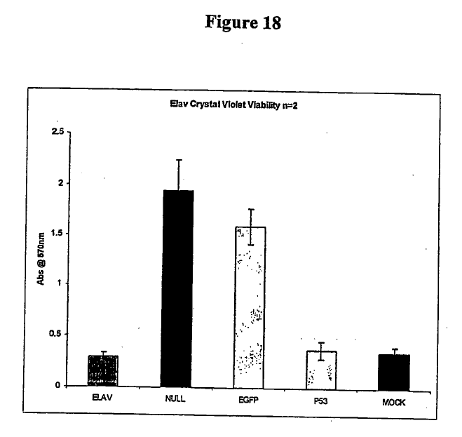

This assay is validated by a number of control genes, which are known to affect cell growth/survival, including superoxide dismutase (SOD), glutathione peroxidase, p53 and p73. Superoxide dismutase and glutathione peroxidase are known redox modulators and cell survival factors; SOD was also identified from our neutrophil model by cluster analysis, as in our copending applications WO 01/46469 and WO 02/04657. p73 and p53 are known tumor suppressor genes, which induce cell apoptosis. FIG. 17 shows graphical representation of the effect of these known survival and pro-apoptotic genes on the proliferation/viability of HeLa cells, as determined by a plaque assay. It should be stressed that in this assay, a significant increase or decrease in cell proliferation/viability is a functional identification of a ‘modulator of cell growth/survival’. For, instance both SOD and glutathione peroxidase are known redox modulators and survival factors but in this assay SOD decreases proliferation/viability in this assay while glutathione peroxidase increases proliferation/viability. Both p73 and p53 decrease proliferation/viability. These results, and those for p73 and p53, in HeLa cells are consistent with the reported data (Cell Growth Differ 1996 September ;7(9):1175-86; J Cell Physiol 1998 June ;175(3):359-69). This is a well-established ‘model’ system to validate gene function and so is not expected to replicate precisely the cellular context of these genes in the human primary neutrophil.

FIG. 18 shows graphical representation of the effect of ELAV like 1 on the proliferation/viability of HeLa cells, as determined by a plaque assay. Expression of recombinant ELAVL-1in HeLa cells resulted in significant inhibition of proliferation/viability, at an equivalent level to that elicited by the tumor suppressor gene p53. These results identify ELAV-like 1 as a ‘modulator of cell growth/survival’.

### EXAMPLE 13

### Expression of Recombinant ELAVL-1 Confers Survival in HeLa Cells to the Chemotherapeutic Drug Cisplatin

HeLa cell lines are as described in Example 12 with G418 selection maintained upto 48 hours prior to assay.

Cells stably expressing ELAVL-1 full-length cDNA in pcDNA3.1, or EGFP and mitochondrial superoxide dismutase (SOD) as a controls, are plated at 1-2×10³ cells/well of a 96 well plate and allowed to adhere overnight. Cells are then exposed to a range of cisplatin concentrations (1-10 μg/ml) for a further 24 h after which the cells are washed free of drug and cultured for 96 hours when cell survival is determined by MTT. For the last 4 hours, MTT (0.5 mg/ml) is added to the culture and the subsequent formazan crystals are released with DMSO. Results are mean of three separate wells.

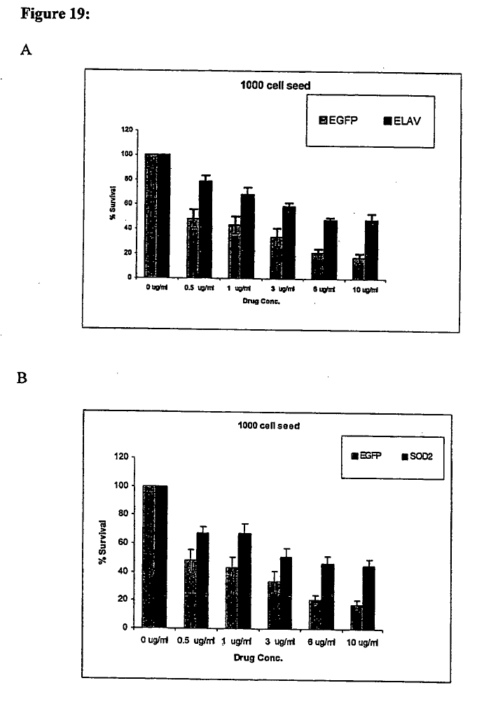

FIG. 19A shows that expression of ELAVL-1 confers resistance to cisplatin-induced cell death. At doses of cisplatin between 0.5 and 10 μg/ml percentage survival is significantly higher than control cells. Resistance to cisplatin-induced cell death is comparable to that with the expression of mitochondrial SOD (see FIG. 19B) a known survival gene.

### EXAMPLE 14

### Expression of Recombinant ELAVL-1 is Associated with Changes in Gene Expression, Similar to that of Expression of Recombinant p53 and SOD

This example describes the analysis of oligonucleotide/polynucleotide sequences whose expression changes are associated with expression of ELAV-like 1.

In this example, commercial microarrays are used to measure global gene expression, associated with ELAV-like 1 expression in HeLa cells. Analysis of such microarray results identifies genes whose expression pattern changes (either up-regulation or down-regulation) in a functional association with expression of recombinant ELAV-like 1. We demonstrate that the pattern of genes isolated using this approach are similar to those associated with expression of p53 and SOD, and include many genes whose products have been associated with apoptosis and survival. This identification further establishes a functional cellular role of ELAV-like 1 in the modulation of growth and survival.

HeLa cells are transiently transfected with pcDNA3.1 containing full-length cDNA for ELAVL-1, or Bcl-2, HB24, TTF, SOD and p53 using the CalPhos Mammalian Transfection Kit (Clontech) according to the manufacturers instructions. HeLa cells are plated in 75 cm² flasks at a concentration of 1.5×10⁶ cells/flask. The following day, when cells are 70% to 80% confluent cells are transfected and the Cal/Phos soln left on the cells for a further 18 hours at 37° C. in a 5% CO₂ humidified incubator. Typically, this transfection procedure yield 60-70% transfection efficiency, as judged by FacsCalibre analysis of EGFP transfected cells. Subsequently, the medium is replaced and cells are cultured for a further 24 hours before each sample is washed twice with PBS and lysed by addition of 1 ml of RNAzol.

To the 1 ml of RNAzol is added 100 μl of chloroform and the solution is centrifuged. The aquous layer is removed and the RNA is precipitated following addition of an equal volume of ice-cold isopropanol and centrifugation for 20 mins at 12000 g at 4° C. The RNA is further cleaned by addition to an RNeasy minispin column (Qiagen) according to the manufacturers instructions. Any contaminating DNA remaining in the elutant is removed by DNAase treatment of samples.

Measurement of global gene expression by ‘Microarray’ is carried out according to the method outlined in Example 1. cDNA is hybridised to Human Life Grid™ arrays and subjected to quantitative imaging and analysis using a STORM phosphoimager.

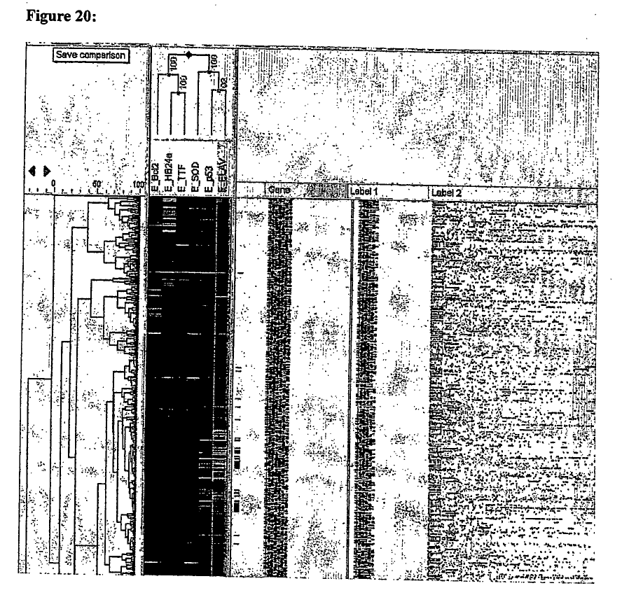

FIG. 20 shows the visual representation of a cluster analysis of comparative transient transfection of HeLa cells with full-length cDNAs for ELAVL-1, p53 and Bcl-2, HB24, TTF and SOD. Each data set represents the expression of >8,000 individual genes from Incyte LifeGrid array filters. For each experiment average fold change values are calculated relative to transient transfected ‘empty’ control expression vector. Average fold change values (from two spots on the array filters) are clustered with GeneMaths, using a Pearson correlation and Ward clustering algorithm. Cluster analysis is both between individual genes on the array and also between arrays. Therefore, similar overall expression patters across the entire array can be associated. The Fold Change colour scale is shown.

Transient transfection of HeLa cells with ELAVL-1 is associated with significant changes in gene expression. The overall pattern of gene expression changes are very similar to those induced by the ectopic expression of HeLa cells with p53, and these arrays are clustered together as compared to the other arrays in the experiment, as illustrated by the dendrogram.

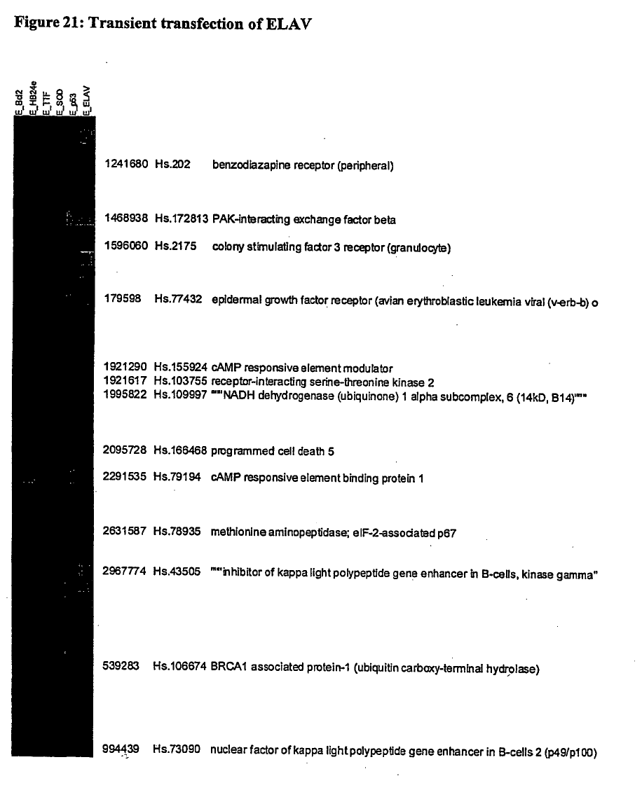

FIG. 21 shows a subset of the the visual representation of a cluster analysis of comparative transient transfection of HeLa cells with full-length cDNAs for ELAVL-1, p53 and Bcl-2, HB24, TTF and SOD, represented in FIG. 20. This subset represents those genes that are coordinately regulated by both p53 and ELAVL-1 as compared to other arrays. Average fold change values (from two spots on the array filters) are clustered with GeneMaths, using a Pearson correlation and Ward clustering algorithm. The Fold Change colour scale is shown.

Many of these gene changes are known to be associated with apoptosis and survival, as described, other are unknown and novel ESTs, which may represent potential therapeutic targets.

Benzodiazapine receptor (peripheral): Associated with the mitochondrial permeability transition pore, the benzodiazepine receptor is associated with apoptosis and survival (Biochem Pharmacol 2001 Mar. 15;61 (6): 695-705). Up-regulated by TNFalpha, expression is associated with increased cell survival (Biochem Pharmacol 2000 Dec. 1; 60(11): 163946)

PAK-interacting exchange factor beta: The WAF1-encoded protein p21 mediates p53 suppression of tumor cell growth. Overexpression of p21 in a tumor cell line suppresses colony formation similar to that resulting from p53 overexpression, p21-activated kinases, or PAKs bind to and are activated by Rho family GTPases, such as CDC42 and RAC. PAKs are implicated in the regulation of gene expression, cytoskeletal architecture, and apoptosis.

Colony stimulating factor 3 receptor, otherwise know as the GM-CSF receptor: GM-CSF is a known neutrophil survival factor, as described herein.

Epidermal growth factor receptor: Epidermal growth factor is a well described survival factor (Curr Opin Cell Biol 1999 April ; 11 (2): 184-9).

cAMP responsive element modulator (CREM): CREM is a master-switch regulating the balance between differentiation and apoptosis in male germ cells (Mol Reprod Dev 2000 June ; 56 (2 Suppl):228-9).

Receptor-interacting serine-threonine kinase 2, otherwise known as RIP kinase or RICK: signaling through the FAS death receptor plays a critical role in the homeostasis of the immune systems. Following ligand-induced oligomerization, the FAS receptor recruits caspase-8 to the receptor signaling complex through FADD, which leads to processing and release into the cytosol of caspase-8. Active caspase-8 induces a cascade of caspases and the rapid demise of the cell. RIP is a death domain-containing protein kinase that interacts with the death domain of FAS. RICK is implicated in regulation of apoptosis included by the FAS receptor pathway (J. Biol. Chem. 273: 12296-12300, 1998).

Programmed cell death 5, otherwise known as TFAR19: Isolated from TFI cells (a human premyeloid cell line established from a patient with erythroleukemia) undergoing apoptosis after the withdrawal of GM-CSF from the culture medium. The expression of TFAR19 is upregulated in tumor cells undergoing aapoptosis and enhances apoptosis triggered by growth factor or serum deprivation (Biochem. Biophys. Res. Commun. 254:203-210, 1999).

cAMP responsive element binding protein 1 (CREB): Cyclic AMP (cAMP) second messenger pathways provide a chief means by which cellular growth, differentiation, and function can be influenced by extracellular signals. Following hormonal stimulation of a neuroendocrine cell, for example, increased cAMP levels activate cAMP-dependent protein kinase A, which phosphorylates 1 or more DNA-binding proteins. These in turn stimulate transcription of an array of cAMP-responsive genes. All cAMP-responsive gene promoters have in common an 8-base enhancer known as the cAMP-response element (CRE). CREB-binding protein is a nuclear integrator of nuclear factor-kappaB and p53 signaling (J Biol Chem 1999 Jan. 22;274 (4): 1879-82). The neutrophil antiapoptotic gene mcl-1 is up regulated by the phosphatidylionositol 3-linase/Akt signaling pathway through a transcription factor complex containing CREB (Mol Cell Biol 1999 September ; 19 (9): 6195-206). NGF-dependent survival of sympathetic neurons is mediated by CREB (Science 1999 Dec. 17, ; 286 (5448):2358-61)

Methionine aminopeptidase; eIF-2-associated p67: p67 binds to eukaryotic initiation factor-2 (eIF-2). p67 protects eIF-2-alpha from phosphorylation by eIF-2 kinases and thus promotes protein synthesis in the cell. During heme or serum deprivation, p67 is degraded and protein synthesis is inhibited (J. Biol. Chem. 268:10796-10801, 1993). Lowering the level of eukaryotic initiation factor 2-associated protein, p67, from mammalian cells by antisence, induced apoptosis (Exp Cell Res 1999 Feb. 1;246(2): 376-83).

Inhibitor of kappa light polypeptide gene enhancer in B cells, kinase gamma, otherwise known as NEMO: NEMO is an essential component of the NF-kappaB inducing I-kappaB kinase (IKK) complex (J Biol Chem 2001 Nov. 23;276(47):43708-12). Disruption of the X-linked gene encoding NF-kappa B essential modulator (NEMO) produces male embryonic lethality, completely blocks NF-kappa B activation by proinflammatory cytokines, and interferes with the generation and/or persistence of lymphocytes (Mol Cell 2000 June ; 5(6): 981-92).

BRCAI associated protein-1, otherwise known as BAPI: BAPI is tumor suppressor gene that functions in the BRCAI growth control pathway. BAPI enhances BRACI mediated inhibition of breast cancer cell growth (Ann N Y Acad Sci 1999; 886: 191-4). Nuclear factor of kappa light polypeptide gene enhancer in B-cells 2 (p49/p100): Activation of the transcription factor NF-kappaB has been linked to apoptosis, with the factor playing either an anti-apoptotic or a pro-apoptotic role, depending on the type of cell in which it is expressed (Curr Biol 1997 Feb. 1;7(2):R94-6).

These results suggest that ELAVL1 share functional similarities with p53 in its association with apoptosis and survival gene transcription modification.

### EXAMPLE 15

### Expression of ELAVL-1 in HeLa Cells Confers Resistance to Hydrogen Peroxide-Induced Cell Death

Cells stably expressing ELAVL-1 full length cDNA in pcDNA3.1, or EGFP and mitochondrial superoxide dismutase as a control, are plated at 1-2×103 cells/well of a 96 well plate and allowed to adhere overnight Cells are then exposed to a range of Hydrogen Peroxide (0-1320 μM) for a further 24 h after which cells are washed free of drug and cultured for 96 h when cell survival is determined by MTT. For the last 4 hours, MTT 0.5 mg/ml) is added to the culture and the subsequent formazan crystals are releaed with DMSO. Results are mean of three separate well.

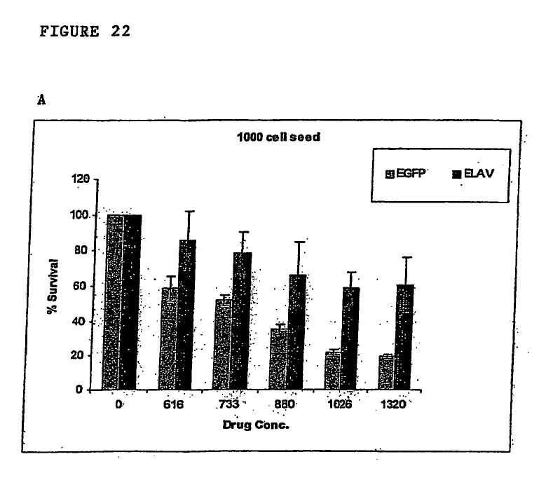

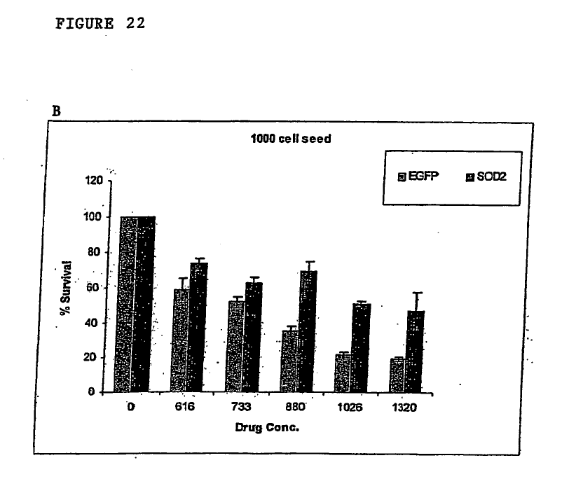

FIG. 22A shows that the expression of ELAVL-1 confers resistance to hydrogen peroxide -induced cell death. At all doses tested, the percentage survival is significantly higher than control cells. Resistance to hydrogen peroxide induced cell death is comparable to that with the expression of Mn SOD (see FIG. 22B) a known survival gene.

### EXAMPLE 16

### RNAi Knock-Down of ELAVL1 Expression Promotes Apoptosis of Growth-Factor Dependent Cell Line TF1

siRNA Oligos

The ELAVL1 CDS (1387 bp) is screened for AAN19TT siRNA target sequences with a GC content of 40-55%. Candidate targets are subject to a BLAST search The selected target (814-AATGTGAAAGTGATCCGCGAC-835) shares no significant homology with any other human genes. 21 nucleotide sense (UGUGAAAGUGAUCCGCGACTT) and antisense (GUCGCGGAUCACUUUCACATT) oligonucleotides are chemically synthesised as N19(RNA)+TT(DNA) by Eurogentec. The oligos are annealed at a concentration of 20 μM in annealing buffer (100 mM potassium acetate, 30 mM HEPES-KOH, pH 7.4, 2 mM magnesium acetate) for 1 min at 90° C. followed by 1 hour at 37° C. Duplex siRNAs are stored at −20° C. until required.

Hela Cells

The ELAVL1 siRNA is initially assayed for activity in Hela cells. Cells are plated at a density of 7.2×10⁴ in each well of a 6 well plate and incubated at 37° C. overnight in DMEM culture medium (1× Dulbeccos modified Eagles medium (Sigma D2554), 0.004% folic acid, 4 mM L-glutamine, 0.37% sodium bicarbonate, 0.1 mM sodium pyruvate) containing 10% foetal calf serum (FCS). 2 μg of duplex siRNA is transfected into Hela cells using DMRIE reagent (Invitrogen 10459-014) according to the manufacturer's recommendations. Additional wells of Hela cells are transfected with 2 μg of the unrelated siRNA against Lamin A/C (X03444). The latter sample serves as a positive control for RNAi and also demonstrates the specificity of the ELAVL1 knockdown by ELAVL1 siRNA.

The cells are harvested 24 hours post transfection by trypsinization and the levels of ELAVL1 transcript are compared by Q-PCR (quantitative polymerase chain reaction). RNA is isolated using Qiagen's RNeasy Miniprep columns (Qiagen 74104) after lysis on QIAshredder columns (Qiagen 79654). The RNA is quantified by spectrophotometric analysis and 1 μg is reverse transcribed into cDNA using SuperScriptII RNAseH-Reverse Transcriptase (Invitrogen 18064-014).

Q-PCR primers are designed to amplify a PCR product of 221 nucleotides in length from the CDS of ELAVL1, following the guidelines outlined in the Quantitect SYBR Green PCR handbook from Qiagen.

| | |
|---|---|
| Forward primer: | 720-CTCCTCCGGCTGGTGCATTTT; |
| Reverse primer: | 941-CTCCTCCGGCTGGTGCATTTT. |

Primers are synthesised by MWG Biotech

Two template standards are used for quantitative PCR assessment of mRNA levels:
1. A control RPS13 (NM_{—}001017) amplified PCR product diluted to [1000,100,10,1 and 0.1 fg] respectively, used to quantify the level of transcript;
2. RPS13 amplified from sample templates as separate PCR reactions for normalisation purposes.

The cDNA templates from HeLa cells transfected with either ELAVL1 or Lamin A/C siRNA, and a mock-transfected control, are amplified with ELAVL1 Q-PCR primers using QuantiTectSYBR Green PCR kit (Qiagen 204143) on a DNA Engine Opticon System (MJ Research). The amplification conditions include a 95° C. step for 15 min for initial activation of HotStarTaq DNA polymerase, followed by 35 cycles of (15 s at 94° C., 30 s at 60° C., 30 s at 72°). The fluorescence of the samples at 521 nm is read between the annealing and extension steps of the protocol. The melting curves are calculated at the end of the 35 cycles and confirm product homogeneity.

Results

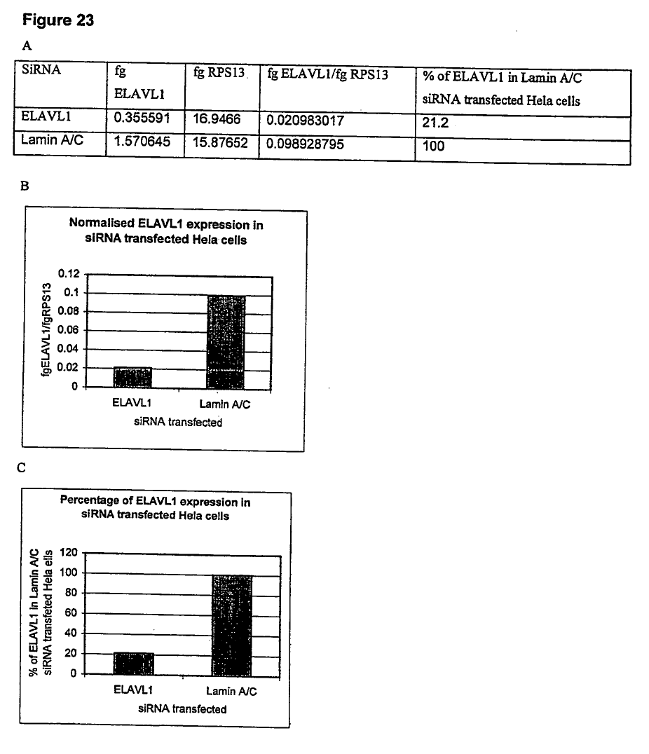

A standard curve is plotted using the log [template quantity] of the RPS13 control template dilutions (as above) versus cycle number at which the fluorescence intensity measured in the well exceeds the level specified in the cycle threshold parameters (the C(T) value). An estimate of the quantity of initial template in treatment samples is determined from this plot. Estimates are obtained for both ELAVL1 and RPS13 (FIG. 23A) (fg=femtogram). Subsequently, the amount of ELAVL1 present in each sample is normalized across samples by calculating the ratio of ELAVL1 to RPS 13 for each sample. The normalised expression values (shown in FIG. 23B) are used to estimate percentage knockdown of ELAVL1 with reference to the control transfected cells, (in this case cells transfected with Lamin A/C RNAi) (FIGS. 23A,C). The erythroleukaemic TF1 cell line is growth factor dependent, requiring GM-CSF for survival in culture. Withdrawal of GMCSF causes TF1 cells to undergo spontaneous apoptosis.

TF1 cells, 4×10⁵ cells/well, are transfected with 2 μg of siRNA in each of 7 wells of a 24 well plate using 8 μl DMRIE-C reagent (InVitrogen). Cells are simultaneously transfected in parallel with a control siRNA directed to Lamin A/C. Recombinant GMCSF is added to the OPTI-MEM media during transfection. The transfection media is replaced after four hours with fresh RPMI 1640 containing GMCSF and 10% FCS and incubated at 37° C. Twenty-four hours post-transfection, the media is changed; RPMI 1640+10% FCS containing GMSCF is added to 3 wells and RPMI 1640+10% FCS without GMSCF is added to the other 3 wells. These cells are harvested 48 and 72 hours post transfection for Q-PCR and phenotypic assays. Control TF1 cells, which are not transfected, are similarly treated. Samples are labelled T48+G, T48−G, T72+G, T72−G, according to the time of harvesting post-transfection and the presence (+G) or absence (−G) of GM-CSF in the media. RNA is isolated, cDNA is reverse transcribed and Q-PCR is performed as described above.

Survival assays used are:
1. Cell count,
2. Percentage of cells with Forward Scatter/Side Scatter (FSC/SSC) profiles of healthy cells and
3. Percentage of cells with in the Sub G1 phase of growth as a measure of the apoptosis in the cells.

1. Cell counts are determined using a haemocytometer. Cell viability is determined using the MTT assay. Briefly, 5 mg/ml MTT in PBS is added to 100 μl aliquots of cells, mixed thoroughly and incubated for 4 hours at 37° C. Mitochondrial succinate dehydrogenase of viable cells can convert the soluble MTT salt to an insoluble blue formazan crystal. Addition of 100 μl of 0.1N HCl/Isopropanol allows the samples to be read at 570 nm on a Molecular Devices Emax precision micro plate reader.

2. Analysis of TF-1 Apoptosis by Light Scatter Analysis Light Scatter Analysis takes advantage of the fact that by using the laser beam of a flow cytometer one can determine the size (Forward scatter) and granularity (Side Scatter) of a cell. The morphological changes associated with apoptosis, such as decreased size (shrinkage) and granularity affect these parameters. As a consequence, cells undergoing apoptosis will move to the left and slightly down, from the parameters of a healthy population

TF-1 cells are plated into. 24 well plates (2×10⁵/ml) and are cultured for 48 h in the presence or absence of GM-CSF (2 ng/ml). Cells are then harvested by centrifugation (1000 rpm, for 10 min) and washed in PBS. The pellet is resuspended in PBS (2×10⁵ cells/ml) and acquired by a FacsCalibre (HP Biosciences). Forward and Side scatter parameters are assessed using Cell Quest software.

3. SubG1 parameters are obtained by resuspending cells in a buffer, (0.1% Sodium Citrate, 0.1% TritonX-100, 200 μl of Propidium Iodide at Smg/ml made up to 20 mls in PBS), that permeabilizes the nuclei and allows fragmented DNA, degraded by endonucleases, to exit. As a result, cells that have undergone apoptosis contain less DNA and stain less intensely when stained with propidium iodide. Consequently, cells with fractional DNA content are located to the left of the G1 peaks (sub G1) on DNA frequency histograms. Cells are stained for 7 hours at 4° C. in the dark. The PI stained cells are then acquired by the flow cytometer. Analysis of FL2 fluorescence is performed on Cell Quest software to allow quantification of the Sub-G1 phase of the cell.

Results

Q-PCR

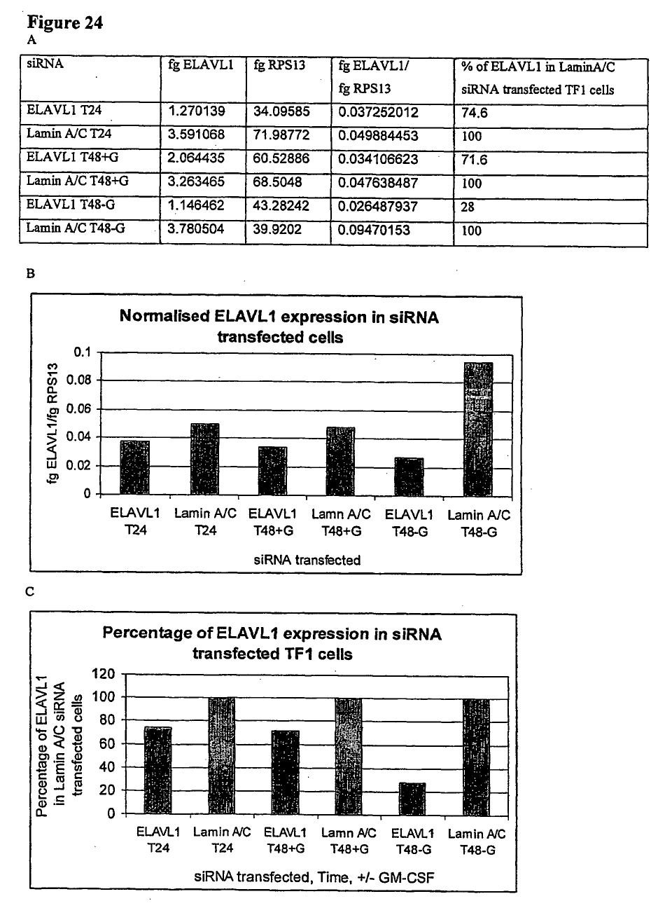

Transfection of ELAVL1 siRNA to TF1 cells reduces the expression of ELAVL1 mRNA (FIG. 24). There is a 25% reduction of ELAVL1 expression 24 hours after introduction of the RNAi, which is further decreased following prolonged incubation, (FIG. 24).

Survival

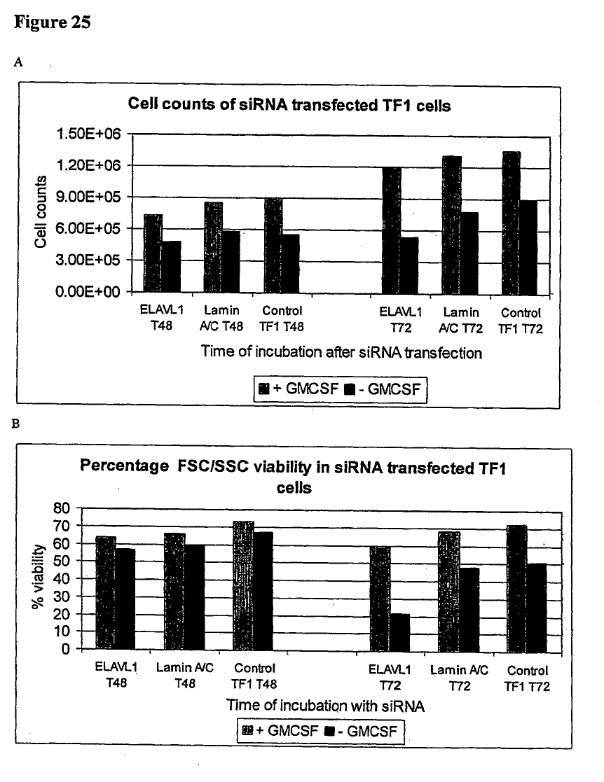

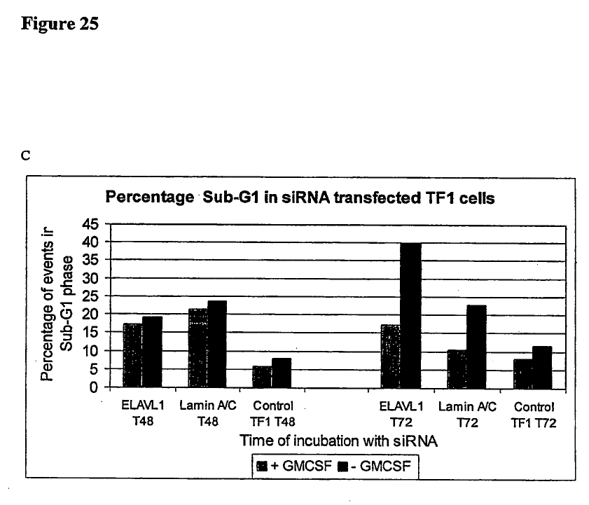

The survival status of these transfected cells is assayed at 48 and 72 hours post-transfection (representing 24 and 48 hours post ±GM-CSF). FIG. 25 shows the results of ELAVL1 siRNA on the viability of TF1 cells as measured by A. cell counts, B. FSC/SSC profile and C. Percentage Sub-G1.

In the presence of GMCSF, the ELAVL1 siRNA decreases overall cell count after 72 hours by 12%, compared to the control (non-transfected) cells. In the absence of GM-CSF, the ELAVL1 siRNA decreases overall cell count after 72 hours by 40% compared to control cells.

This reduction in viability due to ELAVL1 RNAi is reflected in the analysis of FSC/SSC profiles of the cell populations, which show 17% (+GM-CSF) and 58% (−GM-CSF) less cells falling within the live gate defined using control cells. Analysis of the Sub-G1 populations show that there is an increase in apoptosis, relative to the control cells, following transfection with the ELAVL1 siRNA.

In the presence of GM-CSF, the subG1 population increases from 8% (control) to 17% (treatment), assessed at 72 hours.

In the absence of GM-CSF, the subG1 population increases from 11% (control) to 40% (treatment), assessed at 72 hours.

These data suggest that inhibiting ELAVL1 expression, through specific down-regulation of mRNA levels, significantly reduces cell viability and increases apoptosis of the TF1 cells, both in the presence and absence of GM-CSF and thus demonstrate a key role for ELAVL1 in the regulation of survival and apoptosis.

### EXAMPLE 17 RNAi Knock Down of ELAVL1 Expression Promotes Apoptosis of HeLa Cells.

Oligofectamine Transfection of the Hela Cell Line

Hela cells are plated at 8.5×10⁴ cells per well of a 6 well plate in DMEM containing 10% FBS. Cells are allowed to adhere overnight @ 37° C. in a humidified atmosphere containing 5% CO₂ prior to transfection. Subsequently Hela cells are transfected with 2 ug of siRNA (as described in Example 16) using Oligofectamine reagent (In Vitrogen), according to the manufacturer's instructions for transfection of adherent cell lines. Transfection occurs overnight. The following day, transfection solution is removed from the monolayer and fresh DMEM/10% FBS is added. Analysis of Hela Cells is performed at the indicated time periods post transfection. Analyses include MTT, Sub G1 and FSC/SSC quantification of cell viability as described for the TF1 cell line.

Results

siRIA Targeting of ELAVL1 Increases the Amount of Death in HeLa Cell.

Analysis of HeLa cells at both 48 h and 72 h post transfection with the ELAVL1 siRNA was compared to a scrambled control. The scrambled control siRNA duplex is derived from the ELAVL1 siRNA by altering 4 bases of the sequence such that the siRNA can no longer function to downregulate ELAV mRNA levels, and similarly cannot activate RNA interference for any other known gene (as determined by BLAST sequence searches of the non-redundant databases). The sequence of the scrambled siRNA oligonucleotides is

| | |
|---|---|
| Sense: | UGAGAAUGUGAUGCGCGUCTT |
| antisense: | GACGCGCAUCACAUUCUCATT |

The altered nucleotides are underlined.

ELAVL siRNA increases the level of death among HeLa cells in culture. Cell death is analysed using three measurements, MTT, FSc/Ssc, and Sub G1 analysis as described previously.

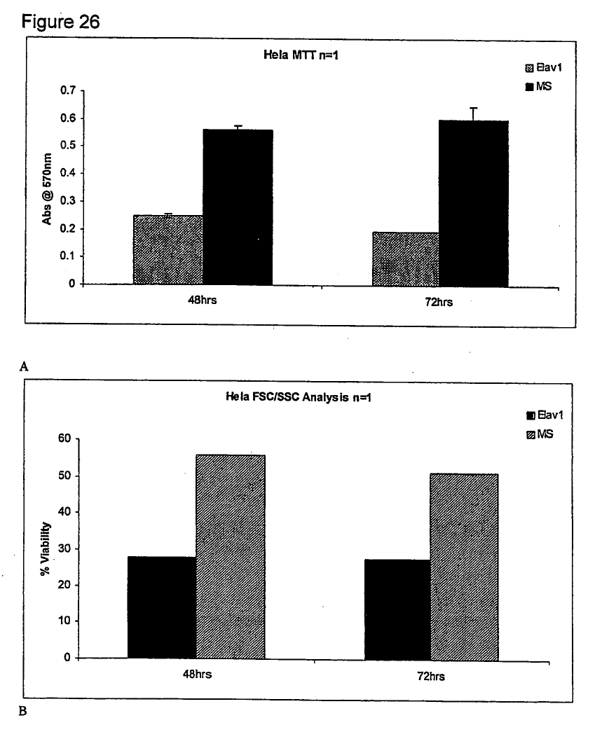

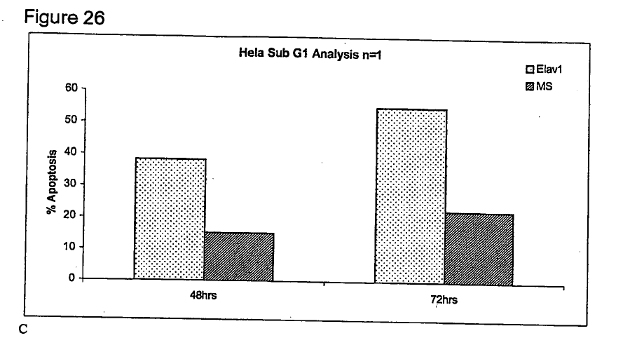

FIG. 26A shows MTT results of the effect of siRNA targeting Elav in HeLa cells relative to a scrambled control. A decrease in MTT absorption relative to scrambled control is indicative of a decrease in cell viability.

FIG. 26B shows Forward/Side Scatter results of the effect of siRNA targeting Elav in HeLa cells relative to a scrambled control. A decrease in the number of cells in the defined live gate relative to scrambled control is indicative of a decrease cell viability.

FIG. 26C shows Sub G1 analysis of the effect of siRNA targeting Elav in HeLa cells relative to a scrambled control. An increase in the number of cells with a Sub G1 profile relative to scrambled control is indicative of a decrease in cell viability.

All publications mentioned in the above specification, and references cited in said publications, are herein incorporated by reference. Various modifications and variations of the described methods and system of the present invention will be apparent to those skilled in the art without departing from the scope and spirit of the present invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A method for detecting apoptosis in a cell comprising detecting a decrease in any one of:
i) an ELAVL-1 polypeptide having an amino acid sequence as set out in SEQ ID NO: 1;
ii) a polypeptide having at least 80% homology with i);

2. A method of modulating apoptosis in a cell comprising the step of increasing, decreasing or otherwise altering the functional activity of
i) an ELAVL-1 polypeptide having an amino acid sequence as set out in SEQ ID NO: 1;
ii) a polypeptide having at least 80% homology with i);

3. A method as claimed in claim 2 comprising decreasing ELAVL-1 gene expression.

4. A method as claimed in claim 3 wherein ELAVL-1 gene expression is decreased by RNAi or by antisense treatment.

5. A method as claimed in claim 2 comprising increasing ELAVL-1 gene expression.

6. A method as claimed in claim 5 comprising:
a) providing an expression vector comprising a nucleic acid sequence encoding an ELAVL-1 polypeptide, said nucleic acid sequence being selected from the group consisting of:
i) a nucleic acid encoding an ELAVL-1 polypeptide having an amino acid sequence as set out in SEQ ID NO: 1;
ii) a nucleic acid which hybridises under stringent conditions to the sequence set out in i); or
iii) the complement of ii);
b) introducing the expression vector into the cell and maintaining the cell under conditions permitting expression of the encoded polypeptide in the cell.

7. A method for identifying a gene product whose expression is modulated by the expression of ELAVL-1 comprising the steps of:
i) providing a vector encoding ELAVL-1 as defined in claim 6;
ii) introducing said vector in a cell under conditions to promote expression of ELAVL-1; and
iii) measuring global gene expression associated with ELAVL-1 expression.

8. A method as claimed in claim 7 wherein global gene expression is measured by assaying gene transcription using a microarray.

9. A composition comprising a modulator of ELAVL-1 gene expression for use as a medicament.

10. A composition as claimed in claim 9 wherein said modulator of ELAVL-1 gene expression is selected from an antisense ELAVL-1 molecule and an RNAi ELAVL-1 molecule.

11. A method of treatment of a disease comprising administering a modulator of ELAVL-1 gene expression or functional activity to an individual.

12. A method as claimed in claim 11 wherein the modulator of ELAVL-1 gene expression is selected from an antisense ELAVL-1 molecule and an RNAi ELAVL-1 molecule.

13. A method as claimed in claim 11 or claim 12 wherein the disease is selected from cancer, an inflammatory disease, an autoimmune disease and a neurodegenerative disease.

14. An isolated nucleic acid molecule comprising a promoter, said nucleic acid sequence being selected from the group consisting of:
i) a nucleic acid molecule having the sequence set out in SEQ ID NO: 2;
ii) a nucleic acid molecule having at least 60% homology with i);
iii) a nucleic acid molecule hybridising under stringent conditions to i) or ii); and
iv) the complement of the sequences set out in i) to iii).

15. An isolated nucleic acid molecule as claimed in claim 14 which comprises at least one enhancer or transcription factor binding element selected from the group consisting of MEF 2, AP2, GATA 1, Rad, TTS, BGRE, C/EBP, AP1 and Sp1.

16. An isolated nucleic acid molecule as claimed in claim 14 or claim 15 which comprises a promoter sequence which is activated by GM-CSF.

17. A vector comprising a nucleic acid molecule as claimed in claim 14.

18. A vector as claimed in claim 17 wherein said nucleic acid molecule is operably linked to a reporter gene.

19. A method of identifying a compound that activates expression from the ELAVL-1 promoter comprising
i) transfecting a cell with a nucleic acid construct as claimed in claim 17 operably linked to a reporter gene;
ii) introducing a compound of interest;
iii) detecting ELAVL-1 gene expression by detecting the reporter gene product; and
iv) comparing with ELAVL-1 gene expression in the absence of the compound of interest.

20. An isolated nucleic acid molecule encoding ELAVL-1 and having a single nucleotide polymorphism at amino acid 268.

21. A method for identifying an agent that is capable of modulating apoptosis by modulating ELAVL-1 protein function comprising measuring RNA stability in the absence or presence of such agent.

22. A method as claimed in claim 21 comprising
i) taking ELAVL-1
ii) introducing a test compound
iii) incubating in the presence of mRNA
iv) measuring RNA stability

23. A method for identifying a compound that modulates apoptosis by modulating ELAVL-1 protein function comprising:
i) taking a cell expressing ELAVL-1;
ii) transfecting said cell with a nucleic acid construct encoding an ELAVL-1-regulated gene;
iii) introducing a test compound; and
iv) detecting expression of the ELAVL-1-regulated gene compared to expression in the absence of the test compound as an indication of modulation of ELAVL-1 protein function.

24. A method for modulating apoptosis in a cell comprising modulating a binding interaction between ELAVL-1 and an ARE-containing mRNA.


SEQUENCE LISTING (20 sequences)

SEQ ID NO: 1 - protein, 326 aa, Homo sapiens
MSNGYEDHMAEDCRGDIGRTNLIVNYLPQNMTQDELRSLFSSIGEVESAKLIRDKVAGHSLGYGFVNYVTAKDAERAINTLNGLRLQSKTIKVSYARPSSEVIKDANLYISGLPRTMTQKDVEDMFSRFGRIINSRVLVDQTTGLSRGVAFIRFDKRSEAEEAITSFNGHKPPGSSEPIAVKFAANPNQNKNVALLSQLYHSPARRFGGPVHHQAQRFRFSPMGVDHMSGLSGVNVPGNASSGWCIFIYNLGQDADEGILWQMFGPFGAVTNVKVIRDFNTNKCKGFGFVTMTNYEEAAMAIASLNGYRLGDKILQVSFKTNKSHK

SEQ ID NO: 2 - DNA, 2195 bp, Homo sapiens
ctgctggcaaggctatttttaaagaaggccaaaggcgcatgaccgtgggcactgggtgttcaaacataacagaattgagcaacagggatctggggaaagggatgggctctcaccgagacctccaggcgggtggccatccacagggatgactacctgtgtgtttagaggcagggatgaggggcctctgagaagtgagcccagtgtgcggcgcactttcagcagggcgggctcggcatacttggccgcattgatctggactcttctccaaagtcatcactggctctgatgtttatcactgaaaggaaagacttcagcaactgagaaagacatttcaaagcctgcaaactgtgaagaccaagttcctctgactggtagagaaaccctcacctctttccagctcactcactgtgtctttgggaataaacaggtgcagaggaccattgcctacctgccccctcagcagcagggtgtccaggggtggggttcaggtacaccccacaggctctggacatggggagaaagcaatcggaaatctggaattctctcgagagacaacccaaaggcttttttacaggatcttaaattgtggtgctggaattgtttgtaggtctctccgaagccctcttagtgaacttggagttgttctgttttgtgtccatagactattttttagagcagttttaggtttacagaaaaattgtggagaacactcgttctgtagcattacaatgaagtattgactgaataaaattttttttaaatagaaagaagccggacacagtggctcatgcccgtaatcctagcactttgggagggtgagatggaaggattacttgaggccaggagttcaaaattaacctgggcaagataccatctctacaaaaagttttaaaaatcagccgggcgtggtggcacatgcctgcgttcccagctactcaggaggctgaggtgtgaggatcgcttgaggatctcccaggagccttggctgctgtgagctgtgatggcgccgccacacccattattagcatctcacattcacatggtacctctgttattcttggtgaatcaatattgacacattattagtaactaaagcacgtaattcagattagagttcactctgtatattgttcattctgtgggttttgacaaacatgtattgtcacatccaccactatatatcatgcaaaatagtttcactgccctaaaaccctgacaatcccgtgttccactatttgttgtcccccccacgccgacctgcatcgctggcaaccattgatctttttctctgtagtgtgcctcttccagaaggtcatagagttggaatcatacagtgtatcacttaacagtatacatttaaagttcctttaaatgtctttcctagcttaatagctcatttctttttatcatcaaaaaatatgcccctgtgcagctgaaccacatgaatggagtctgttcattcattcacccattggaggagatcctggttgcttccaggttttggcaattatgagtgaagccgctgtaaacatccatgtgcagatttttgtgtggctgtaagttaacatgcagcatagaaaagaatctctaagtgcagggccaggtgctgtggctcatgcctctaatcccagcactttgggaggccgaggcaggtggatcacctgagttcaggagtttgagaccagcctggccaacacgacgaaaccccgtctctactaaaaatacaaaaattagctgggcatggtggcagacgcctgtaatcccagctactcaggaggctgaggtaggagaatcacttgaacctgggacgcagaggttgcagtaagccgagattgtgccactgcactccagcctgggtgacagagtgagactctgtctcaaaaacaaaacaaaaaaaaaaaaaaagaaagaaagaaaatctctaagtgcaaatgcagttactagttttgccttcatatactgcaatatgaacattttgaatttacttgctctttttctcttggcagatttttgaaaaatacaatgtctaatggttatgaagaccacatggccgaagactgcaggggtgacatcgggagaacgaatttgatcgtcaactacctccctcagaacatgacccaggatgagttacgaagcctgttcagcagcattggtgaagttgaatctgcaaaacttattcgggataaagtagcagg

SEQ ID NO: 3 - DNA, 10 bp, Artificial sequence
  Mammalian MEF-2 recognition site
ytawaaatar

SEQ ID NO: 4 - DNA, 24 bp, Artificial sequence
  Primer
ttaaagaaggccaaaggcgcatga

SEQ ID NO: 5 - DNA, 30 bp, Artificial sequence
  Primer
tcaaaaatctgccaagagaaaaagagcaag

SEQ ID NO: 6 - DNA, 32 bp, Artificial sequence
  Primer
cccaagcttacccccgcccgcatccagatttt

SEQ ID NO: 7 - DNA, 43 bp, Artificial sequence
  Primer
aaaaggaaaagcggccgcttccgtacaaaaaaaagcatgagcg

SEQ ID NO: 8 - DNA, 16 bp, Artificial sequence
  Primer
aacagctatgaccatg

SEQ ID NO: 9 - DNA, 24 bp, Artificial sequence
  Primer
agcggataacaatttcacacagga

SEQ ID NO: 10 - DNA, 17 bp, Artificial sequence
  Primer
gtaaaacgacggccagt

SEQ ID NO: 11 - DNA, 17 bp, Artificial sequence
  Primer
gttttcccagtcacgac

SEQ ID NO: 12 - DNA, 20 bp, Artificial sequence
  Primer
aattaaccctcactaaaggg

SEQ ID NO: 13 - DNA, 22 bp, Artificial sequence
  Primer
gtaatacgactcactatagggc

SEQ ID NO: 14 - DNA, 18 bp, Artificial sequence
  Primer
tagaaggcacagtcgagg

SEQ ID NO: 15 - DNA, 21 bp, Homo sapiens
aatgtgaaagtgatccgcgac

SEQ ID NO: 16 - DNA, 21 bp, Artificial sequence
  Synthetic oligonucleotide
ugugaaagugauccgcgactt

SEQ ID NO: 17 - DNA, 21 bp, Artificial sequence
  Synthetic oligonucleotide
gucgcggaucacuuucacatt

SEQ ID NO: 18 - DNA, 21 bp, Artificial sequence
  Primer
ctcctccggctggtgcatttt

SEQ ID NO: 19 - DNA, 21 bp, Artificial sequence
  Scrambled siRNA oligonucleotide
ugagaaugugaugcgcguctt

SEQ ID NO: 20 - DNA, 21 bp, Artificial sequence
  Scrambled siRNA oligonucleotide
gacgcgcaucacauucucatt